SEQUENCE LISTING (19 sequences)

SEQ ID NO: 1 - protein, 7 aa, Artificial Sequence
  Synthetic
LVFFAED

SEQ ID NO: 2 - protein, 13 aa, Artificial Sequence
  Synthetic
AMVTTACHEFFEH

SEQ ID NO: 3 - protein, 10 aa, Artificial Sequence
  Synthetic
KGVVKAEKSK

SEQ ID NO: 4 - protein, 6 aa, Artificial Sequence
  Synthetic
VVAAEE

SEQ ID NO: 5 - protein, 7 aa, Artificial Sequence
  Synthetic
EEAAVVK

SEQ ID NO: 6 - protein, 4 aa, Artificial Sequence
  Synthetic
RYDF

SEQ ID NO: 7 - protein, 5 aa, Artificial Sequence
  Synthetic
TPTQQ

SEQ ID NO: 8 - protein, 9 aa, Artificial Sequence
  Synthetic
GNGSGYVSR

SEQ ID NO: 9 - protein, 13 aa, Artificial Sequence
  Synthetic
AMVTTAAHEFFEH

SEQ ID NO: 10 - protein, 18 aa, Artificial Sequence
  Synthetic
VVAAEEGGKGVVKAEKSK

SEQ ID NO: 11 - protein, 15 aa, Artificial Sequence
  Synthetic
VVAAEEGGLVFFAED

SEQ ID NO: 12 - protein, 12 aa, Artificial Sequence
  Synthetic
VVAAEEGGRYDF

SEQ ID NO: 13 - protein, 14 aa, Artificial Sequence
  Synthetic
TPTQQGGEEAAVVK

SEQ ID NO: 14 - protein, 6 aa, Artificial Sequence
  Synthetic
TPTQQS

SEQ ID NO: 15 - protein, 7 aa, Artificial Sequence
  Synthetic
VVAAEEK

SEQ ID NO: 16 - protein, 17 aa, Artificial Sequence
  Synthetic
VVAAEEGGGNGSGYVSR

SEQ ID NO: 17 - protein, 21 aa, Artificial Sequence
  Synthetic
VVAAEEGGAMVTTAAHEFFEH

SEQ ID NO: 18 - protein, 10 aa, Artificial Sequence
  Synthetic
QTSVSPSKVI

SEQ ID NO: 19 - protein, 10 aa, Artificial Sequence
  Synthetic
ITDGEATDSG

(19) United States
(12) Patent Application Publication
(10) Pub. No.: US 2023/0143731 A1
(43) Pub. Date: May 11, 2023

(54) NANOMATERIALS FOR TARGETED TREATMENT OF PULMONARY TISSUE

(71) Applicant: The University of North Carolina at Chapel Hill, Chapel Hill, NC (US)

(72) Inventors: Melina R. Kibbe, Chapel Hill, NC (US); Kathleen Marulanda, Chapel Hill, NC (US); Alexandra Mercel, Chapel Hill, NC (US); Nick Tsihlis, Chapel Hill, NC (US); Thomas Gregory Wyatt, Chapel Hill, NC (US)

(21) Appl. No.: 17/759,767

(22) PCT Filed: Feb. 1, 2021
(86) PCT No.: PCT/US2021/016052

§ 371 Date: Jul. 29, 2022

Related U.S. Application Data
(60) Provisional application No. 62/968,919, filed on Jan. 31, 2020.

(51) Int. Cl. A61K 38/00; C07K 14/78

(52) U.S. Cl. CPC A61K 38/00; C07K 14/78

(57) ABSTRACT

Provided herein are compositions and methods for targeted drug delivery to treat pulmonary injury. In particular, provided herein are nanoscale delivery vehicles for: drugs that treat pulmonary injury. Also provided here in are methods of generating the nanoscale delivery vehicles and compositions thereof.

### CROSS REFERENCE TO RELATED APPLICATIONS

This application claims priority from and the benefit of U.S. 62/968,919 filed Jan. 31, 2020, which is incorporated herein by reference.

### REFERENCE TO SEQUENCE LISTING

This application includes an electronic sequence listing in a file named 553420SEQLST.txt, created on Jan. 31, 2021 and containing 4,139 bytes, which is hereby incorporated by reference in its entirety for all purposes.

### BACKGROUND

Pulmonary hypertension is a progressive and severe condition associated with various clinical entities. The World Health Organization classifies pulmonary hypertension into five distinct groups based on the underlying mechanism and associated clinical conditions. A predicative indicator of mortality, the development of pulmonary hypertension results in an early and significant decline in survival in patients with chronic interstitial lung disease.⁵⁰ Poor prognosis is largely attributed to the development of obliterative, pathophysiological alterations secondary to pulmonary vascular remodeling. Chronic luminal obstruction leads to increased pulmonary vascular resistance, resulting in right heart failure and, subsequently, death.^{39,40,51} Despite remarkable therapeutic advancements over the last thirty years, mortality rates as high as 40% remain unacceptably high.³⁹

Current therapies largely target the three major regulatory pathways involved in vascular tone: nitric oxide, endothelin, and prostacyclin.³⁹ However, these options are plagued by considerable limitations including: short half-lives (minutes), formulary limitations, low bioavailability in diseased tissue, instability in acidic environments, and non-specific distribution. Consequently, patients suffer from systemic adverse effects such as hypotension, flushing, headaches, gastrointestinal symptoms, peripheral edema, anemia, hepatotoxicity, retinal vascular disease, and myocardial infarction.⁵² Furthermore, no superiority has been demonstrated between the different therapeutics or even within the same pharmacological class. The only exception is the use of intravenous (IV) prostacyclin, but its efficacy must be heavily balanced against its systemic adverse effects.³⁹ As such, despite a significant evolution in the management of pulmonary hypertension, prognosis remains poor and quality of life continues to suffer. Thus, there is a great need for new technology to adequately target and reverse pulmonary vascular remodeling alterations, and improve patient outcomes.

Similarly, smoke inhalation injury increases mortality of burn victims by over 20% and all current therapy remains supportive, including mechanical ventilation, antibiotics, fluid resuscitation, inhaled medications, and overall routine intensive care of patients. None of these specifically addresses the pathophysiology or underlying lung injury that is responsible for poor disease-related prognosis. Resultant pneumonia, acute lung injury, and acute respiratory distress syndrome are only some of the many devastating complications of this injury which can lead to multi-system organ failure and even death.^{[59]} There is no current established targeted therapy that can be administered to patients after smoke inhalation to directly treat the injured lung.

There remains a need to develop a targeted drug delivery approach provides maximal therapeutic effect to the pulmonary vasculature or injured lung tissue while minimizing off-target effects which thereby reduces systemic adverse effects.

### BRIEF SUMMARY

Compositions and methods are provided for targeting and treating a pulmonary injury or condition. In one aspect, provided herein are peptide amphiphiles comprising: (a) a hydrophobic non-peptidic segment; (b) a β-sheet-forming peptide segment; (c) a charged peptide segment; (d) a targeting moiety, wherein the targeting moiety localizes to pulmonary tissue; wherein the hydrophobic non-peptidic segment is covalently attached to the N-terminus or C-terminus of the β-sheet-forming peptide segment; wherein the β-sheet-forming peptide segment is covalently attached to the targeting moiety; and wherein the charged peptide segment is covalently attached to the targeting moiety. Also provided, are peptide amphiphiles further comprising a therapeutic agent. In embodiments, the pulmonary injury or condition is pulmonary hypertension or pulmonary injury due to smoke inhalation, cystic fibrosis, or chronic obstructive pulmonary disease. In embodiments, the targeting moiety comprises a peptide capable of localizing to an epitope of receptor for advanced glycation end-products (RAGE) or angiotensin-converting enzyme (ACE).

In another aspect, provided herein are self-assembled nanomaterials comprising a plurality of peptide amphiphiles, wherein said peptide amphiphiles comprise: (a) a hydrophobic non-peptidic segment; (b) a β-sheet-forming peptide segment; (c) a charged peptide segment; and (d) a targeting moiety, wherein the targeting moiety localizes to receptor for advanced glycation end products (RAGE) or angiotensin-converting enzyme (ACE); wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety. In embodiments, the self-assembled nanomaterial is a nanofiber. Also provided, are self-assembled nanomaterials further comprising a therapeutic agent.

In another aspect, provided herein are methods of treating a pulmonary injury or condition in a subject comprising, administering to the subject a composition comprising: one or more peptide amphiphile(s), wherein the peptide amphiphile comprises: (a) a hydrophobic non-peptidic segment; (b) a β-sheet-forming peptide segment; (c) a charged peptide segment; (d) a targeting moiety, wherein the targeting moiety localizes to receptor for advanced glycation end products (RAGE) or angiotensin-converting enzyme (ACE); and (e) a therapeutic agent; wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

In another aspect, provided herein are methods of treating a pulmonary injury or condition in a subject comprising, administering to the subject a composition comprising a self-assembled nanomaterial comprising: a plurality of peptide amphiphiles, wherein said peptide amphiphiles comprise: (a) a hydrophobic non-peptidic segment; (b) a β-sheet-forming peptide segment; (c) a charged peptide segment; (d) a targeting moiety, wherein the targeting moiety localizes to receptor for advanced glycation end products (RAGE) or angiotensin-converting enzyme (ACE); and (e) a therapeutic agent; wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

In another aspect, provided herein are methods of delivering a therapeutic agent to pulmonary tissue in a subject comprising, administering to the subject a composition comprising a self-assembled nanomaterial comprising: a plurality of peptide amphiphiles, wherein said peptide amphiphiles comprise: (a) a hydrophobic non-peptidic segment; (b) a β-sheet-forming peptide segment; (c) a charged peptide segment; (d) a targeting moiety, wherein the targeting moiety localizes to receptor for advanced glycation end products (RAGE) or angiotensin-converting enzyme (ACE); and (e) a therapeutic agent; wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

In another aspect, provided herein are methods of methods of making a peptide amphiphile (PA) based nanomaterial which targets receptor for advanced glycation end products (RAGE) or angiotensin-converting enzyme (ACE) comprising: synthesizing targeting PA molecules via solid phase peptide synthesis comprising contacting a RAGE-targeting peptide with a diluent PA backbone; purifying the PA molecules; dissolving targeting PA molecules and with a diluent PA in a molar ratio in a solvent; removing the solvent; and forming the nanomaterial via self-assembly by resuspending the mixture of PA molecules in liquid at physiological pH.

### BRIEF DESCRIPTION OF THE SEVERAL VIEWS OF THE DRAWING(S)

Having thus described the invention in general terms, reference will now be made to the accompanying drawings, which are not necessarily drawn to scale.

FIG. 1 illustrates PA design and transmission electron microscopy (TEM) images of PA co-assemblies. (A) Important PA regions. (B) Schematic of typical PA nanomaterial co-assembly. TEM imaging of (C) ACE-targeting PA and RAGE-targeting PA nanofibers.

FIG. 2 illustrates PA localization to pulmonary tissue after inhalation injury. (A) Fluorescence images (20λ objective) of uninjured and injured lungs. Rats were injected with 7.5 mg/mL non-targeted nanofiber (diluent PA), RAGE-targeted nanofiber (LVFF-PA), or ACE-targeted nanofiber (RYDF-PA, SEQ ID NO: 6). Red fluorescence (see arrows) indicates PA localization, blue (shown as light gray) is nuclei (stained with DAPI), and green (show as dark gray) is autofluorescence of the lung tissue. (B) Quantification of fluorescence shows 10-fold greater localization with ACE-targeted PA vs. RAGE-targeted PA (***p<0.001).

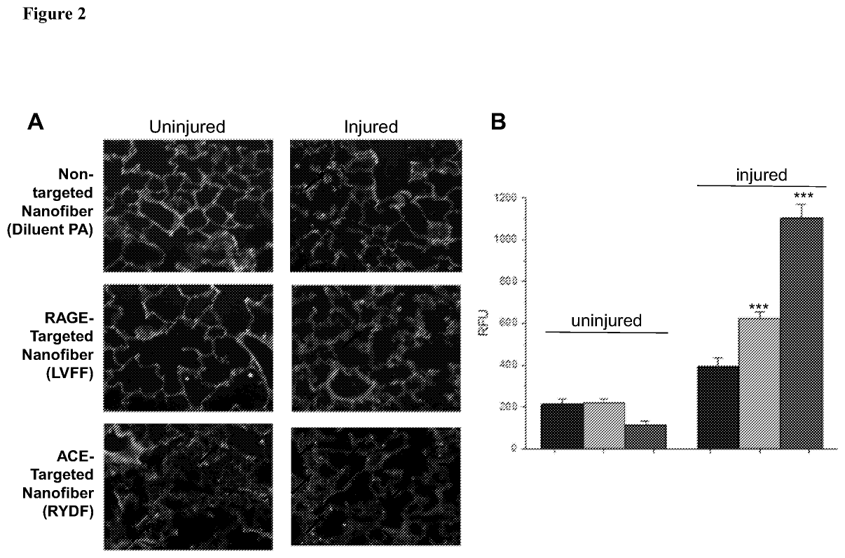

FIG. 3 illustrates PA localization to pulmonary tissue after hypoxia-induced pulmonary hypertension. Light sheet fluorescence microscopy (LSFM) images of a mouse lung following injection with 50 mole percent (1:1 ratio) of RAGE-targeted PA (“LVFF”) at 20 mg/mL dose. Images taken at 0.63× magnification. Red fluorescence (shown as dark gray) indicates PA localization and green (show as light gray) is autofluorescence of the lung tissue.

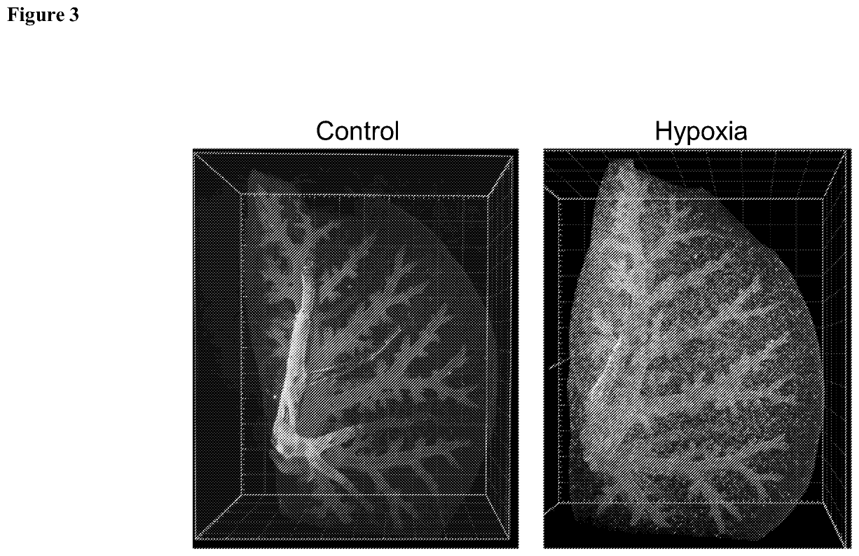

FIG. 4 is a schematic of rat model of smoke inhalation injury with peptide amphiphile nanofiber injection.

FIG. 5 illustrates chemical structure of synthesized peptide amphiphiles. Five total sequences were tested including 2 targeted to ACE: (A) RYDF (SEQ ID NO: 6) and (B) TPTQQ (SEQ ID NO: 7) and 3 targeted to RAGE: (C) “AMVTT” (SEQ ID NO: 2), (D) “KGVV” (SEQ ID NO: 3), and (E) “LVFF” (SEQ ID NO: 1). Residues in purple indicate the targeting region of each peptide amphiphile.

FIG. 6 illustrates confirmation of nanofiber formation by TEM. Each nanofiber was made with molar ratios from 25 to 100%. All samples were dissolved to a concentration of 0.5 mg/mL in HBSS, mounted onto carbon foils, and stained with 2% uranyl acetate. Scale bar=500 nm.

FIG. 7 illustrates characterization of non-targeted backbone C₁₆-VVAAEE (SEQ ID NO: 4) nanofiber. (A) Molecular graphics representation of individual peptide amphiphile molecules, with C₁₆-VVAAEE (SEQ ID NO: 4) in blue and fluorescent TAMRA label in red. (B) Molecular graphics model of 3D structure of co-assembled non-targeted nanofiber with 95 mole % non-targeted C₁₆-VVAAEE (SEQ ID NO: 4) backbone and 5 mole % TAMRA-labeled C₁₆-VVAAEE (SEQ ID NO: 4) backbone. (C) Confirmation of nanofiber formation on conventional TEM and by (D) circular dichroism spectroscopy. Scale bar=500 nm.

FIG. 8 illustrates ACE and RAGE protein levels were elevated after smoke inhalation injury. Lung sections (10 μm) from sham animals were compared to lungs from smoke-injured animals to evaluate levels of (A) ACE, (B) RAGE, (C) ACE, and (D) RAGE. Quantification of fluorescence intensity of (E) ACE and (F) RAGE. N=3/group. Each dot is representative of 16 images per individual animal. Scale bar=50 μm. **p<0.001 vs. sham. Blue=DAPI nuclear stain, Green=lung autofluorescence, Red=target protein.

FIG. 9 illustrates lung-targeted nanofibers localized to injured pulmonary tissue after smoke inhalation injury. (A) Localization of 5 targeted peptide amphiphile nanofibers in sham lungs versus smoke-injured lungs. N=3-6/group, each dot represents an average of 16 images per individual animal. Green=lung autofluorescence, Red=TAMRA-labeled nanofiber. Scale bar=50 μm. (B) Quantification of fluorescence intensity of each nanofiber compared to non-targeted control nanofiber. *p<0.05 for “KGVV” (SEQ ID NO: 3) targeted nanofibers in injured lung vs. sham. ***p<0.001 for RYDF (SEQ ID NO: 6) and “LVFF” (SEQ ID NO: 1) targeted nanofibers in injured lung vs. sham and RYDF (SEQ ID NO: 6) target nanofibers vs. “LVFF” (SEQ ID NO: 1) targeted nanofibers in injured lung. ###p<0.001 for RYDF (SEQ ID NO: 6) and “LVFF” (SEQ ID NO: 1) targeted nanofibers in injured lung vs. non-targeted control nanofibers in injured lung.

FIG. 10 illustrates RYDF (SEQ ID NO: 6) nanofiber optimization by epitope ratio and dosage allowed for maximal lung localization after smoke inhalation injury. (A) Immunofluorescence microscopy evaluating localization of non-targeted backbone nanofiber and RYDF (SEQ ID NO: 6)-targeted nanofiber at three different mole percentages: 25%, 50%, and 75%. Green=lung autofluorescence, Red=TAMRA-labeled nanofiber. Scale bar=50 μm. (B) Quantification of fluorescence intensity at each epitope ratio. N=3-6/group, each dot represents an average of 16 images per individual animal. ***p<0.001 for 75% vs. 50% RYDF (SEQ ID NO: 6) and 75% vs. non-targeted.

FIG. 11 illustrates characterization of 75 mole % RYDF (SEQ ID NO: 6) peptide amphiphile nanofiber. (A) Molecular graphics representation of individual peptide amphiphile molecules, with (I) non-targeted C₁₆-VVAAEE (SEQ ID NO: 4) backbone in blue, (II) fluorescent TAMRA label in red, and (III) ACE-targeting sequence RYDF (SEQ ID NO: 6) in purple. (B) Fiber formation in serum confirmed using cryogenic TEM. Scale bar=500 nm. X-ray scattering analysis using (C) small-angle X-ray scattering (SAXS) and (D) wide-angle X-ray scattering (WAXS) for 75 mole % RYDF (SEQ ID NO: 6) nanofiber. Open circles representing the scattering intensity versus wave vector data were fit to a polydisperse core-shell cylinder model (solid line). Confirmation of β-sheet structure using circular dichroism spectroscopy analysis of (E) 75 mole % RYDF (SEQ ID NO: 6) nanofiber and (F) 75 mole % RYDF (SEQ ID NO: 6) nanofiber (red line) vs. non-targeted backbone (black line).

FIG. 12 illustrates RYDF (SEQ ID NO: 6) nanofiber exhibited optimal dosage and was detectable in lungs up to 24 hours after injury. (A) Dosage study of 75 mole % RYDF (SEQ ID NO: 6) nanofiber evaluated by immunofluorescence with 5 mg and 7.5 mg tested in both sham and smoke-injured animals. N=3-7/group. (B) Quantification of fluorescence intensity at different dosages of nanofiber. Each dot represents an average of 16 images per individual animal. ***p<0.001 injury vs. sham. (C) Localization duration of 5 mg 75 mole % RYDF (SEQ ID NO: 6) nanofiber after 1 hour, 4 hours, and 24 hours compared to non-targeted control after 1 hour of circulation time. (D) Quantification of fluorescence intensity. N=3-4/group. Green=lung autofluorescence, Red=TAMRA-labeled nanofiber. Scale bar=50 μm.

FIG. 13 illustrates RYDF (SEQ ID NO: 6) nanofiber predominantly localizes to the lungs. (A) Immunofluorescence images of organ biodistribution of 5 mg 75 mole % RYDF (SEQ ID NO: 6) nanofiber. (B) Quantification of fluorescence intensity of each organ at varying timepoints. N=3-4/group. Green=lung autofluorescence, Red=TAMRA-labeled nanofiber. Scale bar=50 μm. Each dot is representative of average arbitrary units (AU) per animal, with 16 images per animal. ***p<0.001 in liver tissue of non-targeted and targeted nanofibers at 1 hour time point vs. other organs at the same time point.

FIG. 14 illustrates structure and characterization of ACE- and RAGE-targeted PA nanofibers. Chemical structures of (A) ACE-targeted PAs: “GNG” PA (SEQ ID NO: 16), RYDF PA (SEQ ID NO: 12), and “TPTQ” PA (SEQ ID NO: 13), and (B) RAGE-targeted PAs: “AMV” PA (SEQ ID NO: 9), “KGVV” PA (SEQ ID NO: 10), and “LVFF” PA (SEQ ID NO: 11). (C) Representative TEM images of all targeted nanofibers reconstituted in 1 mg/mL in HBSS at varying co-assembly molar ratios. Both targeting epitope and co-assembly molar ratios influenced fiber formation. Scale bar: 500 nm.

FIG. 15 illustrates hypoxia-induced pulmonary hypertension in CBL57/6 mice. (A) Hematoxylin and eosin-stained lungs demonstrating increased vessel wall muscularization (black arrows) in hypoxic vs. normoxic mice. Scale bar: 50 (B) The number of non-muscularized, partially and fully muscularized small (25-75 μm) pulmonary vessels was quantified in normoxic vs. hypoxic mice (n=9-10). *P<0.05, **P<0.01, ***P<0.001; Kruskal-Wallis test. (C) Immunofluorescence staining of smooth muscle cell (SMC) α-actin (red) in pulmonary vessels (white arrows). Green is tissue autofluorescence. Blue is DAPI stain (nuclei). Scale bar: 50 (D) Increased SMC α-actin levels indicate hypermuscularization of pulmonary vasculature in hypoxic mice (n=6). ***P<0.001; Mann Whitney test. Echocardiographic findings in normoxic vs. hypoxic mice comparing (E) pulmonary artery acceleration time (PAT), (F) pulmonary artery velocity time index (VTI), and (G) pulmonary vascular resistance (PVR) demonstrate elevated arterial pressures in hypoxic mice (n=11). *P<0.05; paired Student's t-test. (H) Right ventricular systolic pressure (RVSP) waveform tracing demonstrates elevated pressures in a mouse with hypoxia-induced pulmonary hypertension. (I) Quantification of RVSP in normoxic vs. hypoxic mice (n=6-11). **P<0.01; 2-sample Student's t-test. In B, D-G, and I, data are expressed as mean±standard error of the mean (SEM) and each dot represents an individual animal.

FIG. 16 illustrates increased ACE and RAGE levels in CBL57/6 mice with chronic hypoxia-induced pulmonary hypertension. (A) Representative images of immunofluorescence staining of ACE (red) and RAGE (red) in normoxic vs. hypoxic lungs. Blue=DAPI nuclear staining. Green=lung autofluorescence. Scale bar: 50 μm. Quantification of lung (B) ACE fluorescence intensity (n=6) and (C) RAGE fluorescence intensity (n=7) for normoxic vs. hypoxic mice. Data are arbitrary units (a.u.) expressed as mean±SEM. *P<0.01, **P<0.001; Mann Whitney test. Each dot represents an individual result; 16 images were analyzed per animal.

FIG. 17 illustrates RAGE-targeted “LVFF” (SEQ ID NO: 1) nanofiber localizes to lung with hypoxia-induced pulmonary hypertension. Normoxic and hypoxic mice were injected with ACE- and RAGE-targeted nanofibers. (A) Representative image of 3D LSFM demonstrating fluorescence localization (red) of nanofibers in normoxic vs. hypoxic mouse lungs. Lung autofluorescence is represented in green. Scale bar: 1500 μm. (B) Comparison of normoxic vs. hypoxic mice injected with non-targeted VVAAEE (SEQ ID NO: 4) nanofiber (n=6-7), RYDF (SEQ ID NO: 6) nanofiber (n=5-7), “TPTQ” nanofiber (n=5-6) (SEQ ID NO: 7), “AMV” (SEQ ID NO: 9) nanofiber (n=5-7), “KGVV” (SEQ ID NO: 3) nanofiber (n=4-5) and “LVFF” (SEQ ID NO: 1) nanofiber (n=5-6). *P<0.05. (C) Distribution of “LVFF” (SEQ ID NO: 1) nanofiber throughout the lung in normoxic and hypoxic mouse lungs (n=5-6). (D) Male and female mice injected with “LVFF” (SEQ ID NO: 1) nanofiber had similar fluorescence levels in normoxic vs. hypoxic conditions (n=7 males, 4 females). Each dot represents an individual result. In B-D, data were analyzed by Kruskal-Wallis test with Bonferroni correction. Mean±SEM.

FIG. 18 illustrates “LVFF” (SEQ ID NO: 1) nanofibers colocalize to RAGE in hypoxic lung. Immunofluorescence staining of RAGE (purple) in hypoxic lungs from 4 mice injected with the “LVFF” (SEQ ID NO: 1) nanofiber (red). Green is tissue autofluorescence. Blue is DAPI stain (nuclei). Scale bar: 100 μm.

FIG. 19 illustrates inverse relationship between mole % of targeted epitope incorporated in “LVFF” (SEQ ID NO: 1) nanofiber co-assembly and lung localization. (A) Representative images of 3D LSFM demonstrating nanofiber lung localization (red) after injection of non-targeted VVAAEE (SEQ ID NO: 4) nanofibers vs. 25 mole %, 50 mole %, and 75 mole % “LVFF” (SEQ ID NO: 1) nanofiber co-assembly molar ratios in normoxic vs. hypoxic mice. Green is tissue autofluorescence. Scale bar: 1000 μm. (B) Quantification of nanofiber fluorescence in normoxic vs. hypoxic mice injected with non-targeted VVAAEE (SEQ ID NO: 4) nanofibers (n=6-7), 25 mole % “LVFF” (SEQ ID NO: 1) nanofibers (n=9), 50 mole % “LVFF” (SEQ ID NO: 1) nanofibers (n=5-6), and 75 mole % “LVFF” (SEQ ID NO: 1) nanofibers (n=6-9). Amongst hypoxic mice, 25 mole % “LVFF” (SEQ ID NO: 1) nanofibers had greater lung localization. *P<0.05, **P<0.01 compared to 25 mole % “LVFF” (SEQ ID NO: 1) nanofibers in hypoxia. All “LVFF” (SEQ ID NO: 1) co-assembly molar ratios had significantly more fluorescence in the hypoxic lung. #P<0.01, ##P<0.001 compared to respective normoxic group. (C) Quantification of 25 mole % “LVFF” (SEQ ID NO: 1) nanofiber localization between upper, middle, and lower regions of the lung in normoxic vs. hypoxic mice (n=9). Results in B and C were analyzed by Kruskal Wallis test with Bonferroni correction. (D) The number of individual 25 mole % “LVFF” (SEQ ID NO: 1) nanofiber fluorescence objects detected per lung volume in normoxic vs. hypoxic mice (n=9) demonstrating that nanofiber accumulation is evenly distributed rather than due to large clumps of nanofibers clustered together. ##P<0.001; Mann Whitney test. (E) Graph demonstrating relationship between 25 mole % “LVFF” (SEQ ID NO: 1) nanofiber fluorescence intensity and nanofiber fluorescence volume in a hypoxic mouse (a.u.=arbitrary units). Scale bar: 70 μm. (F) Male and female mice injected with 25 mole % “LVFF” (SEQ ID NO: 1) nanofiber had similar fluorescence levels in normoxic vs. hypoxic conditions (n=9 males and females, respectively). Data analyzed with Kruskal Wallis test. In B-D and F, data are expressed as mean±SEM and each dot represents an individual result; 4 images per animal were analyzed.

FIG. 20 illustrates dosing and targeting duration of 25 mole % “LVFF” (SEQ ID NO: 1) nanofiber. (A) Representative images of 3D LSFM demonstrating localization of 25 mole % “LVFF” (SEQ ID NO: 1) nanofiber (red) in hypoxic lungs at 5 mg/kg, 10 mg/kg, and 20 mg/kg. Green is tissue autofluorescence. Scale bar: 1500 μm. (B) Quantification of 25 mole % “LVFF” (SEQ ID NO: 1) nanofiber fluorescence in hypoxic mice at 5 mg/kg (n=7), 10 mg/kg (n=8), and 20 mg/kg (n=9). (C) Timeline of workflow for targeted nanofiber injection followed by return to normal activity until time of sacrifice at 30 min, 4 hrs, and 24 hrs, respectively. At each time interval, lungs were imaged with (D) LSFM to evaluate localization of 25 mole % “LVFF” (SEQ ID NO: 1) nanofiber (20 mg/kg, red). Green is tissue autofluorescence. Scale bar: 1500 μm. (E) Quantification of fluorescence in hypoxic mice at 30 min (n=9), 4 hrs (n=12), and 24 hrs (n=8) after injection with 25 mole % “LVFF” (SEQ ID NO: 1) nanofiber. In B and E, data expressed as mean±SEM. *P<0.01; Kruskal Wallis test with Bonferroni correction. Each dot represents an individual result; 4 images were analyzed per animal.

FIG. 21 illustrates off-target organ biodistribution of 25 mole % “LVFF” (SEQ ID NO: 1) nanofiber. (A) Representative images of 3D LSFM demonstrating 25 mole % “LVFF” (SEQ ID NO: 1) nanofiber localization (red) to liver, kidney, and heart at 30 min, 4 hrs, and 24 hrs after injection. Green is tissue autofluorescence. Scale bar: 1000 μm. (B) Quantification of off-target localization at 30 min (n=8-9), 4 hrs (n=12), and 24 hrs (n=7-8) in hypoxic mice. *P<0.05 for 4 hr liver vs. all other treatment groups; Kruskal Wallis test with Bonferroni correction. Each dot represents an individual result; 4 images were analyzed per animal. (C) Dose-response curve of fluorescence intensity (579 nm emission) for standardized amounts of 25 mole % “LVFF” (SEQ ID NO: 1) nanofiber. Red squares represent individual data points and red solid line represents the line of best fit. (D) Amount of nanofiber fluorescence excreted in the urine at 30 min (n=4), 4 hrs (n=8), and 24 hrs (n=7) post nanofiber injection in hypoxic mice vs. non-injected hypoxic controls (n=6). *P<0.001; one-way ANOVA with Tukey's test. Each dot represents an individual animal. (E) Representative cross-sectional LSFM image of kidney at 30 min vs. 4 hrs after injection with 25 mole % “LVFF” (SEQ ID NO: 1) nanofiber in hypoxic mice. Nanofiber accumulation migrates from the collecting duct (white arrow) to the renal parenchyma as time progresses with minimal remaining at 24 hrs (shown in panel A). Scale bar: 1000 μm. (F) Representative urine sample of hypoxic mice treated with 25 mole % “LVFF” (SEQ ID NO: 1) nanofiber at 30 min vs. 4 hrs after injection. Gross fluorescence is lost by 4 hrs. (G) Cumulative excretion of nanofiber fluorescence over time (30 min, n=4; 4 hrs, n=8; 24 hrs, n=7) compared to non-injected hypoxic controls (n=6). Excretion was calculated as a percentage of the total fluorescence provided by injection of intravenous 25 mole % “LVFF” (SEQ ID NO: 1) nanofiber. In B, D and E, data expressed as mean±SEM.

FIG. 22 illustrates high-performance liquid chromatography (HPLC) verified ≥95% purity and mass spectrometry analysis shows expected mass for ACE-targeted PAs: “GNG” (SEQ ID NO: 8) PA, RYDF (SEQ ID NO: 6) PA, and “TPTQ” (SEQ ID NO: 7) PA.

FIG. 23 illustrates HPLC verified ≥95% purity and mass spectrometry analysis shows expected mass for RAGE-targeted PAs: “AMV” (SEQ ID NO: 17) PA, “KGVV PA” (SEQ ID NO: 3), and “LVFF” (SEQ ID NO: 1) PA.

FIG. 24 illustrates HPLC verified ≥95% purity and mass spectrometry analysis shows expected mass for non-targeted PAs: EEAAVV-K-C₁₂ (SEQ ID NO: 5) PA and VVAAEE (SEQ ID NO: 4) PA.

FIG. 25 illustrates (A) Chemical structure of the non-targeted C₁₆-VVAAEE (SEQ ID NO: 4) backbone PA and (B) the non-targeted EEAAVV-K-C₁₂ (SEQ ID NO: 5) backbone PA. (C) Representative conventional TEM images of non-targeted C₁₆-VVAAEE (SEQ ID NO: 4) PA nanofiber and EEAAVV-K-C₁₂ (SEQ ID NO: 5) backbone PA nanofiber reconstituted in HBSS at 1 mg/mL concentration. Scale bar: 500 nm.

FIG. 26 illustrates characterization of 25% “LVFF” (SEQ ID NO: 1) PA nanofiber. (A) 3D molecular graphic of 25% “LVFF” (SEQ ID NO: 1) nanofiber formed with co-assembly of the following 3 PAs: (i.) non-targeted VVAAEE (SEQ ID NO: 4) backbone PA (70%), (ii.) fluorescently labeled non-targeted VVAAEE (SEQ ID NO: 4) backbone PA (5 mole %), and (iii.) “LVFF”- (SEQ ID NO: 1) targeted PA (25 mole %). (B) Representative image of 25 mole % “LVFF” (SEQ ID NO: 1) nanofiber on cryogenic TEM demonstrating nanofiber stability in serum-containing solution. Scale bar: 250 nm. Characterization of 25 mole % “LVFF” (SEQ ID NO: 1) nanofiber structure on (C) SAXS analysis. Open circles represent scattering intensity vs. wave vector. The data were fit to a polydisperse core-shell cylinder model represented by the solid black line. (D) WAXS analysis of 25 mole % “LVFF” (SEQ ID NO: 1) nanofiber peak intensity (solid purple line) is consistent with β-sheet structure. (E) Circular dichroism spectroscopy analysis for secondary structure of 25 mole % “LVFF” (SEQ ID NO: 1) nanofiber at 37° C. demonstrates β-sheet character.

FIG. 27 illustrates characterization of non-targeted VVAAEE (SEQ ID NO: 4) PA nanofiber on (A) SAXS analysis. Open circles represent scattering intensity versus wave vector. The data were fit to a polydisperse core-shell cylinder model represented by the solid black line. (B) WAXS analysis demonstrates peak intensity of the VVAAEE (SEQ ID NO: 4) PA nanofiber (solid blue line) at q=1.34 A⁻¹, which is consistent with spacing in β-sheet structures, as would be expected for our nanofiber assemblies. The second WAXS peak at q=1.55 A⁻¹ likely reflects packing of fiber filaments. (C) Circular dichroism spectroscopy analysis of non-targeted VVAAEE (SEQ ID NO: 4) PA nanofiber at 37° C. demonstrates β-sheet secondary structure based on the single minima around 220 nm.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Overview

Pulmonary hypertension is a complex disorder associated with severe, and often fatal, consequences. Current FDA-approved therapies aim to reduce vasoconstriction by targeting the major signaling pathways that regulate vascular tone.³⁹ Unfortunately, these therapies fail to modify the pulmonary vascular remodeling responsible for increased pulmonary vascular resistance, leading to cardiopulmonary collapse and ultimately death.⁴⁰ Despite significant medical advancements, a curative therapy remains elusive and available options offer only a modest improvement in morbidity and mortality.⁴¹ Thus, there is a clear need to develop a novel therapy that effectively manages this devastating disease.

All current therapies for pulmonary hypertension offer only supportive care; there is no cure available. Present therapies have considerable limitations including short half-lives (minutes), formulary limitations, low bioavailability in diseased tissue, instability in acidic environments, and non-specific distribution resulting in many systemic adverse effects. Thus, novel therapeutics are needed for pulmonary hypertension and other pulmonary injuries or conditions such as pulmonary injury due to smoke inhalation.

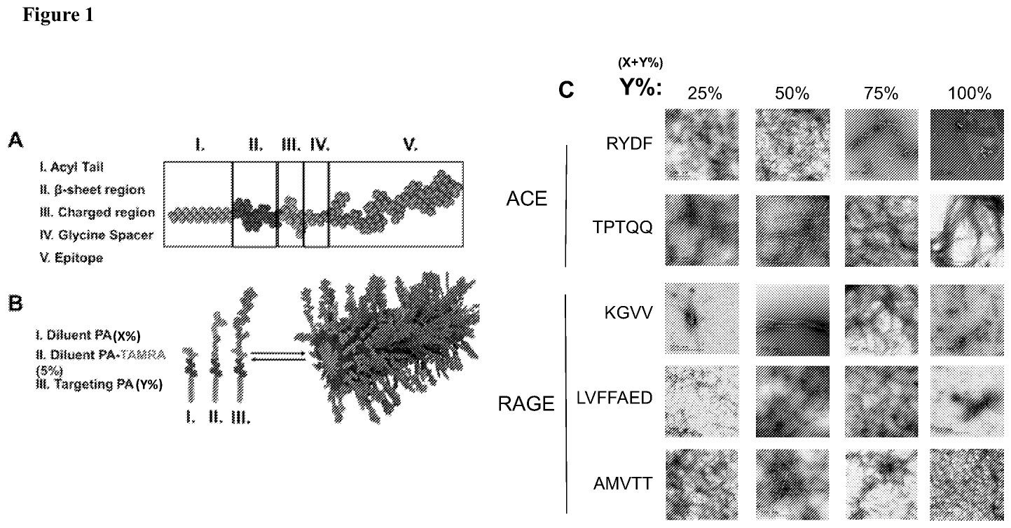

Using targeting moieties, nanoparticles can significantly increase drug efficacy by concentrating drug delivery to diseased tissues such as pulmonary tissue. Peptide amphiphile (PA)-based nanomaterials were selected as the targeting and drug delivery vehicle. PA molecules are comprised of an alkyl chain linked to an amino acid sequence which contains a β-sheet-forming region, a charged region, and epitope(s) that can target proteins of interest (FIG. 1). This platform was selected because PA molecules spontaneously form high aspect ratio supramolecular nanofibers to increase surface interactions of targeting epitopes while simultaneously serving as a drug delivery vehicle. Also, using intravascularly administered PA nanofibers targeted to prevent restenosis and to stop hemorrhage has been shown to be successful.

Provided herein, a nanoparticle drug delivery system that uses peptide amphiphiles, which spontaneously self-assemble into nanomaterials when placed in an aqueous environment. In embodiments, the aqueous environment is a liquid. In embodiments, the PAs are lyophilized. In embodiments, the PAs are reconstituted in a liquid prior to administration. In embodiments, the PAs are administered via intravascular, inhalational, or intratracheal delivery to form nanomaterials that can then target pulmonary tissue.

Therefore, in embodiments, the PA nanomaterials will target ACE or RAGE, which are significantly upregulated in pulmonary injury or conditions such as pulmonary hypertension or pulmonary injury due to smoke inhalation. In embodiments, the ACE- or RAGE-targeted PAs can also be co-assembled with other non-targeted PAs that are attached to therapeutics via covalent bonds or hydrophobic interactions. In embodiments, the attached therapeutic is a glutamine, a selectin or leukocyte adhesion molecule inhibitor, a CXCL-1 inhibitor, a perfluorohexane, an inducible nitric oxide synthase (iNOS) inhibitor, a neuronal NOS inhibitor, a peroxynitrite decomposition catalyst, a hydrogen sulfide (H₂S) via a hydrogen sulfide donor, a carvacrol, a nitric oxide, a phosphodiesterase type 5 (PDE5) inhibitor, a tyrosine kinase inhibitor, a peroxisome proliferator-activated receptor-gamma agonist, or a statin. In embodiments, the PAs can encapsulate hydrophobic drugs, which normally display poor oral bioavailability, and deliver them directly to the tissue of interest. Also provided herein, a novel biodegradable, biocompatible, intravenous, targeted nanoparticle drug delivery system that localizes to pulmonary tissue.

In embodiments, the subject matter described herein is directed to a nanomaterial for intravascular, inhalational, or intratracheal delivery to target pulmonary tissue. In embodiments, a nanoscale drug delivery system comprised of peptide amphiphile (PA) nanomaterials for intravascular, inhalational, or intratracheal delivery that targets the pulmonary tissue.

In embodiments, the subject matter described herein is directed to nanomaterials comprised of a synthetic peptide covalently attached to an aliphatic molecule. In embodiments, the peptide comprises a β-sheet-forming unit, a charged unit, and an optional epitope region. PA molecules spontaneously form nanomaterials in an aqueous environment. Using specific epitopes, the PA molecules can target proteins of interest. The nanofiber structure increases the surface area available to interact with target proteins, which can improve avidity of localization to targeted proteins.

In embodiments, the subject matter described herein is directed to PA molecules that further comprise covalently incorporated therapeutics directly onto PA monomers and/or incorporated hydrophobic drugs into the aliphatic core of the nanofiber structure. In embodiments, the therapeutic agent is covalently attached via a covalent bond or a hydrophobic interaction.

In embodiments, the subject matter described herein is directed to PA nanomaterials that target and deliver therapeutics. Multiple PA monomers can be co-assembled with both targeting PAs and PA monomers containing covalently attached therapeutics, and/or fluorescent tags.

In embodiments, the subject matter described herein is directed to PA molecules incorporating epitopes to target specific proteins overexpressed in a pulmonary injury or condition such as pulmonary hypertension or pulmonary injury due to smoke inhalation (ACE and RAGE), which is a feature exclusive to pulmonary injury.

In embodiments, the subject matter described herein is directed to PA nanomaterials comprising covalently incorporated nitric oxide-releasing molecules directly into the PA monomers, which were co-assembled with targeting monomers. Upon co-assembly these supramolecular nanomaterials target diseased tissue and activate localized release of nitric oxide.

### II. Definitions

As used herein, the term “nanofiber” refers to an elongated or threadlike filament (e.g., having a significantly greater length dimension than width or diameter) with a diameter of less than 100 nanometers.

As used herein, the term “nanosphere” refers to an approximately spherical (e.g., a globular shape having approximately (<25% difference, <10% difference, <5% difference) the same diameters in the x, y, and z dimensions) with a diameter of less than 500 nanometers (e.g., <200 nm, <100 nm, etc.).

As used herein, the term “supramolecular” (e.g., “supramolecular complex,” “supramolecular interactions,” “supramolecular fiber,” “supramolecular polymer,” etc.) refers to the non-covalent interactions between molecules (e.g., polymers, macromolecules, etc.) and the multicomponent assemblies, complexes, systems, and/or fibers that form as a result.

As used herein, the term “nanomaterial” refers to nanofibers, nanospheres, micelles, nanoribbons, and a variety of other structures that can be formed as a result of supramolecular interactions. In certain embodiments, the supramolecular interactions are between the peptide amphiphiles and other components in the nanomaterial.

As used herein, the term “physiological conditions” refers to the range of conditions of temperature, pH, and tonicity (or osmolality) normally encountered within tissues in the body of a living human.

As used herein, the terms “self-assemble,” “self-assembled,” and “self-assembly” refer to formation or product of a discrete, non-random, aggregate structure from component parts; said assembly occurring spontaneously through random movements of the components (e.g., molecules) due only to the inherent chemical or structural properties and attractive forces of those components. A “self-assembled nanofiber” refers to a product comprised of a plurality of peptide amphiphiles. As used herein, a “plurality” refers to two or more peptide amphiphiles.

As used herein, the term “peptide amphiphile” refers to a molecule that, at a minimum, includes a non-peptide lipophilic (hydrophobic) segment, a structural peptide segment, and optionally a functional peptide segment. The peptide amphiphile may express a net charge at physiological pH, either a net positive or negative net charge, or may be zwitterionic (i.e., carrying both positive and negative charges). Certain peptide amphiphiles consist of or comprise four segments: (1) a hydrophobic, non-peptidic segment comprising an acyl group of six or more carbons, (2) a β-sheet-forming peptide segment; (3) a charged peptide segment, and (4) a targeting moiety (e.g., targeting peptide).

As used herein and in the appended claims, the term “lipophilic component” or “hydrophobic component” refers to the acyl moiety disposed on the N-terminus (or C-terminus, depending on the orientation) of the peptide amphiphile. This lipophilic segment may be herein and elsewhere referred to as the aliphatic, lipophilic, or hydrophobic segment. The hydrophobic component should be of a sufficient length to provide amphiphilic behavior and micelle (or nanosphere or nanofiber) formation in water or another polar solvent system.

Accordingly, in the context of the embodiments described herein, the hydrophobic component preferably comprises a single, linear acyl chain of the formula: Cₙ₋₁H₂ₙ₋₁C(O)— where n=6-22. A particularly preferred single, linear acyl chain is the lipophilic group, palmitic acid. However, other small lipophilic groups may be used in place of the acyl chain.

As used herein, the term “structural peptide” or “β-sheet-forming peptide” refers to the intermediate amino acid sequence of the peptide amphiphile molecule between the hydrophobic segment and the charged peptide segment of the peptide amphiphile. This “structural peptide” or “β-sheet-forming peptide” is generally composed of three to ten amino acid residues with non-polar, uncharged side chains, selected for their propensity to form a β-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), and Gly (G) (listed in order of their propensity to form β-sheets). However, non-naturally occurring amino acids of similar β-sheet forming propensity may also be used. Peptide segments capable of interacting to form β-sheets and/or with a propensity to form β-sheets are understood (See, e.g., Mayo et al. Protein Science (1996), 5:1301-1315; herein incorporated by reference in its entirety). In a preferred embodiment, the N-terminus of the structural peptide segment is covalently attached to the oxygen of the lipophilic segment and the C-terminus of the structural peptide segment is covalently attached to the N-terminus of the charged peptide segment.

As used herein, the term “charged peptide segment” refers to the intermediately disposed peptide sequence between the structural peptide segment or β-sheet-forming segment and the functional peptide. In some embodiments, the charged segment provides for solubility of the peptide amphiphile in an aqueous environment, and preferably at a delivery location within a cell, tissue, organ, or subject. The charged peptide segment contains two or more amino acid residues that have side chains that are ionized under physiological conditions, examples of which selected from the 20 naturally occurring amino acids include Lys (K), Arg (R), Glu (E), and/or Asp (D), along with other uncharged amino acid residues. Non-natural amino acid residues with ionizable side chains could be used, as will be evident to one ordinarily skilled in the art. This segment may be from about 2 to about 7 amino acids long, and may be comprised of about 3 or 4 different amino acids. The charged peptide segment may include those amino acids and combinations thereof which provide this solubility and permit self-assembly and is not limited to polar amino acids such as E or K and combinations of these for modifying the solubility of the peptide amphiphile.

One or more Gly (G) residues may be added to the “charged peptide segment,” intermediately disposed between the charged residues and the functional peptide segment (e.g., targeting peptide). While not wishing to be bound by theory, the inclusion of one or more Gly (G) residues appears to prevent salt-bridge formation between the Glu and the Lys amino acid side-chains by altering side-chain orientation of these residues relative to each other, improving solubility of the peptide in salt solutions of similar composition to extracellular fluid. In one embodiment, the charged peptide segments have the formula (E)ₓ(G)_{y}, wherein x is 2 to 6 and y is 1 to 6. In another embodiment, the charged peptide segment has 2 to 4 Glu (E) residues and 1 to 2 Gly (G) residues. In another aspect, the charged peptide segment has 2 Glu (E) residues and 1 Gly (G) residue. In yet another aspect of the invention, the charged peptide segment has 3 Glu (E) residues and 1 Gly (G) residue. In another embodiment, the charged peptide segment has 4 Glu (E) residues and 1 Gly (G) residue. The glycine residues may also act as a spacer to provide greater accessibility of the targeting peptide to the protein of interest by extending the targeting peptide past the surface of the nanomaterial.

As used herein, the term “targeting peptide” refers to amino acid sequences which mediate the localization (or retention) of sequences, molecules, or supramolecular complexes associated therewith to a particular location or locations (e.g., sub-cellular location (e.g., organelle), an organ (e.g., heart), tissue (e.g., cardiovascular tissue), or localized with a receptor or binding partner for the targeting peptide)). Peptide amphiphiles and structures (e.g., nanofibers) bearing targeting peptides have been reported to congregate in desired locations based on the identity and presence of the targeting peptide. A targeting peptide described in exemplary embodiments herein is the RAGE- or ACE-targeting peptide. Such targeting peptides have been shown to deliver targeted nanomaterials comprising such peptides to pulmonary tissue. In embodiments, the targeting peptide acts by binding to and/or localizing to RAGE or ACE. In embodiments, the targeted nanomaterial binds to and/or localizes to sites where RAGE or ACE is expressed. In embodiments, the sites are sites of pulmonary injury, condition, or disease.

Proteins are said to have an “N-terminus” and a “C-terminus.” The term “N-terminus” relates to the start of a protein or polypeptide, terminated by an amino acid with a free amine group (—NH₂). The term “C-terminus” relates to the end of an amino acid chain (protein or polypeptide), terminated by a free carboxyl group (—COOH).

“Sequence identity” or “identity” in the context of two polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” refers to the value determined by comparing two optimally aligned sequences (greatest number of perfectly matched residues) over a comparison window, wherein the portion of the polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values refer to the value obtained using GAP Version 10 using the following parameters: % identity and % similarity for an amino acid sequence using GAP Weight of 8 and Length Weight of 2, and the BLOSUM62 scoring matrix; or any equivalent program thereof “Equivalent program” includes any sequence comparison program that, for any two sequences in question, generates an alignment having identical amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid, or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., amino acid sequence) refers to a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence.

The term “fragment” when referring to a protein means a protein that is shorter or has fewer amino acids than the full-length protein. A fragment can be, for example, an N-terminal fragment (i.e., removal of a portion of the C-terminal end of the protein), a C-terminal fragment (i.e., removal of a portion of the N-terminal end of the protein), or an internal fragment. A fragment can also be, for example, a functional fragment or an immunogenic fragment.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

Compositions or methods “comprising” or “including” one or more recited elements may include other elements not specifically recited. For example, a composition that “comprises” or “includes” a protein may contain the protein alone or in combination with other ingredients.

Designation of a range of values includes all integers within or defining the range, and all subranges defined by integers within the range.

Unless otherwise apparent from the context, the term “about” encompasses values within a standard margin of error of measurement (e.g., SEM) of a stated value or variations ±0.5%, 1%, 5%, or 10% from a specified value.

The singular forms of the articles “a,” “an,” and “the” include plural references unless the context clearly dictates otherwise. For example, the term “an antigen” or “at least one antigen” can include a plurality of antigens, including mixtures thereof.

Statistically significant means p≤0.05.

### III. Compositions of Matter

A. Peptide Amphiphile (PA)-Based Nanomaterials

Peptide amphiphiles (Hartgerink et al. P Natl Acad Sci USA 99, 5133 (2002); Hartgerink et al. Science 294, 1684 (2001); herein incorporated by reference in their entireties) (PAs) are a class of self-assembling molecules that are composed of a hydrophobic segment conjugated to a sequence of amino acids. PAs can form long, high aspect ratio cylindrical filaments in water and have been studied for a range of applications in regenerative medicine (Mata et al., Biomaterials 31, 6004 (2010); Shah et al., P Natl Acad Sci USA 107, 3293 (2010); Huang et al. Biomaterials 31, 9202 (2010); Webber et al., P Natl Acad Sci USA 108, 13438 (2011); herein incorporated by reference in their entireties). PA bioactivity is derived from presentation of peptide sequences on the surface of self-assembled nanostructures that form in solution. The rheological properties of these materials can be tuned by concentration and peptide sequence (Pashuck et al. Journal of the American Chemical Society 132, 6041 (2010); herein incorporated by reference in its entirety).

Nanoparticle-mediated drug delivery systems have been used in many aspects of therapeutic research due to size, solubility, protection against degradation, and carrier capacity. Inhaled nanotherapeutics have been of specific interest to lung diseases in an effort to treat infection, disease, or cancer via aerosolized delivery targeted to lung tissue.^{[60,61]} Inhaled nanotherapy, however, has limitations. Aerosolized particles have poor pulmonary deposition after inhalation and pulmonary clearance of suspended particles inhibits access to lung epithelial cells and distal airways.^{[62,63]} These challenges increase support for the development of a systemically administered, lung-targeted drug delivery system.

Self-assembling peptide amphiphile (PA) nanofibers are of particular interest to the drug delivery field. Self-assembled PA nanofibers are biocompatible nanomaterials that can be modified to recognize specific biological markers to provide targeted drug delivery and reduce off-target toxicity. These fibers consist of a hydrophobic carbon chain that allows for self-assembly into a cylindrical micelle in aqueous solution and a hydrophilic head region that contains a specific epitope to achieve targeted delivery.^{[64]} Additionally, PA nanofibers are considerably smaller than other nanoparticles tested for pulmonary hypertension, with diameters ranging from 6-8 nm.^{[114,116]} Importantly, a therapeutic payload can be either loaded into the hydrophobic core or covalently attached to the hydrophilic region.

Examples of peptide amphiphile (PA)-based nanomaterials discussed herein can be found in U.S. Pat. No. 9,517,275; herein incorporated by reference in its entirety. In some embodiments, provided. herein are peptide amphiphiles comprising: (a) a hydrophobic non-peptidic segment; (b) a β-sheet-forming peptide segment; (c) a charged peptide segment; (d) a targeting moiety; and (e) a therapeutic agent. In some embodiments, the hydrophobic non-peptidic segment is covalently attached to the N-terminus of the β-sheet-forming peptide segment; wherein the C-terminus of the β-sheet-forming peptide segment is covalently attached to the N-terminus of the charged peptide segment; and wherein the C-terminus of the charged peptide segment is covalently attached to the N-terminus of the targeting moiety. In some embodiments, the hydrophobic non-peptidic segment is covalently attached to the C-terminus of the β-sheet-forming peptide segment; wherein the N-terminus of the β-sheet-forming peptide segment is covalently attached to the C-terminus of the charged peptide segment; and wherein the N-terminus of the charged peptide segment is covalently attached to the C-terminus of the targeting moiety. in some embodiments, the hydrophobic non-peptidic segment comprises an acyl chain. In some embodiments, the acyl chain comprises C₆-C₂₄ (e.g., C₆ . . . C₈ . . . C₁₀ . . . C₁₂ . . . C₁₄ . . . C₁₆ . . . C₁₈ . . . C₂₀ . . . C₂₂ . . . C₂₄). In some embodiments, the acyl chain comprises lauric acid. in some embodiments, the β-sheet-forming peptide segment comprises AAVV. in some embodiments, the charged peptide segment comprises a plurality of Lys (K), Arg (R), Glu (E), and/or Asp (D) residues. In some embodiments, the charged peptide segment comprises 2-7 amino acids in length with 50% or more amino acids selected from Lys (K), Arg (R), Glu (E), and/or Asp (D) residues, In some embodiments, the charged peptide segment comprises KK. in some embodiments, the targeting moiety comprises a targeting sequence for a protein of interest. In some embodiments, the target protein is RAGE or ACE. In some embodiments, the therapeutic agent is covalently linked to the peptide amphiphile. In some embodiments, the therapeutic agent is nitric oxide (NO). In some embodiments, the NO is covalently linked to the peptide amphiphile as a nitroso group. In some embodiments, the nitroso group is attached via nitrosylation of a cysteine residue. in some embodiments, the peptide amphiphile contains an S-nitrosylated cysteine residue.

In embodiments, the therapeutic agent is a therapeutic agent selected from Table 1.

TABLE 1
Examples of therapeutic agents.

| | | | MW |
|---|---|---|---|
| Therapeutic | Target | Molecule | (g/mol) |
| Glutamine | IKKβ (indirectly) | 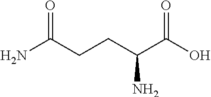 | 217.2 |
| QTSVSPSKVI (SEQ ID NO: 18) | LFA-1 at MIDAS site | 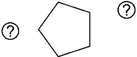 | 1044.38 |
| ITDGEATDSG (SEQ ID NO: 19) | ICAM-1 | 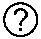 | 964.93 |
| N- acetylcysteine | ROS (antioxidant) | 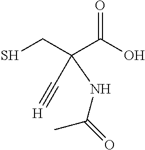 | 163.19 |
| Ascorbic acid | ROS (antioxidant) | 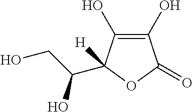 [SMILES: O=C1O[C@H]([C@@H](O)CO)C(O)=C1O] | 176.12 |

In some embodiments, provided herein are self-assembled nanomaterials formed of the peptide amphiphiles described above (or elsewhere herein). in some embodiments, the nanofiber has a diameter of less than 200 nm, <150 nm, <100 nm, <50 nm). In some embodiments, the nanofiber has a diameter of 10-200 nm (e.g., 20-180 nm, 50-200 nm, 30-150 nm, or other ranges less than 200 nm and greater than 10 nm). In some embodiments, the nanofiber has a length of at least 1 μm. In some embodiments, the nanofiber has a length of at least 500 nm to 50 μm (e.g., >500 nm, >1 μm, >2 μm, >5 μm, >10 μm, <50 μm, <40 μm, <30 μm, <20 μm, etc.).

In some embodiments, provided herein are supramolecular nanostructures (e.g., formed by self-assembly of a single molecule type) that target the site of vascular injury and deliver therapeutic (e.g., NO). An exemplary molecular building block for the supramolecular nanostructures is a peptide amphiphile (PA) containing a peptide segment conjugated to an aliphatic tail. This broad family of molecules is in the creation, assembly, and/or manufacture of bioactive nanostructures for regenerative medicine and drug delivery (Cui, et al. Biopolymers, Vol. 94 1-18 (2010); Matson & Stupp. Chem. Commun, Vol. 48 26 (2011); Webber, M. J., et al. Proceedings of the National Academy of Sciences, Vol. 108 13438-13443 (2011); Matson, et al. Soft Matter, Vol. 8 6689 (2012); Soukasene, S., et al. ACS Nano, Vol. 5 9113-9121 (2011); herein incorporated by reference in their entireties). PAs are made to self-assemble into nanostructures of various shapes, including spheres and fibers, by altering the peptide sequences (Muraoka et al. Angew. Chem. Int. Ed., Vol. 48 5946-5949 (2009); Cui et al. Nano Lett., Vol. 9 945-951 (2009); Paramonov et al. J. Am. Chem. Soc., Vol. 128 7291-7298 (2006); herein incorporated by reference in their entireties). This ability is attractive to vascular applications because a filamentous shape has been previously shown to extend circulation time and bind to the endothelium (Geng, Y., et al. Nature Nanotechnology, Vol. 2 249-255 (2007); Shuvaev, V. V., et al. ACS Nano, Vol. 5 6991-6999 (2011); herein incorporated by reference in their entireties). The peptide portion of a PA is also an ideal site to integrate various bioactive functions.

In some embodiments, the peptide amphiphile molecules and compositions of the embodiments described herein are synthesized using preparatory techniques well-known to those skilled in the art, preferably, by standard solid-phase peptide synthesis, with the addition of a fatty acid in place of a standard amino acid at the N-terminus of the peptide, in order to create the lipophilic segment. Synthesis typically starts from the C-terminus, to which amino acids are sequentially added using either a Rink amide resin (resulting in an —NH₂ group at the C-terminus of the peptide after cleavage from the resin), or a Wang resin (resulting in an —OH group at the C-terminus). Accordingly, embodiments described herein encompasses peptide amphiphiles having a C-terminal moiety that may be selected from the group consisting of —H, —OH, —COOH, —CONH₂, and —NH₂.

The lipophilic segment is typically incorporated at the N-terminus of the peptide after the last amino acid coupling and is composed of a fatty acid or other acid that is linked to the N-terminal amino acid through an acyl bond. Additionally, the lipophilic segment can be incorporated at the C-terminus via an acyl bond to a lysine side chain. In aqueous solutions, PA molecules self-assemble (e.g., into cylindrical micelles (a.k.a., nanofibers)) that bury the lipophilic segment in their core and display the functional peptide on the surface. The structural peptide undergoes intermolecular hydrogen bonding to form β-sheets that orient parallel to the long axis of the micelle.

In some embodiments, compositions described herein comprise PA building blocks that in turn comprise a hydrophobic segment and a peptide segment. In certain embodiments, a hydrophobic (e.g., hydrocarbon and/or alkyl tail) segment of sufficient length (e.g., >3 carbons, >5 carbons, >7 carbons, >9 carbons, etc.) is covalently coupled to peptide segment (e.g., an ionic peptide having a preference for β-strand conformations) to yield a peptide amphiphile molecule. In some embodiments, a plurality of such PAs will self-assemble in water (or aqueous solution) into a nanostructure (e.g., nanofiber). In various embodiments, the relative lengths of the peptide segment and hydrophobic segment result in differing PA molecular shape and nanostructural architecture. For example, a broader peptide segment and narrower hydrophobic segment results in a generally conical molecular shape that has an effect on the assembly of PAs (See, e.g., J. N. Israelachvili Intermolecular and surface forces; 2nd ed.; Academic: London San Diego, 1992; herein incorporated by reference in its entirety). Other molecular shapes have similar effects on assembly and nanostructural architecture. In various embodiments, hydrophobic segments pack in the center of the assembly with the peptide segments exposed to an aqueous or hydrophilic environment to form cylindrical nanostructures that resemble filaments. Such nanofilaments display the peptide regions on their exterior and have a hydrophobic core.

To induce self-assembly of an aqueous solution of peptide amphiphiles, the pH of the solution may be changed (raised or lowered) or multivalent ions, such as calcium, or charged polymers or other macromolecules may be added to the solution. Though not intending to be bound by theory, self-assembly is facilitated in the instant case by the neutralization or screening (reduction) of electrostatic repulsion between ionized side chains on the charged peptide segment.

In some embodiments, the hydrophobic segment is a non-peptide segment (e.g., alkyl group). In some embodiments, the hydrophobic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, fluorinated alkyl tails, aromatic segments, pi-conjugated segments, etc.

In some embodiments, peptide amphiphiles comprise a targeting moiety. In particular embodiments, a targeting moiety is the C-terminal most segment of the PA. In some embodiments, the targeting moiety is attached to the C-terminal end of the charged segment. In some embodiments, the targeting moiety is exposed on the surface of an assembled PA structure (e.g., nanofiber). A targeting moiety is typically a peptide (e.g., targeting peptide), but is not limited thereto. For example, in some embodiments, a targeting moiety is a small molecule (e.g., the target for a receptor, a ligand for a protein, etc.). Examples described in detail herein utilize a peptide sequence that localizes to RAGE or ACE. The presence of the RAGE- or ACE-targeting sequence directs the PA structures (e.g., nanofibers) to the pulmonary tissue, allowing them to localize at the site of interventions (e.g., to isolate the therapeutic action at the desired site). Further, targeting moieties may localize to (and thereby direct PA structures to) proteins or other targets that are localized in other regions of the body, or even subcellular locations. Targeting moieties may direct PA structures (and therefore the therapeutics attached thereto or encapsulated therein) to specific organs, tissues, cell types, subcellular locations (e.g., organelles), pathogens (e.g., viruses, bacteria, etc.), diseases (e.g., to cancerous cells), etc. Targeting peptides and other moieties for achieving such localization are understood. As additional targeting moieties are discovered, they too may find use in embodiments described herein.

Suitable peptide amphiphiles, PA segments, PA nanostructures, and associated reagents and methods are described, for example in U.S. Pat. Nos. 8,512,693; 8,450,271; 8,138,140; 8,124,583; 8,114,835; 8,114,834; 8,080,262; 8,063,014; 7,851,445; 7,838,491; 7,745,708; 7,683,025; 7,554,021; 7,544,661; 7,534,761; 7,491,690; 7,452,679; 7,390,526; 7,371,719; 6,890,654; herein incorporated by reference in their entireties.

In certain embodiments, peptide amphiphiles further comprise a therapeutic group. In some embodiments, a therapeutic (e.g., a drug that prevents proliferation and neointimal hyperplasia (e.g., NO)) is covalently or non-covalently attached to PA. For example, a therapeutic is attached to a PA such that it is exposed on the surface of the assembled PA structure (e.g., nanofiber). In some embodiments, a therapeutic is covalently linked to the peptide portion of the PA. In some embodiments, any suitable chemistry known to those in the art is used for the covalent attachment (e.g., modification of a cysteine in the PA (e.g., S-nitrosylation)). In other embodiments, a therapeutic is attached to PA such that it is released (e.g., in a burst, over time, upon exposure to particular conditions, etc.) from the PA and/or assembled PA structure (e.g., nanofiber). In some embodiments, a therapeutic is not attached to the individual PAs, but is incorporated into or encapsulated within a PA supramolecular structure. In embodiments, the therapeutic agent is attached to the PA by a covalent bond or incorporated into the nanomaterial core by hydrophobic or hydrophilic interaction. In such embodiments, the therapeutic is released from the structure at a desired rate and/or under desired conditions (e.g., physiological conditions, upon localization of the targeting moiety to a target, etc.).

Exemplary therapeutic groups include small molecules (e.g., NO), peptides, antibodies, nucleic acids (e.g., siRNA, antisense RNA, etc.), etc. Examples described in detail herein utilize nitric oxide as a therapeutic. In the examples, PAs were S-nitrosylated (e.g., SNO groups added to the PAs). Upon degradation of the SNO groups, NO is released from the assembled PA structure (e.g., nanofiber). Therapeutic delivery of NO is not limited to S-nitrosylation of PAs. Further, embodiments are not limited to delivery of NO. Any therapeutic that can be delivered and localized to a desired site of action (e.g., by a targeting moiety) finds use in embodiments described herein. For example, drugs that prevent proliferation and neointimal hyperplasia may be delivered to sites of arterial intervention to reduce and/or prevent restenosis in the cardiovascular system. Exemplary drugs for such use include, but are not limited to: nitric oxide, acetylsalicylic acid, rapamycin, paclitaxel, etc.

The characteristics (e.g., shape, rigidity, hydrophilicity, etc.) of a PA supramolecular structure depend upon the identity of the components of a peptide amphiphile (e.g., lipophilic segment, charged segment, structural segment, functional segment, etc.). For example, nanofibers, nanospheres, intermediate shapes, and other supramolcular structures are achieved by adjusting the identity of the PA component parts. In examples provided herein, the fiber shape of the nanoscale delivery vehicle proved particularly conducive to cardiovascular applications, and exhibited significant and measurable advantage over, for example nanosphere delivery vehicles. In other embodiments, for example, when a different site of action is desired, other vehicle characteristics may be desirable. In some embodiments, provided herein are nanoscale delivery vehicles with tunable shapes to best suit the intended therapeutic delivery location. For example, nanofibers may be preferred over nanospheres for a particular delivery site (e.g., site of vascular intervention). Likewise, in some embodiments, a particular length to diameter ratio (or range of ratios) is particularly advantageous for a delivery location.

In certain embodiments, PAs and the nanofibers assembled therefrom comprise a targeting moiety configured to deliver the PA and/or nanomaterial to a desired location within a cell, tissue, organ, body system, or subject (e.g., human, non-human primate, rodent, etc.). In some embodiments, a PA and/or nanomaterial is also associated with (e.g., covalently or non-covalently) a therapeutic agent configured for action at the site to which the PA and/or nanomaterial is localized. In exemplary embodiments described herein a RAGE- or ACE-targeting sequence that is part of a PA is used to localize a nanomaterial covalently linked to nitric oxide to a site of intervention of a subject. Embodiments are not limited to such conditions (e.g., pulmonary hypertension, pulmonary injury due to smoke inhalation), targeting moieties (e.g., pulmonary tissue targeting; RAGE ACE, etc.), or therapeutics (e.g., a glutamine, a selectin or leukocyte adhesion molecule inhibitor, a CXCL-1 inhibitor, a perfluorohexane, an inducible nitric oxide synthase (iNOS) inhibitor, a neuronal NOS inhibitor, a peroxynitrite decomposition catalyst, a hydrogen sulfide (H₂S) via a hydrogen sulfide donor, a carvacrol, a nitric oxide, a phosphodiesterase type 5 (PDE5) inhibitor, a tyrosine kinase inhibitor, a peroxisome proliferator-activated receptor-gamma agonist, a statin, or a modulator of LFA-1, ICAM-1, or reactive oxygen species). One of skill in the art will understand how to select and test combinations of therapeutic agents and targeting moieties for prevention and/or treatment of a variety of diseases and conditions. For example, a PA comprising tumor targeting peptides and linked to chemotherapeutics finds use in the treatment of cancer. Likewise, PAs comprising peptides targeting dotting factors and linked to antithrombic agents find use in the treatment or prevention of stroke and/or other cardiovascular conditions. Embodiments find use, for example, in the treatment or prevention of any disease or condition where systemic administration of a therapeutic, followed by localization to a treatment site, is desired.

B. Peptide Amphiphile (PA)-Based Nanomaterials that Target RAGE

In embodiments, a nanotechnology targeted to pulmonary tissue, to eventually serve as a therapeutic delivery platform to treat a pulmonary injury or condition. In embodiments, the pulmonary injury or condition increases the expression of RAGE. In embodiments, the pulmonary disease or condition is pulmonary hypertension. In embodiments, the pulmonary injury or condition is pulmonary injury due to smoke inhalation. Smoke inhalation results in three physiological types of injury: (a) thermal injury predominantly to the upper airway; (b) chemical injury to the upper and lower respiratory tract; and (c) systemic effects of the toxic gases such as CO and CN. In embodiments, the pulmonary injury due to smoke inhalation is pulmonary inflammation or pulmonary fibrosis. In embodiments, the pulmonary injury or condition is cystic fibrosis, chronic obstructive pulmonary disease, acute lung injury and acute respiratory distress syndrome, or lung inflammation due to bacterial infiltration.

Receptor for Advanced Glycation End-Products (RAGE) is a transmembrane protein that is expressed on the basal membrane of healthy pulmonary epithelial cells and aids in regulation of the pulmonary barrier, as well as the differentiation of alveolar epithelial cells.¹ As a key mediator of pulmonary arterial remodeling, RAGE is uniquely expressed within the pulmonary vasculature with high specificity.

Notably, RAGE is involved in propagation of the inflammatory response commonly seen after lung injury such as smoke inhalation injury and current literature demonstrates downregulation of inflammatory cytokines after RAGE inhibition.^{2,3} Similarly, as pulmonary hypertension progresses, increased prevalence of RAGE positively correlates with disease severity.⁴⁸ Patients with pulmonary hypertension demonstrate a six-fold increase in RAGE expression specific to the diseased pulmonary tissue.⁴⁹ This provides promising results for a site-specific targeted therapy after smoke inhalation injury or pulmonary hypertension.

Here, multiple epitopes containing supramolecular nanomaterials self-assembled from peptide amphiphile (PA) molecules are disclosed. The embodiments take advantage of three key features of the pathophysiology of pulmonary injury: increased expression of RAGE. PA nanomaterials are designed to specifically target RAGE in pulmonary tissue, and this nanotechnology is biocompatible. Examples of peptides designed to target RAGE are in Tables 2 and 3. In one embodiment, a peptide capable of targeting an epitope of RAGE is selected from SEQ ID NOs: 1-3 and 9. In one embodiment, a peptide capable of targeting an epitope of RAGE that has at least 95% identity with a sequence selected from SEQ ID NOs: 1-3 and 9. In another embodiment, a peptide capable of targeting an epitope of RAGE that has at least 80%, at least 85%, at least 87%, at least 89%, at least 90%, at least 91%, at least 92%, at least 93%, at least 94%, at least 95%, at least 96%, at least 97%, at least 98%, at least 99%, or 100% identity with a sequence selected from SEQ ID NOs: 1-3.

TABLE 2
PA nanomaterials for targeting injured
lung tissue due to smoke inhalation.

| | | | | | Indications |
|---|---|---|---|---|---|
| | | Pep- | | | for |
| | Se- | tide | Back- | Final | use/ |
| Target | quence | charge | bone | charge | rejection |
| RAGE | LVFF | −2 | C₁₆- | −4 | Significant |
| | AED | | VVAA | | localiza- |
| | (SEQ | | EE | | tion |
| | ID | | (SEQ | | to |
| | NO: | | ID | | injured |
| | 1) | | NO: | | lung |
| | | | 4) | | tissue |
| | | | | | after |
| | | | | | smoke |
| | | | | | inhalation |
| | | | | | injury |
| RAGE | AMVTTAC | −2 | C₁₆- | −4 | No |
| | HEFFE | | VVAAEE | | significant |
| | H | | (SEQ | | localiza- |
| | (SEQ | | ID | | tion |
| | ID | | NO: | | to |
| | NO: | | 4) | | injured |
| | 2) | | | | pulmonary |
| | | | | | tissue |
| | | | | | in |
| | | | | | vivo |
| RAGE | KGVVKA | +3 | C₁₆- | +1 | No |
| | EKSK | | VVAAEE | | significant |
| | (SEQ | | (SEQ | | localiza- |
| | ID | | ID | | tion |
| | NO: | | NO: | | to |
| | 3) | | 4) | | injured |
| | | | | | pulmonary |
| | | | | | tissue |
| | | | | | in |
| | | | | | vivo |

TABLE 3
PA nanomaterials for targeting
pulmonary hypertension.

| | | | | | Indications |
|---|---|---|---|---|---|
| | | Pep- | | | for |
| | Se- | tide | Back- | Final | use/ |
| Target | quence | charge | bone | charge | rejection |
| RAGE | LVFFAED | −2 | C₁₆- | −4 | Significant |
| | (SEQ | | VVAAEE | | localiza- |
| | ID | | (SEQ | | tion |
| | NO: 1) | | ID | | in |
| | | | NO: 4) | | lungs |
| | | | | | with |
| | | | | | pulmonary |
| | | | | | hyper- |
| | | | | | tension |
| RAGE | AMVTTA | −2 | C₁₆- | −4 | No |
| | CHEFF | | VVAAEE | | significant |
| | EH | | (SEQ ID | | localiza- |
| | (SEQ | | NO: 4) | | tion |
| | ID NO: | | | | to |
| | 2) | | | | pulmonary |
| | | | | | tissue |
| | | | | | in |
| | | | | | vivo |
| RAGE | KGVVK | +3 | C₁₆-VV | +1 | No |
| | AEKSK | | AAEE | | significant |
| | (SEQ | | (SEQ ID | | localiza- |
| | ID | | NO: 4) | | tion |
| | NO: 3) | | | | to |
| | | | | | pulmonary |
| | | | | | tissue |
| | | | | | in |
| | | | | | vivo |

C. Peptide Amphiphile (PA)-Based Nanomaterials that Target ACE

In embodiments, a nanotechnology targeted to pulmonary tissue, to eventually serve as a therapeutic delivery platform to treat a pulmonary injury or condition. In embodiments, the pulmonary injury or condition increases the expression of ACE. In embodiments, the pulmonary injury or condition is pulmonary hypertension. In embodiments, the pulmonary injury or condition is pulmonary injury due to smoke inhalation.

Angiotensin converting enzyme (ACE) is a transmembrane protein that converts angiotensin I to angiotensin II with subsequent activation of the renin angiotensin system (RAS), which plays an important role in pulmonary endothelial cell function and vascular remodeling. The enzyme aids in regulation of fluid homeostasis and blood pressure regulation, and expression is significantly higher in the lungs when compared to other tissue. Various experimental pulmonary hypertension models demonstrate a localized and specific increase in ACE antigen in diseased small pulmonary arteries undergoing vascular remodeling.

Furthermore, promising results in animal models show that ACE inhibitors can prevent the development and progression of pulmonary hypertension. Similarly, ACE overexpression in pulmonary tissue occurs after smoke inhalation injury and may even play a role in further propagation of lung injury and edema, which notably holds potential for a targeted drug delivery vehicle after inhalation injury.⁴

Here, multiple epitopes containing supramolecular nanomaterials self-assembled from peptide amphiphile (PA) molecules are disclosed. The embodiments take advantage of three key features of the pathophysiology of pulmonary injury: increased expression of ACE. PA nanomaterials are designed to specifically target ACE in pulmonary tissue, and this nanotechnology is biocompatible. Examples of peptides designed to target ACE are in Tables 4 and 5. In one embodiment, a peptide capable of targeting an epitope of ACE is selected from SEQ ID NOs: 6-8. In one embodiment, a peptide capable of targeting an epitope of ACE that has at least 95% identity with a sequence selected from SEQ ID NOs: 6-8. In another embodiment, a peptide capable of targeting an epitope of ACE that has at least 80%, at least 85%, at least 87%, at least 89%, at least 90%, at least 91%, at least 92%, at least 93%, at least 94%, at least 95%, at least 96%, at least 97%, at least 98%, at least 99%, or 100% identity with a sequence selected from SEQ ID NOs: 6-8.

TABLE 4
PA nanomaterials for targeting injured
lung tissue due to smoke inhalation.

| | | | | | Indications |
|---|---|---|---|---|---|
| | | Pep- | | | for |
| | Se- | tide | Back- | Final | use/ |
| Target | quence | charge | bone | charge | rejection |
| ACE | RYDF | 0 | C₁₆- | −2 | Significant |
| | (SEQ ID | | VVAAEE | | localiza- |
| | NO: 6) | | (SEQ ID | | tion |
| | | | NO: 4) | | to |
| | | | | | injured |
| | | | | | lung |
| | | | | | tissue |
| | | | | | after |
| | | | | | smoke |
| | | | | | inhalation |
| | | | | | injury |
| ACE | TPTQQ | 0 | EEAAW- | −2 | No |
| | (SEQ ID | | K(C₁₂) | | significant |
| | NO: 7) | | (SEQ ID | | localiza- |
| | | | NO: 5) | | tion |
| | | | | | to |
| | | | | | injured |
| | | | | | pulmonary |
| | | | | | tissue |
| | | | | | in |
| | | | | | vivo |

TABLE 5
PA nanomaterials for targeting
pulmonary hypertension.

| | | | | | Indications |
|---|---|---|---|---|---|
| | | Pep- | | | for |
| | Se- | tide | Back- | Final | use/ |
| Target | quence | charge | bone | charge | rejection |
| ACE | GNGSG | +1 | C₁₆- | −1 | Low |
| | YVSR | | VVAAEE | | synthetic |
| | (SEQ ID | | (SEQ ID | | yield |
| | NO: 8) | | NO: 4) | | |
| ACE | RYDF | 0 | C₁₆- | −2 | No |
| | (SEQ ID | | VVAAEE | | significant |
| | NO: 6) | | (SEQ ID | | localiza- |
| | | | NO: 4) | | tion |
| | | | | | to |
| | | | | | pulmonary |
| | | | | | tissue |
| | | | | | in |
| | | | | | vivo |

### IV. Therapeutic Methods

The peptide amphiphile (PA)-based nanomaterials disclosed herein can be used in various methods. For example, they can be used in methods of treating a pulmonary injury or condition in a subject. In embodiments, the pulmonary injury or condition is pulmonary hypertension, pulmonary injury due to smoke inhalation, cystic fibrosis, chronic obstructive pulmonary disease, acute lung injury and acute respiratory distress syndrome, or lung inflammation due to bacterial infiltration.

A method of treating pulmonary hypertension or pulmonary injury due to smoke inhalation in a subject can comprise, for example, administering to the subject a composition comprising one or more peptide amphiphiles comprising: (a) a hydrophobic non-peptidic segment; (b) a β-sheet-forming peptide segment; (c) a charged peptide segment; (d) a targeting moiety, wherein the targeting moiety localizes to RAGE or ACE; and optionally (e) a therapeutic agent; wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

A method of treating pulmonary hypertension or pulmonary injury due to smoke inhalation in a subject can comprise, administering to the subject a composition comprising a self-assembled nanomaterial comprising: a plurality of peptide amphiphiles, wherein said peptide amphiphiles comprise: (a) a hydrophobic non-peptidic segment; (b) a β-sheet-forming peptide segment; (c) a charged peptide segment; (d) a targeting moiety; and optionally (e) a therapeutic agent; wherein the targeting moiety localizes to RAGE or ACE wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

The therapeutic agent for treating pulmonary injury due to smoke inhalation can be, for example, a glutamine, a selectin or leukocyte adhesion molecule inhibitor, a CXCL-1 inhibitor, a perfluorohexane, an inducible nitric oxide synthase (iNOS) inhibitor, a neuronal NOS inhibitor, a peroxynitrite decomposition catalyst, a hydrogen sulfide (H₂S) via a hydrogen sulfide donor, or a carvacrol.

Glutamine—Current in vivo studies observed decreased levels of pro inflammatory cytokines and improved histopathology with decreased pulmonary fibrosis with intravenous administration after smoke inhalation injury in rats.⁵

Inhibition of selectins and leukocyte adhesion molecules—Current in vivo studies have suggested therapeutic benefit of neutralization of these adhesion molecules after smoke inhalation injury. Blockage has been performed using a systemically administered antibody resulting in subsequent inhibition of the inflammatory cascade, vascular permeability, pulmonary edema, and migration of inflammatory cells, although long term systemic effects are unknown.⁶ Since antibodies are too large to attach to PAs, new peptides or small molecules to block these adhesion molecules would need to be developed.

CXCL-1 neutralization—Pulmonary neutrophil infiltration after injury is regulated by a neutrophil chemoattractant, CXCL-1. Presence of neutrophils worsens the progression and pulmonary effects after lung injury and increases release of inflammatory cytokines. Inhibition of this chemoattractant has proven to reduce lung injury and my provide beneficial when administered in a targeted approach after smoke inhalation injury.⁷ Again, since antibodies are too large to attach to PAs, new peptides or small molecules to neutralize CXCL-1 would need to be developed.

Perfluorohexane—this perfluorocarbon with low surface tension and high oxygen carrying capacity has been used in a variety of in vivo models of smoke inhalation injury. Results indicate improvement of lung compliance and inflammation after intratracheal administration, but other effects on lung oxygenation and systemic inflammation remain insignificant or unknown.^{8,9} Targeted administration of this medication may provide additional benefits after inhalation injury.

Nitric oxide synthase inhibition—NO has been investigated in animal models of burn and smoke inhalation injury using sheep and has shown to lower markers of lung tissue injury (interleukin-8, airway pressures, myeloperoxidase activity) after smoke inhalation.¹⁰ Inhibition of inducible nitric oxide synthase (iNOS) has been demonstrated using MEG, BBS-2, BME, and neuronal NOS has been inhibited with the administration of 7-NI. These are potential therapeutics for our targeted drug delivery after smoke inhalation injury.¹⁰⁻¹⁴

Peroxynitrite decomposition catalyst—Peroxynitrite is a powerful and damaging oxidant that results from the combination of the free radicals superoxide and nitric oxide. It contributes to the pulmonary pathological insult resulting from smoke inhalation injury. In vivo models of smoke inhalation injury have used peroxynitrite decomposition catalysts such as W-85, INO-4885, and R-100, resulting in improved oxygenation and edema and decreased pro-inflammatory cytokines after inhalation injury.¹⁵⁻¹⁷

Hydrogen sulfide (H₂S)—H₂S administration using hydrogen sulfide donors (sodium hydrosulfide, sodium sulfide, thiosulfinates, synthetic donors¹⁸) has become another focus of emerging therapy for smoke inhalation injury due to its reported anti-inflammatory effects. Previously, these effects have been demonstrated in models of acute lung injury with notable decreases of pro inflammatory cytokines (IL-6, IL-8) and an increase in anti-inflammatory cytokine IL-10 in treatment groups. These results were also seen in a rat model of smoke inhalation injury.^{19,20}

Carvacrol—This natural phenol has been shown to have anti-inflammatory effects on the lung after injury. It has recently been used in a rat model of smoke inhalation and may prove beneficial if incorporated into our targeted drug delivery system after smoke inhalation injury.²¹

The therapeutic agent for treating pulmonary hypertension can be, for example, a nitric oxide, a phosphodiesterase type 5 (PDE5) inhibitor, a tyrosine kinase inhibitor, a thiazolidinedione (e.g., a peroxisome proliferator-activated receptor-gamma agonist), a statin, or a modulator of LFA-1, ICAM-1, or reactive oxygen species.

Nitric oxide—inhaled nitric oxide is currently the most commonly used vasodilator to treat pulmonary hypertension. However, the inhaled formulary is not effective and intravenous administration is limited by considerable adverse effects due to its non-specific biodistribution. In our laboratory, nitric oxide has been successfully attached to PAs for the targeted treatment of vascular stenosis, thus supporting our novel application in pulmonary hypertension.²³

Sildenafil—Sildenafil is a phosphodiesterase type 5 (PDE5) inhibitor widely used in the management of adult and pediatric pulmonary hypertension. In patients with pulmonary hypertension, PDE inhibitors have been found to improve pulmonary vasodilation, oxygenation, cardiac output, and reduce pulmonary vascular resistance.²⁴⁻²⁷ In vitro studies have successfully co-assembled nanoparticles of sildenafil-loaded polylactide-co-glycolide (PLGA) with promising results demonstrating its feasibility.²⁸

Tyrosine kinase inhibitors—Imatinib is a selective inhibitor of c-Kit and BCR-ABL tyrosine kinase receptors. Although imatinib is commonly used for malignancy, recent evidence in animal models and case reports of adults with severe pulmonary hypertension show that imatinib effectively prevents and/or reduces pulmonary vascular pathophysiology. A randomized controlled trial in adults with medically refractory pulmonary hypertension found decreased pulmonary vascular resistance and increased cardiac output in imatinib-treated patients.²⁹ A recent in vitro and in vivo study synthesized physically stable imatinib mesylate-loaded PLGA nanoparticles that demonstrated sustainable release of the drug.³⁰

Rosiglitazone—Rosiglitazone is an anti-diabetic drug and peroxisome proliferator-activated receptor-gamma agonist that has recently been implicated in pulmonary hypertension pathogenesis. Experimental pulmonary hypertension models show decreased arterial wall thickening and decreased inflammatory mediators, which contribute to vascular remodeling, in the treatment group.^{31,32} Another study designed and created rosiglitazone-based PLGA particles to effectively treat pulmonary hypertension in an animal model.³³

Statins—Inhibitors of 3-hydroxy-3-methylgluaryl-coenzyme reductase, known as statins, exhibit anti-inflammatory, anti-proliferative, and immunosuppressive properties that effectively improve cardiovascular outcomes when used to prevent atherosclerotic disease. Statins improve endothelial cell function and promote vascular relaxation, and given that these pathways are important in the development of pulmonary hypertension, their use as a therapeutic has recently been explored. Experimental pulmonary hypertension rodent models found that simvastatin reduced neointimal hyperplasia, pulmonary arterial pressures, right ventricular hypertrophy, and effectively reversed pulmonary hypertension.^{34,35} Nanoparticles made of PLGA and loaded with pitavastatin were found to be effective in treating pulmonary hypertension in experimental pulmonary hypertension models.^{36,37}

The term “treat” or “treating” refers to both therapeutic treatment and prophylactic or preventative measures, wherein the object is to lessen the symptoms of a pulmonary injury or condition such as pulmonary hypertension or pulmonary injury due to smoke inhalation. Treating may include one or more of directly affecting or curing, suppressing, inhibiting, preventing, reducing the severity of, delaying the onset of, slowing the progression of, stabilizing the progression of, reducing/ameliorating symptoms associated with pulmonary hypertension or pulmonary injury due to smoke inhalation, or a combination thereof.

The term “subject” refers to a mammal (e.g., a human) in need of therapy for, or susceptible to developing, a pulmonary injury or condition such as pulmonary hypertension or pulmonary injury due to smoke inhalation. The term subject also refers to a mammal (e.g., a human) that receives either prophylactic or therapeutic treatment. The subject may include dogs, cats, pigs, cows, sheep, goats, horses, rats, mice, non-human mammals, and humans. The term “subject” does not necessarily exclude an individual that is healthy in all respects and does not have or show signs of a pulmonary injury or condition such as pulmonary hypertension or pulmonary injury due to smoke inhalation.

Pharmaceutical formulations comprising peptide amphiphiles or peptide amphiphile (PA)-based nanomaterials can be prepared for parenteral administration, e.g., bolus or intravenous injection and the like with a pharmaceutically acceptable parenteral vehicle and in a unit dosage injectable form. Peptide amphiphiles or peptide amphiphile (PA)-based nanomaterials are optionally mixed with one or more pharmaceutically acceptable excipients (Remington's Pharmaceutical Sciences (1980) 16^{th} edition, Osol, A. Ed.). Peptide amphiphiles or peptide amphiphile (PA)-based nanomaterials (and any additional therapeutic agent) can be administered by any suitable means, including parenteral, intravenous, intraarterial, intrapulmonary, and the like.

### V. Methods of Making

Methods of making the peptide amphiphile (PA)-based nanomaterials disclosed herein. A method of making peptide amphiphile (PA)-based nanomaterials which target RAGE can comprise, for example, synthesizing PA molecules via solid phase peptide synthesis comprising connecting an RAGE-targeting peptide with a diluent PA backbone; purifying the PA molecules by high-performance liquid chromatography; dissolving targeting PA molecules and the diluent PA in a molar ratio in hexafluoroisopropanol (HFIP); removing the HFIP; and forming the nanomaterials via self-assembly by resuspending the mixture of PA molecules in liquid, such as water or a buffer solution at physiological pH. In embodiments, the liquid is a biological liquid such as blood. In embodiments, the PA molecules are lyophilized. In embodiments, the PA molecules are reconstituted in a liquid prior to administering to a subject.

In embodiments, a RAGE-targeting peptide is covalently incorporated into a diluent PA backbone, for example, C₁₆-VVAAEE (SEQ ID NO: 4).

In embodiments, each targeting PA is further co-assembled with a diluent PA. In embodiments, the optimal parameters for nanomaterial assembly (molar ratios that allow for the best fiber formation, modification of buffer solutions, heating and cooling (e.g., annealing) the PA solutions, or allowing the solutions to sit at room temperature or 4° C. (e.g., aging), etc.), as well as the critical aggregation concentration are determined.

The molar ratio of the targeting PA to the diluent PA (e.g., C₁₆-VVAAEE (SEQ ID NO: 4) or EEAAVVK(C₁₂) (SEQ ID NO: 5)) can be from about 1:100 to about 100:1; or from about 1:50 to about 50:1; or from about 1:10 to about 10:1; about 1:9 to about 9:1; or from about 1:5 to about 5:1; or from about 1:2 to about 2:1; or about 1:1. In embodiments, the molar ratio is about 1:9 to about 9:1. In embodiments, the diluent PA backbone and diluent PA are the same. In embodiments, the diluent PA backbone and diluent PA are different.

Specific embodiments described herein include:

1. A peptide amphiphile comprising: (a) a hydrophobic non-peptidic segment; (b) a β-sheet-forming peptide segment; (c) a charged peptide segment; (d) a targeting moiety, wherein the targeting moiety localizes to pulmonary tissue; wherein the hydrophobic non-peptidic segment is covalently attached to the N-terminus or C-terminus of the β-sheet-forming peptide segment; wherein the β-sheet-forming peptide segment is covalently attached to the targeting moiety; and wherein the charged peptide segment is covalently attached to the targeting moiety.

2. The peptide amphiphile of embodiment 1, wherein said targeting moiety comprises a peptide capable of localizing to an epitope of receptor for advanced glycation end-products (RAGE).

3. The peptide amphiphile of embodiment 2, wherein said peptide comprises a sequence with at least 80% homology to SEQ ID NO: 1.

4. The peptide amphiphile of embodiment 1, wherein said targeting moiety comprises a peptide capable of localizing to an epitope of angiotensin-converting enzyme (ACE).

5. The peptide amphiphile of embodiment 4, wherein said peptide comprises SEQ ID NO: 6.

6. The peptide amphiphile of any one of embodiments 1-5, further comprising a therapeutic agent.

7. The peptide amphiphile of embodiment 6, wherein the therapeutic agent is attached via a covalent bond or a hydrophobic interaction.

8. The peptide amphiphile of embodiment 6, wherein the therapeutic agent is a glutamine, a selectin or leukocyte adhesion molecule inhibitor, a CXCL-1 inhibitor, a perfluorohexane, an inducible nitric oxide synthase (iNOS) inhibitor, a neuronal NOS inhibitor, a peroxynitrite decomposition catalyst, a hydrogen sulfide (H₂S) via a hydrogen sulfide donor, or a carvacrol.

9. The peptide amphiphile of embodiment 8, wherein the selectin or leukocyte adhesion molecule inhibitor or the CXCL-1 inhibitor is a peptide or small molecule.

10. The peptide amphiphile of embodiment 8, wherein the nitric oxide synthase inhibitor is MEG, BBS-2, or BME.

11. The peptide amphiphile of embodiment 8, wherein the neuronal NOS inhibitor is 7-NI.

12. The peptide amphiphile of embodiment 8, wherein the peroxynitrite decomposition catalyst is W-85, INO-4885, or R-100.

13. The peptide amphiphile of embodiment 8, wherein the hydrogen sulfide donors is a sodium hydrosulfide, a sodium sulfide, a thiosulfinate, or a synthetic donor.

14. The peptide amphiphile of embodiment 6, wherein the therapeutic agent is a nitric oxide, a phosphodiesterase type 5 (PDE5) inhibitor, a tyrosine kinase inhibitor, a thiazolidinedione, a statin, or a modulator of LFA-1, ICAM-1, or reactive oxygen species.

15. The peptide amphiphile of embodiment 14, wherein the thiazolidinedione is a peroxisome proliferator-activated receptor-gamma agonist.

16. The peptide amphiphile of embodiment 14, wherein the tyrosine kinase inhibitor is imatinib.

17. The peptide amphiphile of embodiment 1, wherein the C-terminus of the β-sheet-forming peptide segment is covalently attached to the N-terminus of the charged peptide segment; and wherein the C-terminus of the charged peptide segment is covalently attached to the N-terminus of the targeting moiety.

18. A self-assembled nanomaterial comprising:
a plurality of peptide amphiphiles, wherein said peptide amphiphiles comprise:
   (a) a hydrophobic non-peptidic segment;
   (b) a β-sheet-forming peptide segment;
   (c) a charged peptide segment; and
   (d) a targeting moiety, wherein the targeting moiety localizes to receptor for advanced glycation end products (RAGE) or angiotensin-converting enzyme (ACE);
wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

19. The self-assembled nanomaterial of embodiment 18, wherein said targeting moiety comprises a peptide capable of localizing to an epitope of RAGE.

20. The self-assembled nanomaterial of embodiment 19, wherein said peptide comprises a sequence with at least 80% homology to SEQ ID NO: 1.

21. The self-assembled nanomaterial of embodiment 18, wherein said targeting moiety comprises a peptide capable of localizing to an epitope of ACE.

22. The self-assembled nanomaterial of embodiment 21, wherein said peptide comprises SEQ ID NO: 6.

23. The self-assembled nanomaterial of any one of embodiments 18-22, further comprising a therapeutic agent.

24. The self-assembled nanomaterial of embodiment 23, wherein the therapeutic agent is encapsulated in a hydrophobic core of the self-assembled nanofiber.

25. The self-assembled nanomaterial of embodiment 23, wherein the therapeutic agent is a glutamine, a selectin or leukocyte adhesion molecule inhibitor, a CXCL-1 inhibitor, a perfluorohexane, an inducible nitric oxide synthase (iNOS) inhibitor, a neuronal NOS inhibitor, a peroxynitrite decomposition catalyst, a hydrogen sulfide (H₂S) via a hydrogen sulfide donor, or a carvacrol.

26. The self-assembled nanomaterial of embodiment 25, wherein the selectin or leukocyte adhesion molecule inhibitor or the CXCL-1 inhibitor is a peptide or small molecule.

27. The self-assembled nanomaterial of embodiment 25, wherein the nitric oxide synthase inhibitor is MEG, BBS-2, or BME.

28. The self-assembled nanomaterial of embodiment 25, wherein the neuronal

NOS inhibitor is 7-NI.

29. The self-assembled nanomaterial of embodiment 25, wherein the peroxynitrite decomposition catalyst is W-85, INO-4885, or R-100.

30. The self-assembled nanomaterial of embodiment 25, wherein the hydrogen sulfide donors is a sodium hydrosulfide, a sodium sulfide, a thiosulfinate, or a synthetic donor.

31. The self-assembled nanomaterial of embodiment 23, wherein the therapeutic agent is nitric oxide, a phosphodiesterase type 5 inhibitor, a tyrosine kinase inhibitor, a thiazolidinedione, a statin, or a modulator of LFA-1, ICAM-1, or reactive oxygen species.

32. The peptide amphiphile of embodiment 31, wherein the thiazolidinedione is a peroxisome proliferator-activated receptor-gamma agonist.

33. The self-assembled nanomaterial of embodiment 31, wherein the tyrosine kinase inhibitor is imatinib.

34. The self-assembled nanomaterial of any one of embodiments 18-33, wherein the nanomaterial is a nanofiber.

35. The self-assembled nanomaterial of embodiment 34, wherein the nanofiber is about 6 to about 8 nm in diameter.

36. The self-assembled nanomaterial of embodiment 18, wherein the C-terminus of the β-sheet-forming peptide segment is covalently attached to the N-terminus of the charged peptide segment; and wherein the C-terminus of the charged peptide segment is covalently attached to the N-terminus of the targeting moiety.

37. A method of treating a pulmonary injury or condition in a subject comprising, administering to the subject a composition comprising:
one or more peptide amphiphile(s), wherein the peptide amphiphile comprises:
   (a) a hydrophobic non-peptidic segment;
   (b) a β-sheet-forming peptide segment;
   (c) a charged peptide segment;
   (d) a targeting moiety, wherein the targeting moiety localizes to receptor for advanced glycation end products (RAGE) or angiotensin-converting enzyme (ACE); and
   (e) a therapeutic agent;
wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

38. The method of embodiment 37, wherein the pulmonary injury or condition is smoke inhalation injury.

39. The method of embodiment 37, wherein the pulmonary injury or condition is pulmonary hypertension.

40. The method of embodiment 37, wherein the pulmonary injury or condition is cystic fibrosis.

41. The method of embodiment 37, wherein the pulmonary injury or condition is chronic obstructive pulmonary disease.

42. The method of embodiment 37, wherein the composition is administered to the subject via intravascular, inhalational, or intratracheal delivery

43. A method of treating a pulmonary injury or condition in a subject comprising, administering to the subject a composition comprising a self-assembled nanomaterial comprising:
a plurality of peptide amphiphiles, wherein said peptide amphiphiles comprise:
   (a) a hydrophobic non-peptidic segment;
   (b) a β-sheet-forming peptide segment;
   (c) a charged peptide segment;
   (d) a targeting moiety, wherein the targeting moiety localizes to receptor for advanced glycation end products (RAGE) or angiotensin-converting enzyme (ACE); and
   (e) a therapeutic agent;
wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

44. The method of embodiment 43, wherein the pulmonary injury or condition is smoke inhalation injury.

45. The method of embodiment 43, wherein the pulmonary injury or condition is pulmonary hypertension.

46. The method of embodiment 43, wherein the pulmonary injury or condition is cystic fibrosis.

47. The method of embodiment 43, wherein the pulmonary injury or condition is chronic obstructive pulmonary disease.

48. The method of embodiment 43, wherein the composition is administered to the subject via intravascular, inhalational, or intratracheal delivery.

49. A method of delivering a therapeutic agent to pulmonary tissue in a subject comprising, administering to the subject a composition comprising a self-assembled nanomaterial comprising:
a plurality of peptide amphiphiles, wherein said peptide amphiphiles comprise:
   (a) a hydrophobic non-peptidic segment;
   (b) a β-sheet-forming peptide segment;
   (c) a charged peptide segment;
   (d) a targeting moiety, wherein the targeting moiety localizes to receptor for advanced glycation end products (RAGE) or angiotensin-converting enzyme (ACE); and
   (e) a therapeutic agent;
wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

50. A method of making a peptide amphiphile (PA) based nanomaterial which targets receptor for advanced glycation end products (RAGE) or angiotensin-converting enzyme (ACE) comprising:
synthesizing targeting PA molecules via solid phase peptide synthesis comprising contacting a RAGE-targeting peptide with a diluent PA backbone;
purifying the PA molecules;
dissolving targeting PA molecules and with a diluent PA in a molar ratio in a solvent;
removing the solvent; and
forming the nanomaterial via self-assembly by resuspending the mixture of PA molecules in liquid at physiological pH.

51. The method of embodiment 39, wherein the solvent is hexafluoroisopropanol (HFIP).

52. The method of embodiment 39, wherein the liquid is water or a buffer solution.

53. The method of embodiment 39, wherein the nanomaterial is a nanofiber.

54. The method of embodiment 39, wherein the PA molecules are purified by high-performance liquid chromatography.

55. The method of embodiment 39, wherein the molar ratio is about 1:9 to about 9:1.

56. The method of embodiment 39, wherein the diluent PA backbone and the diluent PA are the same.

57. The method of embodiment 39, wherein the diluent PA backbone and the diluent PA are different.

58. The method of embodiment 39, wherein the RAGE or ACE-targeting peptide is connected to the diluent PA backbone by a covalent bond in the resulting targeting PA molecule.

The disclosed subject matter is further described in the following non-limiting Examples. It should be understood that these Examples, while indicating preferred embodiments of the invention, are given by way of illustration only.

### EXAMPLES

### Example 1: Peptide Amphiphile (PA)-Based Nanomaterials for Use in Pulmonary Hypertension

Pulmonary hypertension (PH) is a highly morbid disease without an effective treatment. A systemically administered nanoparticle therapy that specifically targets the pulmonary vasculature was developed and described herein. Angiotensin-converting enzyme (ACE) is highly associated with PH pathogenesis, demonstrating increased expression in the diseased pulmonary vascular endothelium. To target ACE, self-assembled peptide amphiphile (PA) nanofibers are an ideal delivery vehicle, as they are readily modifiable, biocompatible, and can be re-dosed. We hypothesized that ACE-targeted PA nanofibers will localize to the pulmonary vasculature in a mouse model of chronic hypoxia.

Two ACE-targeted amino acid sequences, GNGSGYVSR (“GNG”, SEQ ID NO: 8) and RYDF (SEQ ID NO: 6), were covalently attached to a PA backbone. PAs were synthesized using solid phase peptide synthesis, then purified and characterized by high-performance liquid chromatography paired with mass spectrometry (HPLC-MS). The GNG- (SEQ ID NO: 8) and RYDF- (SEQ ID NO: 6) PA nanofibers were co-assembled using different ratios of backbone PA and fluorescently tagged PA. Conventional transmission electron microscopy (TEM) was used to assess nanofiber formation. Female and male C57BL/6J mice (8-10 weeks old) were exposed to chronic hypoxia (10% FiO₂) for 3 weeks. Control mice were kept at room air (21% FiO₂). To assess in vivo nanofiber localization, targeted nanofiber (10 mg/kg) was administered to control and hypoxic mice via tail vein injection. Lungs were harvested after 30 minutes, and nanofiber fluorescence was quantified.

HPLC-MS confirmed >95% purity of PAs, and TEM confirmed nanofiber formation for both ACE-targeted nanofibers. The mouse PH model was validated by observing pulmonary arterial muscularization per high power field on histology and elevated right ventricular systolic pressure with hemodynamic assessment. ACE immunostaining levels were 5-fold higher in the hypoxic versus control mouse lungs (3106±287 vs. 644±98 AU, n=3, p<0.0001), validating this protein as a useful target. After inducing PH in the mice, targeted nanofibers were injected systemically. Interestingly, the RYDF- (SEQ ID NO: 6) PA nanofiber demonstrated extremely high (78-fold) localization in hypoxic versus control lungs (390±35 vs. 5±2 AU, n=2-4/treatment group, p<0.0001) while the GNG- (SEQ ID NO: 8) PA nanofiber demonstrated no difference in localization between the groups (15±5 vs. 21±5 AU, n=3-4, p=0.39). The pattern of localization of the RYDF (SEQ ID NO: 6)-targeted nanofiber to the pulmonary vasculature had a similar distribution pattern as ACE immunoreactivity on fluorescence microscopy.

An ACE-targeted PA nanofiber was successfully designed and synthesized, and specifically localized to the pulmonary vasculature following intravascular administration in a mouse model of chronic hypoxia. Our findings lay the groundwork for incorporation of a therapeutic into the targeted nanoparticle to effectively mitigate pulmonary hypertension.

### Example 2: Peptide Amphiphile (PA)-Based Nanomaterials for Use in Pulmonary Injury from Smoke Inhalation

Smoke inhalation injury contributes to mortality 20 times higher than seen in burn injury alone. Current treatments, including mechanical ventilation and antibiotics, are supportive and fail to address the underlying lung injury. Using a delivery vehicle of a nanofiber composed of peptide amphiphile (PA) monomers that self-assemble into 3D structures, it is now possible to develop a systemically administered therapy that will localize to the site of injury. The goal of our research was to identify specific proteins for a targeted nanofiber that can be administered after burn inhalation injury and localize to sites of pulmonary injury. We focused on two proteins, ACE and RAGE, which are upregulated in the lung following burn inhalation injury. We hypothesize that nanofibers targeted to ACE or RAGE will localize to the site of pulmonary injury after smoke inhalation.

PAs were synthesized using solid phase peptide synthesis, then purified and characterized by high-performance liquid chromatography paired with mass spectrometry (HPLC-MS). Amino acid sequences targeted to ACE (RYDF, SEQ ID NO: 6) or RAGE (LVFFAED, SEQ ID NO: 1) were incorporated into PA monomers and co assembled with non-targeted and fluorescently labeled PA monomers. Nanofiber formation was confirmed by conventional transmission electron microscopy (TEM). Using a wood chip smoke inhalation injury model, male Sprague Dawley rats (˜350 g) were subjected to 8 minutes of smoke exposure. ACE and RAGE protein levels were evaluated by immunofluorescence. Rats received the targeted nanofiber (7.5 mg) 23 hours after injury and organs were harvested 1 hour later. Nanofiber localization was determined by fluorescent quantification.

HPLC-MS confirmed >95% purity of PAs, and TEM confirmed nanofiber formation.

The wood smoke burn model in rats was validated by histology, neutrophil infiltration, increased bronchial fluid protein, and increased inflammatory cytokines in smoke inhalation vs. control lungs. ACE and RAGE levels were increased in smoke inhalation vs. control lungs (ACE 19598±1748 vs. 5773±565, p<0.001; RAGE 21389±1979 vs. 5183±714, p<0.001). After smoke inhalation and injection of the targeted nanofibers, a 10-fold increase in ACE-targeted nanofiber localization (ACE 1104±65 vs. 114±18, n=5, p<0.001) was found compared to control lung, and a 3-fold increase in RAGE-targeted nanofiber localization (RAGE 623±32 vs. 219±19, n=5-6, p<0.001) compared to control lung. Importantly, minimal localization of non-targeted nanofiber was observed after smoke inhalation injury (394±42, n=5, p<0.001).

This work demonstrates that injectable nanofibers can be synthesized, purified, characterized, and targeted to the site of smoke inhalation injury in a rat model. The ACE-targeted nanofiber represents a novel approach to target and treat lung injury after smoke inhalation and serves as the foundation for incorporation of a therapeutic.

### Example 3: Experimental Design for Designing, Synthesizing, and Characterizing RAGE-Targeted PA Nanofibers and Confirming Localization Specificity In Vitro

Rationale. Nanotechnology-based drug delivery has traditionally been utilized to deliver chemotherapeutics to advantageously accumulate within malignant tumors. Given this rationale, investigators evaluated the use of inhaled and IV nanoparticles for pulmonary disease and successfully demonstrated enhanced accumulation and persistence of nanoparticles within the pulmonary tissue.⁵⁴ However, these studies predominantly rely on liposomes and polymer micelles as delivery vehicles with the vast majority measuring >100 nm and the smallest is 17 nm. Our study is the first to apply PA nanotechnology to specifically target the highly pulmonary-specific RAGE molecule. Self-assembled PA nanofibers are ideal for developing a clinical therapeutic, as they are infinitely modifiable, biocompatible, biodegradable into excreted products, can encapsulate drugs in their hydrophobic core, and can be re-dosed as necessary.⁴⁵ Notably, our laboratory's prior studies highlight the favorably smaller size of our PAs, typically measuring 6-8 nm.^{45,46} This is significant, as nanoparticle size is widely recognized to influence in vivo fate following IV delivery. In particular, smaller sized nanoparticles (≤100 nm) demonstrate longer circulation following IV administration, thereby allowing our therapy to have a much longer effect. To further support this theory, results from our in vivo application of targeted PA nanofibers to prevent restenosis demonstrated a remarkably durable therapeutic effect up to 7 months after administration.⁴⁴ The specific and lasting effect of our intravascular targeted PA nanotechnology is ideally suited to provide a longitudinal therapeutic window, which is necessary to reverse the time-dependent progression of pulmonary hypertension.

Experimental Design. We will identify at least three RAGE-targeting peptide sequences by analyzing the crystal structure of RAGE and mutational binding studies. After identifying peptide sequences that bind to RAGE, PAs containing these peptide sequences will be synthesized via solid phase peptide synthesis and purified by high-pressure liquid chromatography. Non-targeted and scrambled PAs will also be synthesized. Each targeted PA nanofiber will be co-assembled with a non-targeted PA at multiple, specifically defined ratios to establish the optimal ratio necessary for nanofiber assembly and aggregation. Transmission electron microscopy will be performed to confirm and characterize nanofiber formation and size. Binding of the different RAGE-targeted PA and control nanofibers will be assessed in vitro using an enzyme-linked immunosorbent assay (ELISA).

If our targeted PA fails to form nanofibers, we will investigate other targeting sequences, or use different PA backbones with different overall charges. Additionally, if need be, we can add a PEG linker to increase the distance of the targeting peptide from the PA backbone in the event that steric hindrance prevents nanofiber formation. Our targeted PA nanofiber may demonstrate a poor localization to the target protein due to the relatively short length (approximately 10 amino acids) of the incorporated targeting peptide sequence, which limits the surface area available for binding. Fortunately, we have an exhaustive panel of sequences available to target RAGE, which can be further explored if our initial attempts fail to localize sufficiently. In the unlikely event that none of the available sequences localize to the target protein, we are prepared to use different receptors from an extensive list of potential targets that express high specificity for the pulmonary vasculature.

The results of our study are discussed below.

### Example 4: Experimental Design for Evaluate the Localization Specificity of RAGE-Targeted PA Nanofibers to Diseased Pulmonary Tissue Using a Mouse Model of Pulmonary Hypertension In Vivo

Rationale. After engineering our RAGE-targeted PA nanofiber, it will be critical to prove that our targeted therapy can specifically target the diseased pulmonary vasculature involved in pulmonary hypertension with minimal accumulation in other tissues. To this end, we will use the well-established chronic hypoxia murine model to assess in vivo localization. Although no experimental animal model fully encapsulates all of the pulmonary vascular remodeling changes that are reported across the spectrum of human disease, the chronic hypoxia model is advantageous due to its simplicity and reproducibility. It is also the most-studied mouse model for pulmonary hypertension in current literature. Mice with hypoxia-induced pulmonary hypertension reliably develop mild to moderate elevations in right ventricular systolic pressure, mild right ventricular hypertrophy, and pulmonary vascular remodeling with muscularization of precapillary arteries.⁵¹ These histological changes are identical to those seen in a human patient with pulmonary hypertension secondary to a cardiac interatrial septum defect, despite the fact that the latter condition does not induce hypoxia.¹⁶ Importantly, RAGE is similarly upregulated in hypoxia-induced pulmonary hypertension in mice as it is in humans.⁴⁸ Thus, the chronic hypoxia model fortuitously exhibits hallmark pathophysiological alterations, thereby enhancing the likelihood of generating translatable results. It is worth mentioning that a major limitation of this model is its failure to reproduce the obstructive vascular plexiform lesions characteristic of severe pulmonary hypertension.⁵¹ Notably, only one mouse model currently exists that is able to induce these lesions.⁴² However, this genetically modified mouse strain requires significant time and breeding expertise to produce a sufficient number of mice necessary to generate valuable results.

Experimental Design. Eight-week-old male and female C57BL/6 mice will be exposed to chronic hypoxic conditions (10% FiO₂) for 3 weeks to induce pulmonary hypertension. Non-pulmonary hypertension control mice will be exposed to room air (21% FiO₂). To confirm that pulmonary hypertension is established, mice will undergo echocardiogram to assess pulmonary-vascular physiologic parameters including pulmonary vascular resistance and right ventricular dilatation. Echocardiogram is a viable procedure that opportunely allows for serial measurements on the same mouse. Findings will be analyzed by a blinded observer. Afterwards, the chronically hypoxic mice will be injected with the targeted PA nanofibers, scrambled PA nanofiber, or non-targeted PA nanofiber via a single tail vein injection. Of note, these PA nanofibers will be co-assembled with a PA nanofiber containing a fluorescent tag to allow for detection in vivo. Sham animals will receive an injection of saline. Lung samples will be obtained from all five lobes of the murine lungs. Morphometric analysis will be performed to histologically confirm the presence of pulmonary vascular remodeling using routine hematoxylin and eosin staining. Immunohistochemistry will be performed using specific antibodies against RAGE to determine expression of our target protein within the pulmonary vasculature, as well as to confirm co-localization with the targeted PA nanofiber. Specificity and duration of localization to pulmonary tissue and vital organs will be assessed at multiple time intervals (30 minutes, 1 day, 3 days, and 7 days). Optimal concentration, dose (1-10 mg), and co-assembly ratios (10-100%) will be determined. In addition, echocardiogram results will be validated with the use of direct cardiac puncture, a terminal procedure to determine right ventricular systolic pressure. Both methods of measurement are proven to be reliable assessments of pulmonary hypertension.⁵⁶ Difficulties detecting localization of the targeted PA nanofiber to the diseased pulmonary tissue in vivo may be experienced. If this occurs, additional targeting sequences will be evaluated, as the in vitro environment does not always recapitulate the in vivo environment. It may be that the overall charge or polarity of the PA needs to be modified. There may also be difficulty in visualizing the fluorescently labeled PA nanofiber due to technical issues with tissue processing. If this occurs, the fixative solution will be modified to contain a smaller concentration of the embedding material, or we may use an alternate embedding material. If our targeted PA nanofiber demonstrates non-specific affinity in vivo with high accumulation in other organs, other potential targets generated during our original assessment to enhance the reliability of our delivery vehicle will be investigated. Lastly, our targeting peptide sequence may interact with the target protein's activation site when bound, and inadvertently worsen symptoms. To monitor for these adverse effects, cardiopulmonary parameters will be routinely monitored with serial echocardiogram.

The results of our study are discussed below.

### Materials and Methods for Examples 5-9

### Peptide Synthesis and Characterization

Peptide amphiphile (PA) molecule synthesis was performed using 9-fluorenyl methoxycarbonyl solid phase synthesis with Rink Amide 4-methylbenzhydrylamine, or pre-loaded Wang resin (Millipore; Billerica, Mass., USA) on a CEM Liberty Blue automated microwave peptide synthesizer (CEM Corp.; Matthews, N.C., USA). This standardized method was completed as previously described.[^{65,72]} Amino acid sequences specific for our target peptide were incorporated into a non-bioactive PA backbone sequence, which consists of palmitoyl attached to the E₂ sequence (C₁₆-VVAAEE, SEQ ID NO: 4). When used in reverse PA orientation, the E₂ sequence was covalently attached to a lysine carrying the lauroyl chain on its ε-amine (EEAAVV-K-C₁₂, SEQ ID NO: 5). Three sequences designed to target RAGE and 2 sequences designed to target ACE were incorporated into the forward or reverse PA backbone with a di-glycine spacer in-between to produce 5 unique nanofibers: C₁₆-VVAAEE-GG-AMVTTAAHEFFEH-COOH (SEQ ID NO: 17), C₁₆-VVAAEE-GG-KGVVKAEKSK (SEQ ID NO: 10), C₁₆-VVAAEE-GG-LVFFAED (SEQ ID NO: 11), C₁₆-VVAAEE-GG-RYDF (SEQ ID NO: 12), and H₂N-TPTQQ-GG-EEAAVV-K-C₁₂ (SEQ ID NO: 13), respectively (Table 6).

TABLE 6
Characteristics of peptide amphiphiles
targeted to ACE and RAGE.

| Target- | | | Peptide | | |
|---|---|---|---|---|---|
| ing | | | Amphi- | | |
| Se- | Target- | | phile | | Overall |
| quence | ing | | Back- | | Peptide |
| (abbr.) | Se- | Pro- | bone | Back- | Amphi- |
| [SEQ | quence | tein | [SEQ | bone | phile |
| ID] | Charge | Target | ID] | Charge | Charge |
| -RYDF- | 0 | ACE | C₁₆-VV | −2 | −2 |
| CONH2 | | | AAEE- | | |
| [SEQ | | | [SEQ | | |
| ID | | | ID | | |
| NO: | | | NO: | | |
| 6] | | | 4] | | |
| H2N- | 0 | ACE | -EEAA | −2 | −2 |
| TPTQQ- | | | VV-K- | | |
| [SEQ | | | C₁₂ | | |
| ID | | | [SEQ | | |
| NO: | | | ID | | |
| 7] | | | NO: | | |
| | | | 5] | | |
| -AMVTTA | −2 | RAGE | C₁₆-VVA | −2 | −4 |
| CHEFFEH- | | | AEE- | | |
| COOH | | | [SEQ | | |
| (AMVTT) | | | ID | | |
| [SEQ | | | NO: | | |
| ID | | | 4] | | |
| NO: | | | | | |
| 2] | | | | | |
| -KGVVKAE | +3 | RAGE | C₁₆- | −2 | +1 |
| KSK-CONH2 | | | VVAAEE- | | |
| (KGVV) | | | [SEQ | | |
| [SEQ | | | ID | | |
| ID | | | NO: | | |
| NO: | | | 4] | | |
| 3] | | | | | |
| -LVFFAED- | −2 | RAGE | C₁₆- | −2 | −4 |
| CONH2 | | | VVAAEE- | | |
| (LVFF) | | | [SEQ | | |
| [SEQ | | | ID | | |
| ID | | | NO: | | |
| NO: | | | 4] | | |
| 1] | | | | | |

The TPTQQ PA (SEQ ID NO: 7) was synthesized on the reverse backbone (EEAAVV-K-C₁₂; SEQ ID NO: 5) with the aliphatic tail on the C-terminus which left the N-terminus unmodified as the free amine. Synthesized peptides were purified using high-performance liquid chromatography (HPLC) and final purity of lyophilized products was confirmed by liquid chromatography-mass spectrometry (LCMS) as previously described.^{[65]} The purified targeted PAs were then co-assembled with backbone PA and backbone PA labeled with the fluorescent tag 5-carboxytetramethylrhodamine (TAMRA) to allow for identification and enhanced visualization using immunofluorescence microscopy. The ratios of each nanofiber were calculated using molar percent of targeted PA to achieve 25%, 50%, 75%, and 100% targeted epitope. The amount of backbone PA varied depending on the amount of targeted PA, and TAMRA-labeled backbone PA was maintained at a constant 5%. The PAs were then dissolved in hexafluoroisopropanol (HFIP) at 2 mg/mL, mixed at appropriate ratios, and sonicated in a water bath for 15 minutes to allow fibers to mix and completely dissolve in solution. The liquid solution was frozen and HFIP was removed by high vacuum. The remaining nanofiber pellet was then re-suspended in deionized water, aliquoted, and flash frozen in liquid nitrogen. The frozen sample was lyophilized for a minimum of 24 hours until dry and stored at −20° C. until use. To prepare samples for animal use, lyophilized PA was reconstituted in 750 μL Hank's Balanced Salt Solution (HBSS), briefly vortexed, centrifuged, and aspirated into a 27 G needle immediately prior to intravenous administration.

Circular dichroism spectroscopy was used to analyze nanofibers for secondary structure (Chirascan-plus spectrophotometer, Applied Photophysics) over a 0.1 mm path length. Samples were prepared at 2.3 mM (75% RYDF (SEQ ID NO: 6) nanofiber) or 3.1 mM (E₂ backbone nanofiber) in 0.1 M phosphate buffer at 37° C. and scanned from 185-260 nm using 0.3 nm step size and 1.25 second analysis time per data point. For each sample, two scans were averaged and normalized to molar ellipticity per residue.

Conventional transmission electron microscopy was performed using PAs resuspended in HBSS at a final concentration of 0.5 mg/mL. Images were obtained with FEI Tecnai T-12 TEM (ThermoFisher Scientific; Hillsboro, Oreg., USA) at 80 kV with an Onus® 2k×2k CCD camera (Gatan, Inc.; Pleasanton, Calif., USA). Briefly, as previously described, 8 μL samples were spotted on 400-mesh copper grids covered with a thin carbon film and treated with glow discharge for 3 minutes. Following this, samples were washed with deionized water and stained with 2% uranyl acetate for 2-3 minutes. Samples were air-dried before imaging.^{[67]}

Cryogenic transmission electron microscopy samples were prepared by rapid immersion in liquid ethane using a Vitrobot Mark IV (FEI; Hillsboro, Oreg., USA) set to room temperature and 95% humidity. Quantifoil 200 mesh R1.2/1.3 TEM grids (Electron Microscopy Science; Hatfield, Pa., USA) were rendered hydrophobic by glow-discharging for 30 seconds at 15 mA with a PELCO easieGlow (Ted Pella; Redding, Calif., USA). Samples (3 μL) were spotted on grids, incubated in the Vitrobot chamber for 10 seconds, briefly blotted with Whatman 595 filter paper, and then plunged into ethane. Grids were imaged using a 200 kV Thermo Fisher Scientific Talos Arctica G3 and SerialEM software (Boulder Laboratory for 3D Electron Microscopy of Cells; Boulder, Colo., USA) under low-dose conditions. To align the microscope, a cross-gradient TEM grid under parallel illumination conditions at spot size 3 with the 70 μm condenser and 100 μm objective aperture was used. A Ceta CCD camera (FEI; Hillsboro, Oreg., USA) at −3 μm defocus, 92,000× nominal magnification corresponding to a pixel size of 1.6 nm with a total dose of 62 e−/Å² was used to acquire images. Intermediate magnification images were acquired at 85,000× nominal magnification at −15 μm defocus.

Small-angle X-ray scattering (SAXS) and wide-angle X-ray scattering (WAXS) analysis were performed at beamline 5-ID-D of the DuPont-Northwestern-Dow Collaborative Access Team (DND-CAT) Synchrotron Research Center at the Advanced Photon Source (APS), Argonne National Laboratory. PA samples were dissolved at 10 mg/mL in HBSS immediately prior to measuring. Each sample was irradiated for 5 frames of 5 seconds per sample. Data were collected with an X-ray energy at 17 keV (1=0.83 Å). Sample to detector distances were 201.25 mm for SAXS and 8508.4 mm for WAXS. The scattering intensity was recorded in the interval 0.002390<q<4.4578 Å⁻¹. The wave vector q is defined as =(4π/2) sin(θ/2), where θ is the scattering angle. Azimuthal integration of the SAXS pattern to achieve 1D data was achieved using GSAS-II software (UChicago Argonne, LLC) developed at the APS. To prevent damage during beam exposure, samples were oscillated with a syringe pump. HBSS scattering intensity was subtracted from the PA samples using the Irena SAS macro^{[73]} and the resulting plots were fitted using NCNR Analysis macro to a polydisperse core-shell cylinder model.

### Inhalation Injury

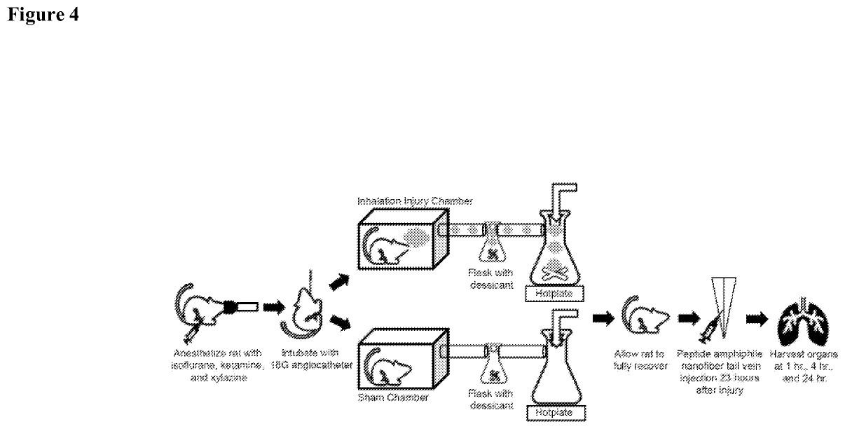

A rat model of smoke inhalation injury was used to induce pulmonary injury to evaluate lung localization of ACE- and RAGE-targeted peptide amphiphile nanofibers. Adult male Sprague Dawley Rats weighing 300-400 g were anesthetized prior to injury with 5% isoflurane for induction and ketamine/xylazine for maintenance (100 mg/kg, 10 mg/kg respectively, intraperitoneal, Patterson Veterinary; Greeley, Colo., USA). After a deep plane of anesthesia was achieved, rats were suspended by incisors on a custom intubation platform and intubated using a small animal laryngoscope (Model LS-2-R, Penn-Century) and an angiocatheter (18 G×1¼ inch, Becton Dickinson and Company; Franklin Lakes, N.J., USA). Rats were then placed in a homemade intubation chamber as previously described^{[74]} and allowed to passively inhale smoke. To maximize smoke exposure and minimize condensation-induced tracheal occlusion, the rat chamber included a DriRite flask placed in sequence between the smoke source and the rat. Smoke was generated from wood shavings in a side arm flask on a hotplate set to 500° C. Rats were exposed to smoke for a total of 8 minutes divided into a 2-minute exposure followed by a 2-minute break in room air, repeated 3 times. Smoke density was maintained at 20-30% by the Ringelmann smoke density chart to achieve visual obstruction and injury as previously described.^{[74]} Following this, rats were extubated, injected with subcutaneous buprenorphine (0.01-0.05 mg/kg), and allowed to fully recover on a heat source. Inhalation injury was immediately confirmed by respiratory changes in the rat and evidence of carbon soot deposition and yellow discoloration in endotracheal tubing. Sham animals underwent the same anesthesia, chamber placement, and recovery, but were allowed to breathe room air. Sacrifice time was determined by experimental time points of the in vivo study (FIG. 4).

### Tissue Harvesting

Rats were sacrificed using the same isoflurane and ketamine/xylazine doses as stated for injury induction and maintenance but followed by bilateral thoracotomies. Bronchoalveolar lavage fluid was collected for analysis. First, rats underwent systemic perfusion via left ventricular inflow and right atrial outflow with approximately 300 mL cold phosphate buffered saline (PBS) followed by approximately 300 mL 2% paraformaldehyde in PBS. Next, tracheal perfusion was performed with a solution composed of two volumes PBS, one volume optimal cutting temperature solution (OCT, Sakura Finetek USA Inc.; Torrance, Calif., USA), and one volume 16% paraformaldehyde (to achieve a final concentration of 4% paraformaldehyde). The lungs were passively inflated with the solution secured at a height of 20 cm until pressure equilibration was achieved. The lung, heart (removed en bloc), liver, spleen, and left kidney were removed and fixed in 2% paraformaldehyde for 1 hour followed by 30% sucrose (in water) for 48 hours.

### Injury Confirmation

Injury confirmation was performed using lung fluid and tissue. Bronchoalveolar lavage fluid was analyzed for protein, inflammatory cells, and inflammatory cytokines, as described previously.^{[74]} Lung tissue was analyzed for wet and dry weight, neutrophil and macrophage infiltration using flow cytometry and immunohistochemistry, and histological evidence of pulmonary injury, as previously described.^{[74]}

### ACE and RAGE Immunostaining

Protein target levels were evaluated using immunofluorescence analysis. Lung tissue was frozen in OCT using liquid nitrogen and sectioned (Cryostar NX70, Thermo Fisher

Scientific Inc.; Waltham, Mass., USA; or Leica CM1950, Leica Biosystems; Buffalo Grove, Ill., USA) or stored at −80° C. Ten to 20 slides, each containing 3 to 4 10-micron sections, were obtained from each lobe. Slides from both sham and injured rats were stained with antibodies against ACE (1:500 dilution; Boster PB9124, Pleasanton, Calif. USA) or RAGE (1:100 dilution; Abcam ab3611, Cambridge, Mass., USA). Slides were incubated in primary antibody overnight at 4° C. Following this, slides were washed and stained with goat anti-rabbit 647 secondary antibody (1:1000 for ACE and 1:500 for RAGE; Fisher Scientific A32733, Rockford, Ill., USA) for 1 hour in the dark, then washed again. Next, Prolong Gold antifade reagent (Life Technologies; Eugene, Oreg., USA) containing 4′,6-diamidino-2-phenylindole (DAPI, Fisher Scientific) was applied to slides, followed by a coverslip. Four evenly distributed images were obtained from each lobe of the lung, excluding the accessory lobe. Immunofluorescence imaging was obtained using a Zeiss Axio Imager.A2 microscope (Hallbergmoos, Germany) and processed using AxioVision x64 4.9.1 software (White Plains, N.Y., USA). Fluorescence quantification was performed using ImageJ Software v1.48 (NIH; Bethesda, Md., USA).

### In Vivo PA Nanofiber Localization

PA nanofiber administration: PA nanofibers were injected into anesthetized rats 23 hours after initial smoke inhalation injury (FIG. 4). Anesthesia was induced with 5% and maintained with 2% isoflurane. Peptide amphiphiles were resuspended in 750 μL HBSS at varying concentrations to achieve a final administered dose of either 5 mg or 7.5 mg. Anesthetized rats were placed in lateral decubitus position and their tails submerged in warm water to allow for vasodilation, which was maintained by heat lamp and heating pad. Next, the PA solution was injected intravenously via tail vein using a 27 G 1 mL syringe. A flash of blood into syringe hub confirmed placement in the vein prior to injection. Rats were returned to cages until they were sacrificed at 24 hours, 28 hours, or 48 hours post-smoke inhalation injury, allowing for evaluation of 1 hour, 4 hour, and 24 hour nanofiber circulation times. Of note, for localization duration and biodstribution studies, rats were anesthetized with ketamine/xylazine without isoflurane to maximize survival and limit possible lung irritation, and subsequent mortality, induced by inhalation anesthesia. Immunofluorescence and lung localization were confirmed both with and without isoflurane.

Tissue imaging and quantification: Fluorescence microscopy was used to image tissue for target protein abundance and in vivo localization of TAMRA-labeled PA. Images were acquired using a Zeiss Axio Imager.A2 microscope with a 20× objective. Four evenly distributed images at 12, 3, 6, and 9 o'clock positions were taken of sections of each organ. Lung images were obtained from each of 4 lobes placed on different slides. Imaged lobes included left lobe, right upper lobe, right middle lobe, and right lower lobe. The accessory lobe was excluded. A total of 16 random and distributed images were taken per animal per organ to allow for standardized quantification and analysis. The HE CY5 filter (Zeiss filter #50) was used to image Alexa 647 using 640 nm excitation and 690 nm emission wavelengths. The CY3 filter (Zeiss filter #43) was used to image TAMRA-labeled peptide amphiphiles with 545 and 605 nm excitation and emission wavelengths, respectively. Autofluorescence of background tissue was measured with 470 nm excitation and 525 nm emission wavelengths (Zeiss filter #38) and appeared green. The DAPI filter (Zeiss filter #49) was used to image cell nuclei at 365 nm excitation and 445 nm emission wavelength. Quantification of target protein or TAMRA-labeled peptide amphiphile nanofiber was performed using area of fluorescence (arbitrary units, AU) measured by ImageJ software. Measurements were obtained using only the channel of interest under constant threshold to eliminate background fluorescence. Quantification results are presented as sham rat versus smoke-injured rat. Images of lung, kidney, spleen, liver, and heart were obtained at 1 hour, 4 hours, and 24 hours after nanofiber injection to assess for localization duration, distribution, and off-target effects.

Statistics: The fluorescence measurement, in AU, was used from each image (4 images per lobe, 16 images per animal) for quantification. Origin Software 2018b (OriginLab; Northampton, Mass., USA) was used for analysis of difference between uninjured lung, injured lung, and off-target organs using a two-way analysis of variance (ANOVA) with Tukey's post hoc test to determine significant differences between groups and Student's t-test when indicated. Results were expressed as mean±the standard error of the mean (SEM). Significance was assumed at p<0.05.

### Example 5: Synthesis of ACE- and RAGE-Targeted Nanofibers for Smoke Inhalation

Specific ACE- and RAGE-targeted PA sequences were identified and amino acids responsible for protein-ligand interaction and binding. First, sequences that target ACE were investigated. ACE is expressed in the lung on pulmonary vascular endothelial cells. ACE inhibition decreases blood pressure and is the therapeutic strategy of this class of hypertensive medications. Because of this clinical result and in an effort to minimize side effects, small natural molecules derived from foods that exhibit the same blood pressure effects have been studied.^{[75]} Lui, et al. specifically investigated inhibitory peptides from a marine invertebrate called Sipuncula (Phascolosoma esculenta), since it contains specific amino acids known to affect ACE inhibition.^{[76,77]} The peptide RYDF (SEQ ID NO: 6) was chosen as this small sequence resulted in non-competitive inhibition of ACE. Molecular docking revealed ACE and RYDF (SEQ ID NO: 6) interactions include only 2 hydrogen bonds and no direct contact with the ACE active site, resulting in minimal effects on blood pressure.^{[77]} Since targeting ACE without causing systemic effects was a goal of this project, this was of specific interest. Investigation of the inhibition mechanism of a peptide generated from the yeast Saccharomyces cerevisiae by ligand receptor docking identified the sequence TPTQQS (SEQ ID NO: 14) as a non-competitive inhibitor of ACE.^{[77]} The only amino acid with direct active site interaction was serine and its removal allowed for continued binding to ACE with the least amount of inhibition (from 73% to 26% with serine removal).^{[78]} Thus, a PA containing the sequence TPTQQ (SEQ ID NO: 7) was generated.

Next, sequences involved with RAGE, a transmembrane protein in the lung, were identified. RAGE was of interest due to its expression on type 1 alveolar epithelial cells, minimal expression in other tissue at healthy baseline, overexpression in injured lungs, and ability to bind multiple ligands.^{[79]} The first sequence was based on the HMGB1 ligand using an inhibitory sequence called RAGE-antagonistic peptide. The 38 amino acid peptide was truncated to highlight the most specific binding region, resulting in the sequence KGVVKAEKSK (“KGVV”, SEQ ID NO: 3).^{[71]} The second ligand was amyloid 13, a protein known to bind to RAGE and play a role in neurotoxicity in Alzheimer's Disease. Specifically, a PA was generated containing the truncated portion of amyloid β, LVFFAED (“LVFF”, SEQ ID NO: 1), that binds the RAGE V domain.^{[80]} The last ligand of interest S100B contains a negatively charged region responsible for binding to the positively charged V domain on RAGE through residues 78-90, AMVTTACHEFFEH (SEQ ID NO: 2).^{[81]} This sequence was previously used to generate a PA with a slight modification in changing the cysteine to an alanine to avoid potential oxidation and purification issues AMVTTAAHEFFEH (AMV, SEQ ID NO: 9). The non-targeted sequence C₁₆-VVAAEE (E₂ backbone, SEQ ID NO: 4) was used as a control.

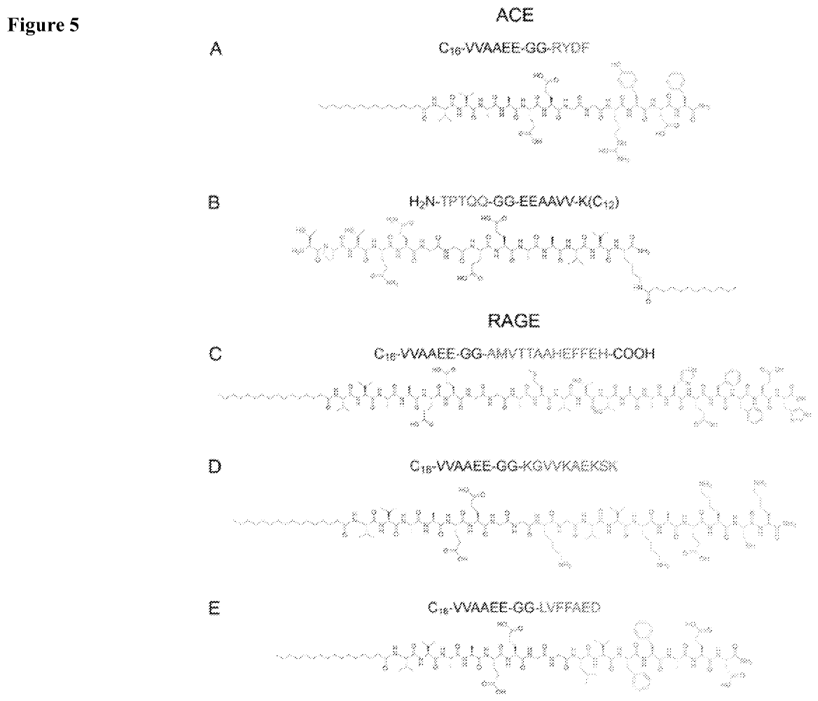

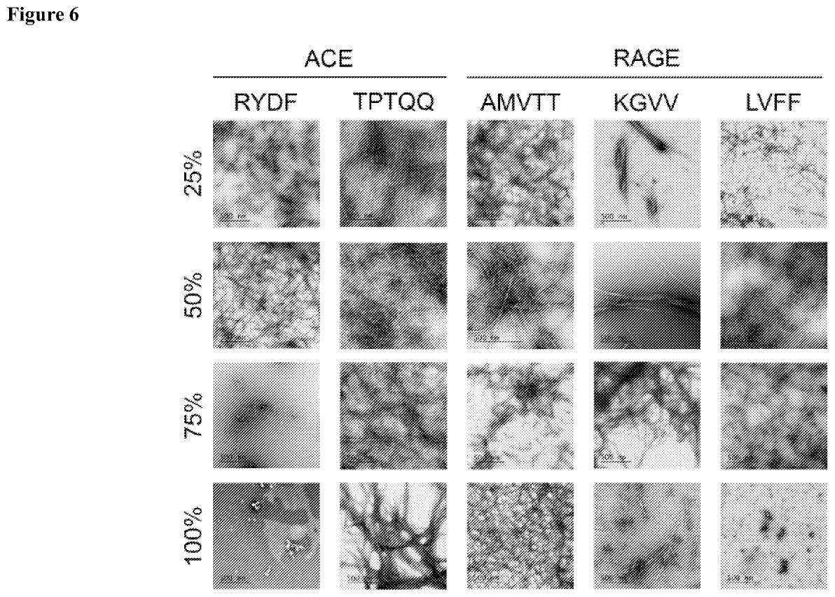

These five sequences (Table 6) were synthesized into peptide amphiphiles (FIG. 5), and then co-assembled at different ratios to optimize fiber formation, confirmed by conventional TEM (FIG. 6).

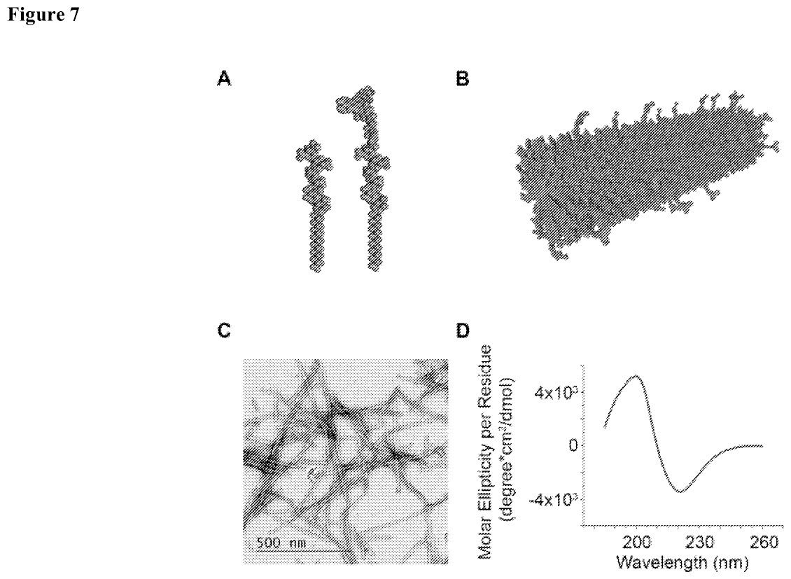

PA ratios were chosen for in vivo work based on fiber quality. PAs were ≥95% pure, as verified by liquid chromatography-mass spectrometry. Non-targeted control (C₁₆-VVAAEE, SEQ ID NO: 4) also showed fiber formation on TEM imaging (FIG. 7).

### Example 6: Smoke Inhalation Injury Confirmed in a Rat Model

Significant histological changes (neutrophil infiltration, proteinaceous debris, vascular congestion), elevation of bronchial fluid protein levels, increased wet-to-dry ratio, elevation of inflammatory cytokines, and infiltration of neutrophils were observed in smoke-injured rat lungs compared to sham controls^{[74]} confirming the smoke inhalation injury.

ACE and RAGE protein levels were elevated after smoke inhalation injury.

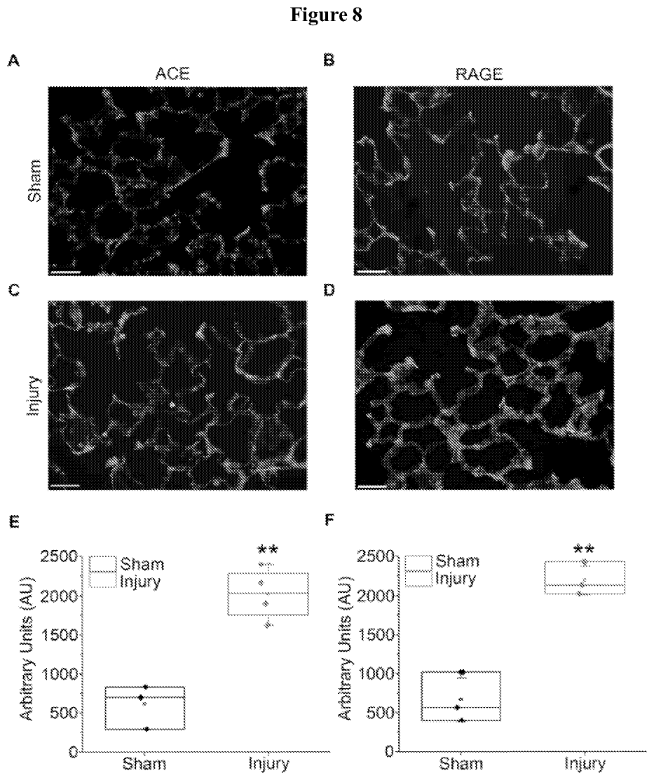

Both ACE (FIGS. 8A and 8C) and RAGE (FIGS. 8B and 8D) levels were increased by almost 4-fold in smoke inhalation versus sham lungs (ACE 19598±1748 vs. 5773±565, p<0.001, FIG. 8E; RAGE 21389±1979 vs. 5183±714, p<0.001, FIG. 8F).

### Example 7: Lung-Targeted Nanofibers Localized to Injured Pulmonary Tissue after Smoke Inhalation Injury

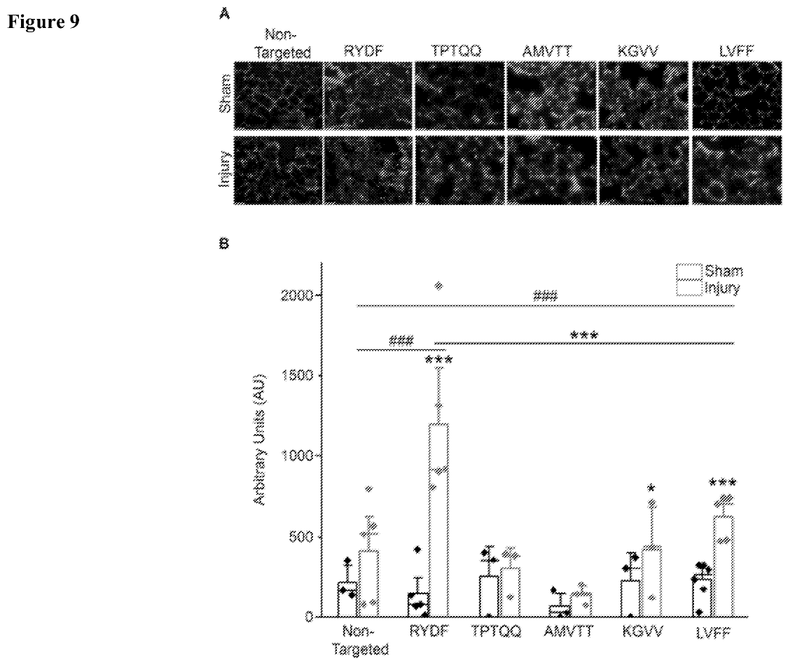

Specific ratios of targeting PA were identified for in vivo work based on fiber formation. Initial tests included injection of the following nanofibers: 50 mole % RYDF (SEQ ID NO: 6), 50 mole % TPTQQ (SEQ ID NO: 7), 50 mole % “AMVTT” (SEQ ID NO: 2), 50 mole % LVFFAED (SEQ ID NO: 1), and 75 mole % “KGVV” (SEQ ID NO: 3) (FIG. 9A). 7.5 mg of nanofiber was administered based on previously tolerated doses in the rat.^{[68]} Lung localization was evaluated using fluorescence microscopy (FIG. 9B).

Interestingly, ACE-targeted RYDF (SEQ ID NO: 6) nanofiber had the highest fluorescence signal in the lung, displaying 10-fold greater localization in smoke-injured lungs versus sham (1104±65 vs. 114±18, n=5-6/group, p<0.001). This was significantly higher than any other targeted nanofiber or non-targeted control (p<0.001). The “LVFF” (SEQ ID NO: 1) RAGE-targeted nanofiber demonstrated 3-fold greater localization to smoke-injured tissue versus sham (623±32 vs. 219±19, n=3/group, p<0.001). The remaining three targeted nanofibers showed minimal lung fluorescence after smoke inhalation injury versus sham animals. Nanofibers containing RAGE-targeted “AMVTT” (SEQ ID NO: 2) (134±20 vs. 58±13, n=3/group) and “KGVV” (SEQ ID NO: 3) (415±54 vs. 215±34, n=3/group), and ACE-targeted TPTQQ (SEQ ID NO: 7) (277±33 vs. 238±32, n=3/group) were not significantly higher than non-targeted controls. Importantly, minimal localization of non-targeted nanofiber was observed after smoke inhalation injury versus sham animals (394±42 vs. 214±23, n=5/6 group). As expected, there was no significant difference in sham animals treated with ACE-targeted nanofibers, RAGE-targeted nanofibers, or non-targeted control nanofibers.

### Example 8: RYDF Nanofiber Optimization by Epitope Ratio and Dosage Allowed for Maximal Lung Localization after Smoke Inhalation Injury

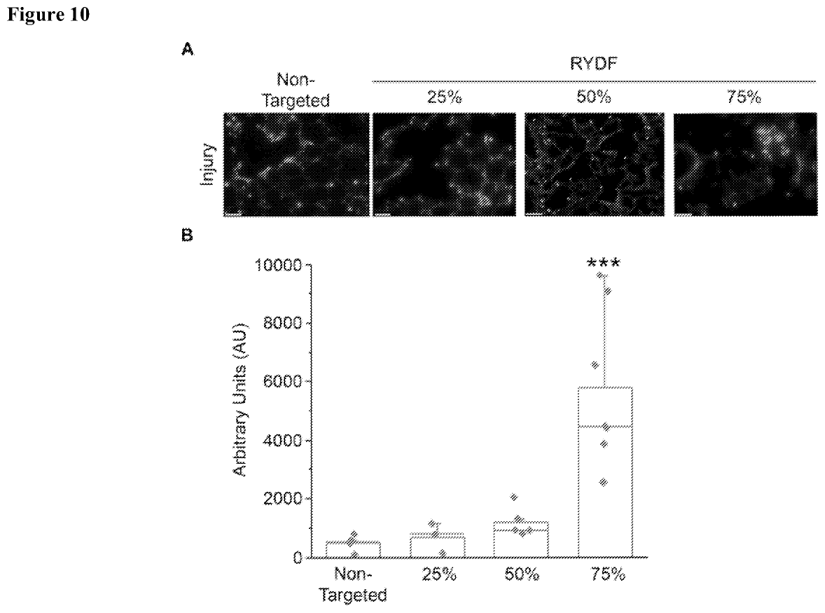

The ACE-targeted RYDF (SEQ ID NO: 6) nanofiber demonstrated the largest fluorescence signal in injured lung tissue versus non-targeted and other targeted nanofibers. This localization was specific to smoke-injured lung tissue as evidenced by minimal lung fluorescence in sham controls injected with RYDF (SEQ ID NO: 6)-targeted nanofiber or non-targeted backbone control (1104±65 vs. 114±18 vs. 214±22, FIG. 9B). Next, different epitope ratios of ACE-targeted RYDF (SEQ ID NO: 6) nanofiber were tested to maximize localization. Rats were injected with 7.5 mg of 25%, 50%, or 75% RYDF (SEQ ID NO: 6) nanofiber and quantified lung fluorescence as previously outlined (FIG. 10A). 100 mole % RYDF (SEQ ID NO: 6) did not form nanofibers and was not tested. A dose-dependent increase in lung localization was observed. The 25 mole % RYDF (SEQ ID NO: 6)-targeted nanofiber demonstrated the smallest amount of lung localization (672±84, n=3), followed by 50 mole % RYDF (SEQ ID NO: 6) nanofiber (1104±65, n=5). Interestingly, more than a 5-fold increase in localization of 75 mole % RYDF (SEQ ID NO: 6) nanofiber versus 50 mole % RYDF (SEQ ID NO: 6) nanofiber (5798±566 vs. 1104±65, n=5-6/group, p<0.001), and a near 15-fold increase versus non-targeted backbone in smoke-injured lungs (5798±566 vs. 394±42, p<0.001, FIG. 10B) was observed.

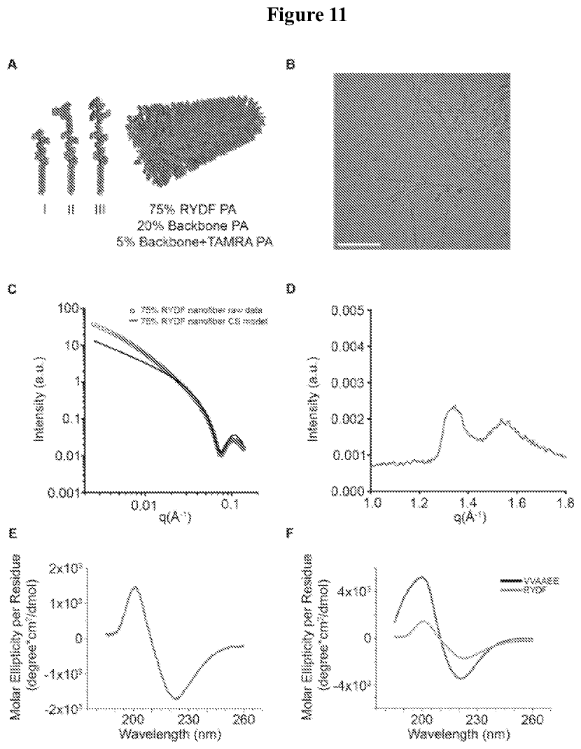

Nanofibers with the 75 mole % RYDF (SEQ ID NO: 6) ratio, which was the most specific to smoke-injured lung tissue, were further characterized. The composition of the 75 mole % RYDF (SEQ ID NO: 6) nanofiber included C₁₆-VVAAEE-GG-RYDF (SEQ ID NO: 12) (75 mole %), C₁₆-VVAAEE (SEQ ID NO: 4) (20 mole %), and C₁₆-VVAAEE-K(TAMRA) (SEQ ID NO: 15) (5 mole %) (FIG. 11A). Cryogenic TEM of fibers in 10% fetal bovine serum confirmed their stability in physiological solutions (FIG. 11B). Small-angle X-ray scattering further confirmed fiber structure (FIG. 11C). Power law slopes for C₁₆-VVAAEE (SEQ ID NO: 4) backbone nanofiber and targeted 75 mole % RYDF (SEQ ID NO: 6) nanofiber were calculated using analysis of scattering intensity versus q in the Guinier region and identified a slope of −1.1 for 75 mole (SEQ ID NO: 6) nanofiber and −1.2 for C₁₆-VVAAEE (SEQ ID NO: 4) nanofiber, indicating cylindrical structure.^{[82]} This method also calculated total radii of 75 mole % RYDF (SEQ ID NO: 6) and C₁₆-VVAAEE (SEQ ID NO: 4) nanofibers of 4.2 nm and 4.62 nm, respectively, by fitting to a cylindrical core-shell model. Wide-angle X-ray scattering identified a peak at q=1.34 Å⁻¹ for both 75 mole % RYDF (SEQ ID NO: 6) nanofiber and C₁₆-VVAAEE (SEQ ID NO: 4) nanofiber, further confirming-sheet formation (FIG. 11D). Circular dichroism spectroscopy also confirmed β-sheet formation of 75 male % RYDF (SEQ ID NO: 6) nanofiber and C₁₆-VVAAEE (SEQ ID NO: 4) backbone nanofiber with positive bands around 195 nm and wide negative bands around 220 nm (FIGS. 11E and 11F).^{[83]}

### Example 9: Lower Doses of RYDF Nanofiber were Detectable in Lungs Up to 24 Hours after Injury

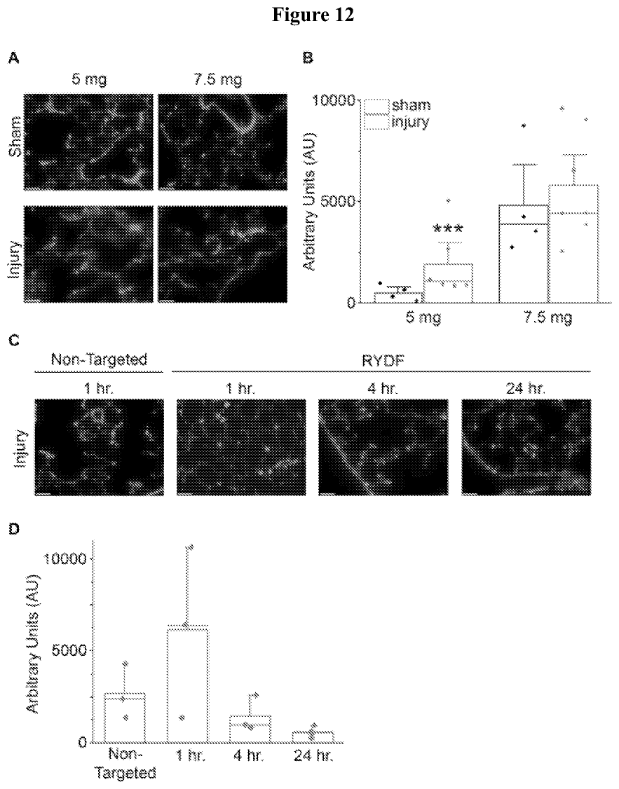

To further optimize the ACE-targeted 75 mole % RYDF (SEQ ID NO: 6) nanofiber, lung localization at different dosages (FIG. 12A) was investigated. Experiments performed using 7.5 mg of 75 mole % RYDF (SEQ ID NO: 6) nanofiber showed high levels of fluorescence in the lung, with no significant difference between injured animals and sham controls (5798±566 vs. 4859±731, p=0.32; FIG. 12B). The high dosage and high epitope percentage may have led to oversaturation of targets and decreased sensitivity. As such, decreasing the dosage restored injured lung specificity. A 7-fold increase in lung localization in injured lung tissue versus sham controls when using 5 mg of 75 mole % RYDF (SEQ ID NO: 6) nanofiber (1822±302 vs. 256±37, p<0.001; FIG. 12B) was observed. This 5 mg targeted localization was also over 4-fold higher than 7.5 mg of the non-targeted control in injured animals (1822±302 vs. 394±42, p<0.001), further supporting specificity of the ACE-targeted RYDF (SEQ ID NO: 6) nanofiber. Thus, the 5 mg dose was selected for localization duration and biodistribution studies.

Animals were sacrificed at different times to measure localization duration and evaluate biodistribution. At 1 hour, the largest amount of 75 mole % RYDF (SEQ ID NO: 6) nanofiber fluorescence in the lung was observed, which was 3-fold higher than the non-targeted nanofiber at the same time (7386±912 vs. 2611±276; FIGS. 12C and 12D).

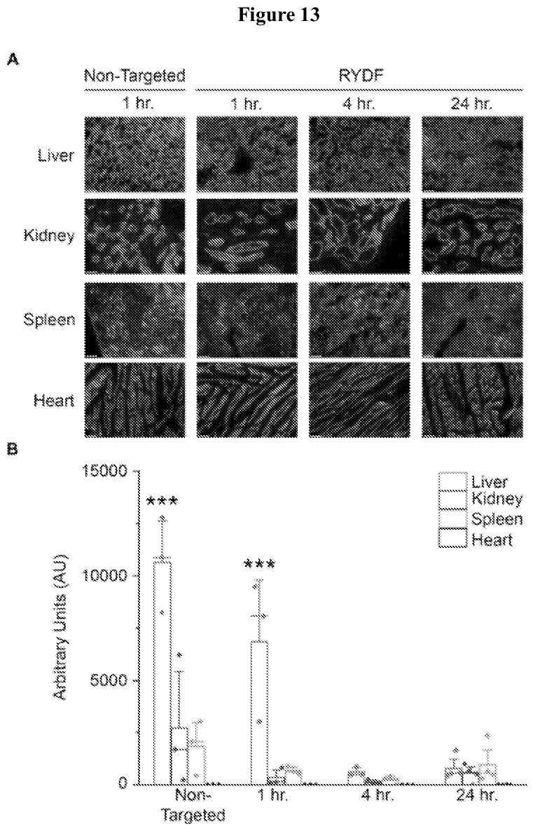

Fluorescence in off-target organs decreased at 4 hours post-injection, but lung localization remained elevated. At 24 hours, nearly all nanofiber had been metabolized and was minimally detectable in all organs (FIGS. 13A and 13B). At 1 hour, nearly equal elevation of liver fluorescence with both targeted and non-targeted nanofibers (6857±1960 vs. 10626±1314; FIG. 13B) is seen due to clearance by the reticuloendothelial system and Kupffer liver cells.^{[84]}

Here, the development of 5 lung-targeted peptide amphiphile nanofibers were evaluated in a rat model of smoke inhalation injury. After confirming increased levels of ACE and RAGE following smoke inhalation injury, the most significant and specific localization of the ACE-targeted RYDF (SEQ ID NO: 6) nanofiber to smoke-injured lung tissue was demonstrated, with the optimized ACE-targeted RYDF (SEQ ID NO: 6) nanofiber exhibiting a 10-fold increase in localization versus sham animals. Regarding biodistribution and safety, fluorescence was observed in the liver as expected due to known processing through the reticuloendothelial system and Kupffer liver cells, but minimal fluorescence was noted in the kidney, spleen, and heart, and no systemic toxicity was noted.^{[68,85]} These data support the synthesis and successful administration of an ACE-targeted nanofiber with localization to smoke-injured lung tissue after inhalation injury. This targeted nanofiber lays the foundation for future incorporation of a therapeutic into a peptide amphiphile platform to treat a disease that is in desperate need of novel therapeutics.

The angiotensin-converting enzyme is of particular interest to pulmonary research, as it is located on pulmonary vascular endothelial cells. ACE-1 participates in the renin-angiotensin system by conversion of angiotensin I to angiotensin II, which leads to vasoconstriction and fluid homeostasis.^{[86]} ACE-2 counteracts this effect by degradation of angiotensin II. Studies have shown a positive correlation between activation of the renin-angiotensin system and acute lung injury in animal models of respiratory distress syndrome.^{[69]} Interestingly, studies have also shown a potential protective mechanism in the activation of ACE-2,^{[87,88]} which is especially germane to our study considering the upregulation of both ACE-1 and ACE-2 after smoke inhalation injury.^{[69]} The RYDF sequence (SEQ ID NO: 6) was based on the catalytic domain of ACE-1, but potential effects on homologous ACE-2 are unknown. As the ACE-targeted nanofiber disclosed herein localizes to the lung, it may also have some therapeutic effect through localization-induced activation or inhibition of these enzymes.

Clinically, nanotechnology has been explored in a variety of lung-specific diseases. It has not only been used for treatment, but also to detect biomarkers of injury and for pulmonary imaging.^{[62,89]} The drug delivery aspect has been particularly useful in complicated diseases like lung cancer, chronic pulmonary disease, and infection.^{[60,85]} Aerosolized targeted drug delivery has been explored in lung disease such as asthma, cystic fibrosis, and pulmonary arterial hypertension. This administration route bypasses the alveolar capillary barrier membrane, but may also initiate pulmonary inflammatory response and exacerbate injury. This toxicity is partly particle-size dependent, with large size correlating with decreased toxicity. This is particularly problematic as larger particles limit deep lung distribution and can result in obstruction.^{[90,91}] Lipid encapsulated chemotherapy agents have allowed for intravenous administration of higher drug doses via nanocarrier.^{[60,92}] This administration route has been described using passive delivery to the lung, taking advantage of the ability of nanoparticles to accumulate in cells of the reticuloendothelial system, namely liver, spleen, and lung; however, results are mixed. Animal models have shown that passive administration leads to variable tissue accumulation amounts, specifically noting lung biodistribution to fall anywhere from 0.09% to 33%.^{[85]} Other lung-targeted mechanisms have been suggested. Akaska, et al. injected antibody-targeted chemotherapy into mice with Lewis lung carcinoma but found minimal localization to cancerous tissue.^{[93]} Similarly, Muro, et al. attempted to deliver antioxidants to pulmonary endothelial cell adhesion molecules using antibody targeting to mitigate inflammation and found promising results.^{[94]} Despite this there is still a clear need for specific lung-targeted, nanomaterial-based drug delivery.

Unfortunately, these advances have not been translated to smoke inhalation injury. Currently, there is one nanotherapeutic that has been tested in an animal model of smoke inhalation injury. Carvalho, et al. administered aerosolized carvacrol encased in lipid nanoparticles to rats after smoke inhalation.^{[95]} They reported decreased levels of malondialdehyde in treated groups versus controls, as well as decreased signs of oxidative stress and injury in proximal lung tissue; however, similar results were observed in groups treated with oxygen alone.^{[95]} Although these results are encouraging, concerns regarding this administration route and subsequent efficacy remain. It is difficult to determine the exact dosage of medication administered via aerosolized liquids, which introduces variability and limits reproducibility. Furthermore, inhaled particles may be deposited in larger, more proximal airways, limiting distribution and potential distal alveolar effect. This is supported by histological changes in the Carvalho, et al. study, as most were observed in the proximal airway.^{[95]} Other emerging therapeutics, such as stem cell therapy, immunomodulation, and peroxynitrite decomposition have been tested in animal models of smoke inhalation injury, but do not yet include nanotechnology.^{[96]} Although promising, none of these studies include targeted drug delivery or incorporation into nanoparticles to maximize therapeutic benefit after smoke inhalation injury. This reinforces the need for our targeted peptide amphiphile nanofiber and supports clinical translatability into the field of smoke inhalation injury.

Exact co-localization of TAMRA-labeled RYDF (SEQ ID NO: 6) nanofiber with ACE via immunostaining has not yet been demonstrated, which may be attributable to PA lung localization and blockage of antibody targets. However, similar fluorescence patterns between our targeted nanofiber and ACE levels were observed, supporting nanofiber localization.

Disclosed herein is an ACE-targeted nanofiber that successfully localizes to injured lung tissue after inhaled smoke exposure in a rat model. Two of 5 systemically administered ACE- and RAGE-targeted nanofibers (RYDF (SEQ ID NO: 6) and “LVFF” (SEQ ID NO: 1), respectively) localized to smoke-injured lungs, with the ACE-targeted nanofiber displaying a 10-fold increase in localization to injured lung tissue. This targeted nanofiber provides a foundation for the development of a novel therapeutic to treat smoke inhalation injury.

### Materials and Methods for Examples 10-18

PA nanofiber preparation. ACE- and RAGE-targeted peptides were synthesized using standard 9-fluorenyl methoxycarbonyl (Fmoc) SPPS on Rink amide 4-methylbenzhydrylamine, or pre-loaded Wang resin (Millipore, Billerica, Mass.), as previously described.^{[141]} The forward backbone PA, VVAAEE (SEQ ID NO: 4), consists of palmitoyl attached to VVAAEE (C₁₆-VVAAEE, SEQ ID NO: 4) while the second backbone PA, EEAAVV-K-C₁₂ (SEQ ID NO: 5), is oriented in the reverse order and attached to a lysine bearing the fatty acid chain on its ε-amine (EEAAVV-K-C₁₂, (SEQ ID NO: 5). In the synthesis of the “GNG” (SEQ ID NO: 8) PA, RYDF (SEQ ID NO: 6) PA, “KGVV” (SEQ ID NO: 3) PA, and “LVFF” (SEQ ID NO: 1) PAs, the forward backbone PA sequence was separated from the targeting epitope with a glycine-glycine linkage to facilitate nanofiber self-assembly and to enhance accessibility to the epitope while displayed on surface of the fiber. This produced C₁₆-VVAAEE-GG-GNGSGYVSR-COOH (SEQ ID NO: 16), C₁₆-VVAAEE-GG-RYDF-CONH₂ (SEQ ID NO: 12), C₁₆-VVAAEE-GG-KGVVKAEKSK-CONH₂ (SEQ ID NO: 10), C₁₆-VVAAEE-GG-AMVTTAAHEFFEH-COOH (SEQ ID NO: 17) and C₁₆-VVAAEE-GG-LVFFAED-CONH₂ (SEQ ID NO: 11), respectively. The “TPTQ” (SEQ ID NO: 7) PA was the only sequence synthesized on the reverse backbone PA, EEAAVV-K-C₁₂ (SEQ ID NO: 5), to expose the N-terminus of this epitope that was determined to be important for targeting. Also incorporating a glycine-glycine linker, the final sequence with free N-terminus was H₂N-TPTQQ-GG-EEAAVV-K-C₁₂ (SEQ ID NO: 13). To enhance visualization, the 5-carboxytetramethylrhodamine (TAMRA) fluorophore was attached to the ε-amine of an added lysine residue in the forward backbone PA and to the N-terminal amine of the reverse backbone PA to yield C₁₆-VVAAEE-K(TAMRA) (SEQ ID NO: 15) and (TAMRA)-EEAAVV-K-C₁₂ (SEQ ID NO: 5) respectively. All PAs were purified by HPLC and characterized by HPLC-MS, as previously described.^{[167]}

ACE- and RAGE-targeted PA nanofibers were co-assembled at different molar ratios containing the targeted PA, non-targeted backbone PA, and fluorophore-labeled backbone PA (5%). All targeted PA co-assemblies contained 25 mole %, 50 mole %, 75 mole %, or 100 mole % targeted epitope PA. For co-assembly, the individual PAs were dissolved in 1,1,1,3,3,3-hexafluoro-2-propanol (HFIP) and water bath sonicated for 15 minutes. Samples were frozen in liquid nitrogen, and HFIP was removed under high vacuum until dry. Samples were reconstituted in deionized water, lyophilized, and stored at −20° C. until use. On the day of injection, PAs were kept at room temperature and dissolved in HBSS at 1 mg/mL, 2 mg/mL, or 4 mg/mL depending on the dose (5, 10, or 20 mg/kg, respectively).

Circular dichroism (CD) spectroscopy was performed on a Chirascan-plus spectrophotometer (Applied Photophysics) over a 0.1 mm path length. Samples were prepared at 1 mg/mL in 0.1M phosphate-buffered saline (PBS) and scanned at 37° C. from 185-260 nm, 0.3 nm step size, and 1.25 second analysis time per data point. Two scans were averaged for each sample and data were normalized to molar ellipticity per residue (degree*cm²/dmol).

Conventional TEM images were obtained with a FEI Tecnai T-12 TEM (Thermo Fisher Scientific; Hillsboro, Oreg.) at 80 kV with a Gatan Onus® 2k×2k CCD camera (Gatan, Inc.; Pleasanton, Calif.). PAs were reconstituted at 1 mM in HBSS, and samples (8 μL) were pipetted onto copper supports covered with thin carbon foil 400 mesh followed by 2-minute treatment with glow discharge. Samples were rinsed with deionized water and then stained with 2% uranyl acetate prior to TEM imaging.

Cryogenic TEM cryogrids were prepared by rapid immersion in liquid ethane using a Vitrobot Mark IV (FEI, Hillsboro, Oreg.) set to room temperature and 95% humidity. Quantifoil 200 mesh R1.2/1.3 TEM grids (Electron Microscopy Science, Hatfield, Pa.) were rendered hydrophobic by glow-discharging for 30 seconds at 15 mA with a PELCO easieGlow (Ted Pella, Redding, Calif.). Before cryo-plunging, samples (3 μL) were applied to the carbon side of the TEM grid and then incubated in the Vitrobot chamber for 10 seconds. Samples were blotted for 2-4 seconds with Whatman 595 filter paper. Cryogrids were imaged with a 200 kV Thermo Fisher Scientific Talos Arctica G3 under low-dose conditions using the software platform SerialEM (Boulder Laboratory for 3D Electron Microscopy of Cells, Boulder, Colo.). The microscope was aligned using a cross-gradient TEM grid under parallel illumination conditions at spot size 3 with the 70 μm condenser and 100 μm objective aperture. Images were acquired with a Ceta CCD camera (FEI, Hillsboro, Oreg.) at −3 μm defocus, 92,000× nominal magnification corresponding to a pixel size of 1.6 nm with a total dose of 62 e−/Å².

X-ray scattering analysis (SAXS/WAXS) experiments were performed at beamline 5-ID-D of the DuPont-Northwestern-Dow Collaborative Access Team (DND-CAT) Synchrotron Research Center at the Advanced Photon Source (APS), Argonne National Laboratory. PA samples were dissolved at 10 mg/mL in HBSS immediately prior to measuring. Each sample was irradiated for 5 frames of 5 seconds per sample. Data was collected with an X-ray energy at 17 keV (1=0.83 Å). Sample to detector distances were as follows: 201.25 mm for SAXS, 1014.2 mm for MAXS, and 8508.4 mm for WAXS. The scattering intensity was recorded in the interval 0.002390<q<4.4578 Å⁻¹. The wave vector q is defined as =(4π/λ) sin(θ/2), where θ is the scattering angle. Azimuthal integration of the SAXS pattern to achieve 1D data was achieved using GSAS-II software (UChicago Argonne, LLC) developed at the APS. Samples were oscillated with a syringe pump during exposure to prevent beam damage. The scattering intensities of HBSS were subtracted from the PA samples using the Irena SAS macro^{[168]}, and the resulting plots were fitted using the NCNR Analysis macro to a polydisperse core-shell cylinder model on Igor Pro software (version. 8.03, WaveMetrics, Lake Oswego, Oreg.).

Animal model. Eight- to 10-week-old male and female C57BL/6 mice were purchased from Jackson Laboratory (Bar Harbor, Me.) and Charles River Laboratory (Wilmington, Mass.). Following a 1-week acclimation period, mice underwent echocardiography to determine baseline cardiac function. Mice were exposed to chronic normobaric, hypoxic conditions (10% FiO₂) with placement inside a plexiglass ventilated chamber. Oxygen levels were controlled by ProOx 360 controller (BioSpherix, Parish, N.Y.) to achieve appropriate hypoxic levels. Normoxic controls were kept in room air (21% FiO₂). After 3 weeks, animals underwent follow up echocardiography, cardiac catheterization, and then tissues were harvested for morphometry and histology analysis.

Echocardiography. Mice were anesthetized with inhaled isoflurane (1-3%) and placed on a temperature-controlled heating pad. Pulmonary arterial flow was assessed with the VisualSonics Vevo 2100 (Toronto, ON, Canada) ultrasound system and a 40-MHz MicroScan solid state transducer (VisualSonics Model MS550D), as has been previously described by our lab.^{[169]} Briefly, parasternal short axis views at the aortic level in 2D mode were obtained to visualize the right ventricle and tricuspid valve. On color Doppler mode, blood flow through the tricuspid valve was recorded to calculate PVR via the equation PVR=maximum velocity of tricuspid regurgitation divided by the VTI of the right ventricular outflow tract (RVOT).^{[170]} A modified parasternal long axis view in the color Doppler mode visualized blood flow through the RVOT and pulmonary artery. This was used to measure VTI and peak velocity of the pulmonary artery. PAT was measured as the time elapsed between the waveform to the point of pulmonary artery peak velocity and flow through RVOT, and served an estimate for the mean pulmonary arterial pressure.^{[171]} Echocardiograms were performed by a pediatric cardiac sonographer who was blinded to hypoxic treatment. Off-line data analysis was performed with Vevo Lab 3.1.1 software (Toronto, ON, Canada) by a cardiologist who was also blinded to hypoxic treatment.

Hemodynamic measurements. RVSP was measured with right heart catheterization. The abdominal cavity of the anesthetized mouse was entered, and the silhouette of the heart was visualized through the intact diaphragm. The right ventricle was punctured through the diaphragm using a 25 G needle attached to a water-filled pressure transducer (ADInstruments, Colorado Springs, Colo.). RVSP waveform was recorded (30 to 60 seconds) and analyzed using a PowerLab data acquisition module and LabChart 8.0 software (ADInstruments).

Tissue processing. Lung intratracheal fixation was performed with 1:1:1 volume mixture of 4% paraformaldehyde (PFA), PBS, and optimal cutting temperature compound (OCT) at 20 cm of H₂O. Systemic vasculature was fixed with 2% PFA via the left ventricle. Lungs and heart were removed en bloc. The kidney, heart, spleen, and left lobe of the liver were also harvested. Tissues were fixed in 2% PFA for 2 hours and transferred to 20% sucrose for 24-48 hours at 4° C. Samples were embedded in OCT, frozen with liquid nitrogen, and sectioned on a CryoStar NX70 cryostat (ThermoScientific) in 10-μm steps.

Histologic assessment. Lung sections were stained with hematoxylin and eosin according to standard protocol. Digital images were obtained with light microscopy (Zeiss Axio Imager.A2, Halbergmoos, Germany). For each mouse, ten high-power field (20× objective) images representative of right and left lung lobes were randomly selected and used for analysis. Small (25-75 μm) pulmonary resistance vessels were categorized as fully muscularized (complete medial muscle layer), partially muscularized (partial medial muscle layer), or non-muscularized (no visible muscle layer) and manually counted using ImageJ software by an observer blinded to hypoxic exposure. The sum number of each vessel type was analyzed per mouse.

To assess vessel muscularization, slides were stained with anti-SMC-α actin antibody (1:250 dilution; Dako M0851, Carpinteria, Calif.). To assess target protein levels, slides were stained with anti-ACE antibody (1:500 dilution; Boster PB9124, Pleasanton, Calif.) or anti-RAGE antibody (1:500 dilution; Abcam ab3611, Cambridge, Mass.). To determine colocalization of “LVFF” (SEQ ID NO: 1) nanofiber to RAGE, slides of lungs from mice injected with the targeted nanofiber were stained with the anti-RAGE antibody at the same dilution. Slides were incubated overnight at 4° C., washed, and then stained with goat anti-rabbit 647 secondary antibody (1:1000 dilution for α-actin and ACE, and 1:500 dilution for RAGE; Fisher Scientific A32733, Rockford, Ill.) for 1 hour in the dark at room temperature. After washing, coverslips were mounted with ProLong Gold antifade mountant with DAPI (Fisher Scientific). All images were visualized with a Zeiss Axio Imager.A2 microscope and AxioVision x64 4.9.1 software (White Plains, N.Y.). Four high-power field (20× objective) images per lung lobe, excluding the posterior accessory lobe, were randomly selected per mouse. Fluorescence intensity was calculated using ImageJ software.

PA nanofiber injections. A catheter made of polyethylene-10 (PE-10) tubing (Fisher Scientific) was inserted into the tail vein of the anesthetized mouse and placement was confirmed on blood return. PA nanofibers reconstituted in HBSS were injected. Following a normal saline flush, catheters were removed and manual pressure was applied to achieve hemostasis. Animals were awoken and resumed normal activity until the time of sacrifice at 30 minutes, 4 hours, and 24 hours after injection for respective experiments. Animals sacrificed at 24 hours were returned to hypoxic conditions following injection.

Tissue processing for LSFM. Similar to frozen sections, tissues underwent systemic fixation with 2% PFA via the left ventricle of the heart. Lungs were inflated with 1 mL of warm (40-45° C.) 1% agarose via tracheal cannulation. Heart and lungs were removed en bloc. The kidney, heart, spleen, and left lobe of the liver were harvested. Tissues were fixed in 2% PFA for 2 hours, and kept in PBS overnight at 4° C. Using a modified iDisco protocol, tissue was dehydrated with serial methanol (MeOH, Fisher Scientific A452-4) incubations at varying concentrations (20%, 40%, 60%, 80%, and 100%) for one hour each. Samples were transferred to another 100% MeOH solution overnight. For clearing, samples were placed in a solution of 66% dichloromethane (DCM, VWR BDH1113-4LG, Radnor, Pa.) and 33% MeOH for 3 hours on a shaker at room temperature. After serial washes in 100% DCM (15 minutes×3), samples were transferred into 100% dibenzyl ether (33630-1L, Sigma-Aldrich, St. Louis, Mo.) until imaging. All steps were performed in a light-protected environment.

Light sheet fluorescence microscopy. Images were obtained with the Ultramicroscope II light-sheet system (LaVision BioTec, Bielefeld, Germany) and Imspector Pro software. Two laser channels were used: 488 nm laser for intrinsic lung autofluorescence (green) and 561 nm “OBIS” laser for the TAMRA fluorophore-labeled PA (red). Standardization of image acquisition from the 561 nm laser, which had the material of interest, included: zoom magnification (x0.63), exposure time (10 ms), laser power (40%), light-sheet numerical aperture illumination (0.031), light-sheet width (100%), and light-sheet horizontal focus position (both left and right lasers). This produced 3D reconstruction with images at 7-μm resolution. Quantification of the 561 nm laser was standardized by using a threshold fluorescence intensity level. To determine the threshold, the absolute fluorescence intensity from ten hypoxic mice treated with targeted nanofibers was measured. Based on overall average, a conservative intensity threshold that captured true nanofiber fluorescence was established. Four equal-sized quadrants of the left lobe of the lung were quantified. Due to its much larger size, the left lobe was used to represent the entire lung after confirming equal distribution in all lobes. Spatial graph data provided: volume of fluorescence (mm³), number of individual fluorescence objects, fluorescence intensity (arbitrary units [a.u.]), and lung volume (mm³) per quadrant. Fluorescence levels were calculated by the volume of fluorescence (mm³) divided by the lung volume (mm³). Off-target organ localization was evaluated in liver, kidney, and heart in similar fashion. The spleen was not visualized with LSFM due to significant artifact resulting from retained pigmented blood that could not be adequately cleared with current protocols. Data were analyzed using Imaris software (Oxford Instruments, Concord, Mass.).

Urinary fluorescence. Urine samples were collected from treated and non-treated hypoxic mice at the time of sacrifice (30 minutes, 4 hours, and 24 hours after injection). Samples stored at −80° C. were thawed to room temperature and diluted 1:20 using HBSS. For standard curve preparation and to determine absolute amount of nanofiber present in urine sample, stock amounts of lyophilized product of the same co-assembled nanofiber injected into the mouse were diluted in HBSS ranging from 1.5 to 600 μg. Samples and standards were plated onto black, clear-bottom, glass tissue culture 96-well plates (Greiner bio-one, Monroe, N.C.) in duplicate at 50 μL per well. The fluorescence of TAMRA (co-assembled with targeting “LVFF” (SEQ ID NO: 1) PA, non-targeted VVAAEE (SEQ ID NO: 4) backbone PA, and TAMRA-labeled backbone PA at 25%, 70%, and 5% molar concentrations, respectively) was measured using a single fluorescence intensity read step at 546/20 nm for excitation and 579/20 nm for emission with Gen5 software on a Cytation 5 plate reader (BioTek Instruments, Winooski, Vt.). Fluorescence emission values for treated animals were fitted to a dose-response curve calculated from standards using OriginPro 2018b software (Northampton, Mass.). Percent of excreted fluorescence was calculated by dividing the amount of fluorescence in the urine (mg) by the fluorescent nanofiber dose administered (mg).

Statistical analysis. Data analysis was performed with OriginPro 2018b. Normality of data distribution was assessed by Shapiro-Wilk normality test. Depending on distribution, nonparametric tests (Mann Whitney test for unpaired sample and Kruskal-Wallis test for analysis of groups with Bonferroni correction for post hoc analysis when appropriate) or parametric tests (paired and 2-sample Student's t-test with Welch's correction if variances were unequal, and one-way or two-way analysis of variance with Tukey's post hoc test to determine differences between groups) were used. P<0.05 was considered statistically significant. Data are expressed as mean±standard error of mean (SEM).

HPLC/Mass spectrometry. Liquid chromatography mass spectrometry (LCMS) of purified PAs was obtained on an Agilent 6520 QTOF LCMS using a gradient of water (buffer A) and acetonitrile (buffer B) both containing 0.1% ammonium hydroxide. Samples were injected onto a Phenomenex Gemini C18 column (150×1 mm) using a gradient starting at 5% buffer B, 0-5 min, followed by a linear gradient up to 95% buffer B from 5-30 min. Purity was determined by integration of the eluting peaks at 220 nm and the identity confirmed by ESI-MS.

### Example 10: Design and Synthesis of PA Nanofibers for Intravenous Delivery to Mice with Pulmonary Hypertension

Three targeting epitopes for ACE and RAGE, respectively, were selected based on crystal structures and complementary biochemical analyses examining the interaction between the proteins and their ligands. For ACE, two amino acid targeting sequences were chosen, GNGSGYVSR (GNG, SEQ ID NO: 8) and RYDF (SEQ ID NO: 6), originally purified from Sipuncula (Phascolosoma esculenta) and confirmed as non-competitive binding inhibitors of ACE.^{[122,123]} Some studies demonstrate that inhibition of ACE in pulmonary hypertension improves cardiopulmonary remodeling and hemodynamics,^{[124-126]} while others do not show a significant benefit.^{[127,128]} Given this potential therapeutic advantage, we sought to use it as a target for our delivery vehicle. Both targeting peptides interact with hydrophobic and hydrophilic residues on ACE via a combination of electrostatic interactions, hydrogen bonds, and van der Waals forces. The third targeting sequence, TPTQQ (TPTQ, SEQ ID NO: 7), is a non-competitive ACE inhibitor isolated from hydrolyzed yeast (Saccharomyces cerevisiae).^{[129,130]} This sequence is largely hydrophilic and mutational analyses confirm the critical role “TPTQ” (SEQ ID NO: 7) residues play in binding to ACE.^{[129]}

Three RAGE-targeted peptide sequences were selected that mimic naturally occurring RAGE-binding ligands. The extracellular domain of RAGE, composed of the immunoglobulin domains V, C1, and C2, is theorized to be a pattern recognition receptor that identifies negatively charged and hydrophobic ligands.^{[131]} The first targeting sequence, AMVTTAAHEFFEH (AMV, SEQ ID NO: 9), contains residues 78 to 90 from the S100B ligand.^{[132]} Mapping studies demonstrate that negatively charged residues on its interfacing surface interact with the positively charged surface of the RAGE V domain.^{[132,133]} The second targeting sequence was from the high mobility group box 1 (HMGB1) ligand, which binds and activates RAGE via its highly acidic C-terminus.^{[134]} However, previous in vitro and in vivo studies show that a peptide fragment of the C-terminus of HMGB1, which excludes the acidic tail, binds and antagonizes RAGE to inhibit tumor cell migration^{[135]} and inflammatory processes^{[136]} in the lung. As these antagonistic properties are favorable for our disease model, this region of the ligand was chosen as the targeting sequence. Residues 173 to 182, KGVVKAEKSK (KGVV, SEQ ID NO: 3) were chosen, given their resemblance to the N terminus of 5100 proteins across species.^{[135]} The final targeting sequence contained residues 17 to 23, LVFFAED (LVFF, SEQ ID NO: 1), from the amyloid beta (Aβ) ligand.^{[137,138]} This peptide interacts with the V domain of RAGE via electrostatic and hydrophobic bonds in a sequence-specific manner.^{[138]} Two non-targeted peptides were also used as fiber-forming PA backbone components, C₁₆-VVAAEE (VVAAEE, SEQ ID NO: 4) and EEAAVV-K-C₁₂ (SEQ ID NO: 5), and were used as controls.

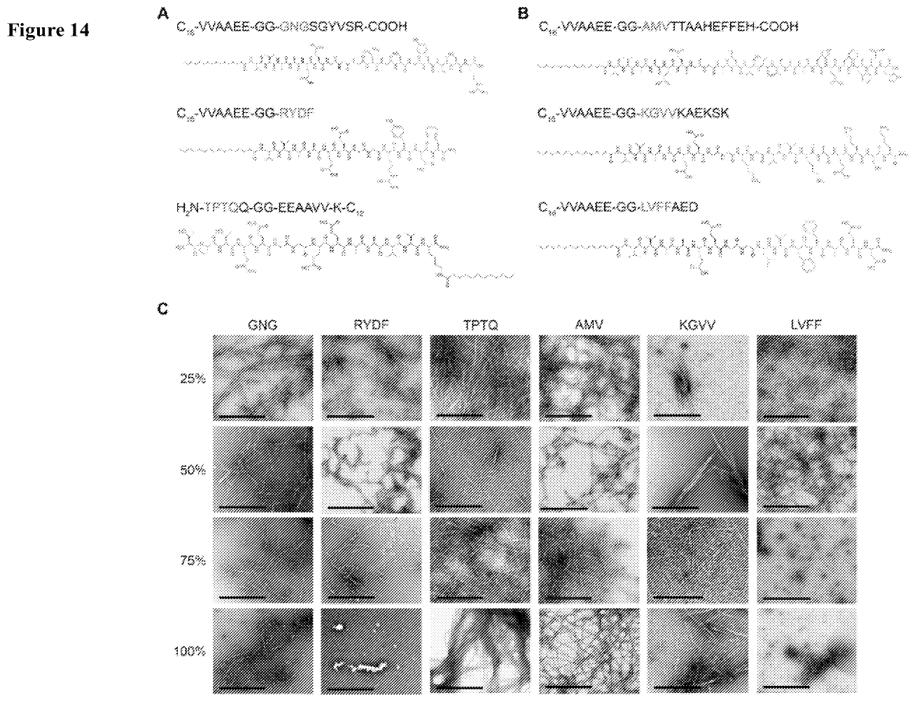

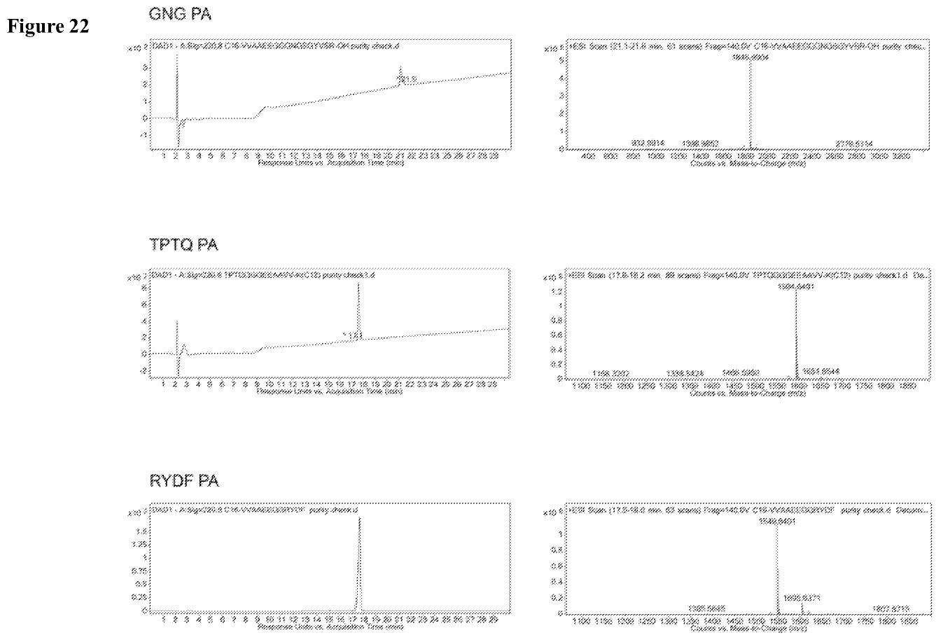

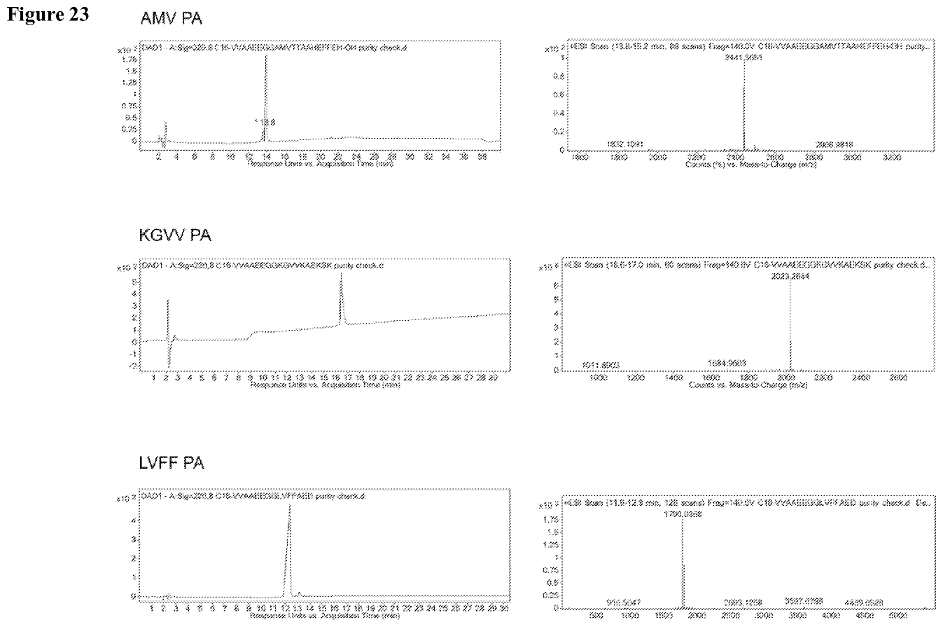

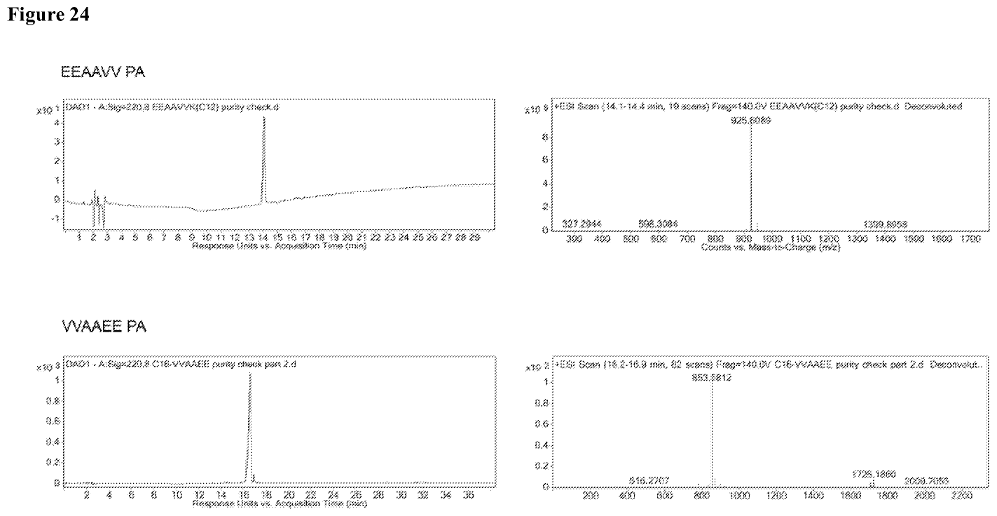

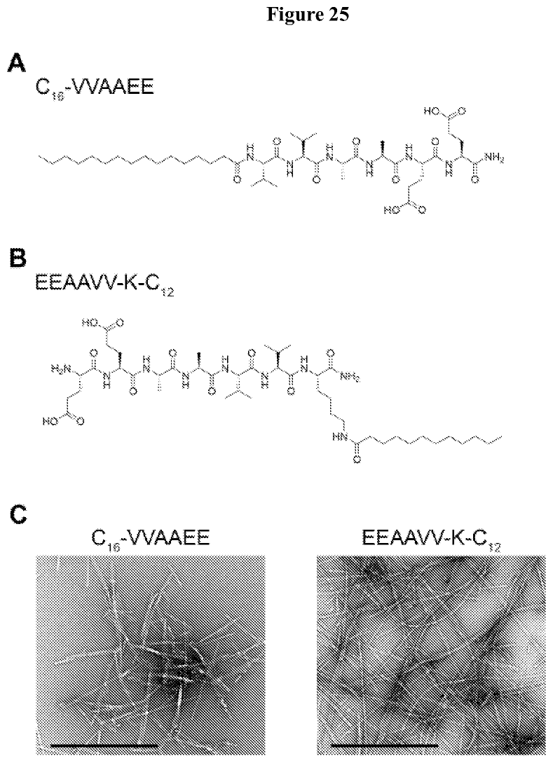

The aforementioned peptide sequences were incorporated to produce three ACE- (FIG. 14A) and three RAGE- (FIG. 14B) targeted PAs. PAs synthesized via solid phase peptide synthesis (SPPS) were purified to ≥95%, as verified by high-performance liquid chromatography and mass spectrometry (HPLC/MS, FIGS. 22-24). To enable nanofiber formation, ACE- and RAGE-targeted PAs (Table 7) were co-assembled with non-targeted backbone PA at varying molar ratios, containing 25% to 100% targeted PA. Conventional transmission electron microscopy (TEM) was performed to visualize and characterize nanofibers (FIG. 14C). Fiber formation varied across both targeting epitopes and molar ratios, highlighting the influence co-assembly has on nanoparticle structure and morphology. Similar results have been reported by our laboratories during development of targeted PA nanofibers for atherosclerotic disease.^{[139-141]} Accordingly, the co-assembly ratio that best induced fiber formation for each targeted PA was identified. In general, the highest concentration of targeted epitope that produced well-formed fibers was selected for further investigation. However, in cases where multiple co-assembly ratios produced comparable fibers, the mid-ratio co-assembly was selected to best reflect the range of effective fiber-forming co-assembly options. Ultimately, the following nanofibers with the indicated mole percentage of targeted epitope were chosen: 50% RYDF (SEQ ID NO: 6), 50% “TPTQ” (SEQ ID NO: 7), 50% “AMV” (SEQ ID NO: 9), 75% “KGVV” (SEQ ID NO: 3), and 50% “LVFF” (SEQ ID NO: 1). GNG (SEQ ID NO: 8) PA was not tested in vivo due to low yield during synthesis. Both non-targeted backbone PAs, VVAAEE (SEQ ID NO: 4) and EEAAVV-K-C₁₂ (SEQ ID NO: 5), produced well-formed fibers (FIG. 25).

TABLE 7
Protein and chemical properties of ACE-
and RAGE-targeted PA molecules.

| | ACE-targeted | | | RAGE-targeted | | |
|---|---|---|---|---|---|---|
| | PAs | | | PAs | | |
| Se- | GNG | RY | TPT | AMVTT | KGVV | LVFF |
| quence | SGY | DF | QQ | AAHEF | KAEK | AED |
| | VSR | | | FEH | SK | |
| (abbr.) | (GNG) | (RYDF) | (TPTQ) | (AMV) | (KGW) | (LVFF) |
| [SEQ | [SEQ | [SEQ | [SEQ | [SEQ | [SEQ | [SEQ |
| ID] | ID | ID | ID | ID | ID | ID |
| | NO: | NO: | NO: | NO: | NO: | NO: |
| | 8] | 6] | 7] | 9] | 3] | 1] |
| Target | ACE | ACE | ACE | RAGE | RAGE | RAGE |
| Target | +1 | 0 | 0 | −2 | +3 | −2 |
| peptide | | | | | | |
| charge | | | | | | |
| Non- | C₁₆- | C₁₆- | EEA | C₁₆-VVA | C₁₆- | C₁₆- |
| targeted | VVA | VVA | AVV- | AEE | VVA | VVA |
| back- | AEE | AEE | K-C₁₂ | | AEE | AEE |
| bone | [SEQ | [SEQ | [SEQ | [SEQ | [SEQ | [SEQ |
| [SEQ | ID | ID | ID | ID | ID | ID |
| ID] | NO: | NO: | NO: | NO: | NO: | NO: |
| | 4] | 4] | 5] | 4] | 4] | 4] |
| Direction | For- | For- | Re- | For- | For- | For- |
| | ward | ward | verse | ward | ward | ward |
| Backbone | −2 | −2 | −2 | −2 | −2 | −2 |
| charge | | | | | | |
| Final | −1 | −2 | −2 | −4 | +1 | −4 |
| PA | | | | | | |
| charge | | | | | | |

### Example 11: Generation of Pulmonary Hypertension in CBL57/6 Mice Exposed to Chronic Hypoxia

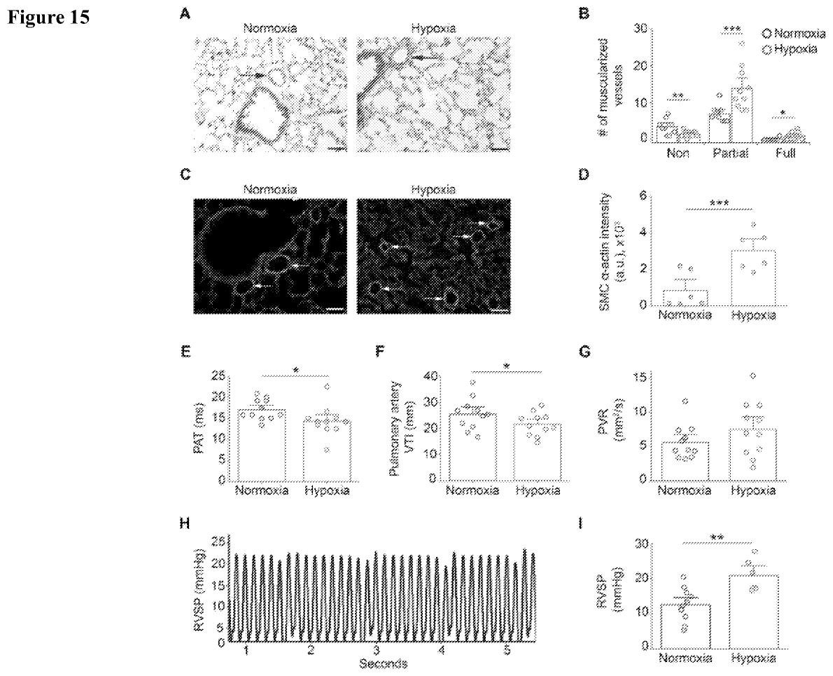

To demonstrate the utility of ACE- and RAGE-targeted nanofibers for targeting the lung, our animal model for in vivo analysis was confirmed. The well-established chronic hypoxia-induced pulmonary hypertension mouse model was used, as it reliably reproduces mild to moderate elevations in right ventricular systolic pressure (RVSP) and vessel wall hypermuscularization.^{[142,143]} In this study, 8- to 10-week-old CBL57/6 male and female mice were maintained in normobaric, hypoxic (10% FiO₂) conditions for three weeks to induce pulmonary hypertension. Normoxic controls were exposed to room air (21% FiO₂). Histological analysis evaluated vessel muscularization in normoxic versus hypoxic lungs (FIG. 15A). Quantitatively, there were fewer small (25-75 μm) non-muscularized pulmonary resistance vessels, while conversely more partially and fully muscularized vessels in hypoxic versus normoxic lungs (FIG. 15B). Immunofluorescence staining of SMC α-actin in pulmonary vessels showed significantly greater fluorescence in hypoxic mice (FIG. 15C-D), indicative of hypermuscularization. The increased density of muscularized vessels is consistent with vascular remodeling related to pulmonary hypertension. Cardiopulmonary hemodynamics are also key determinants of disease severity. Transthoracic echocardiography showed evidence of elevated pulmonary arterial pressure in hypoxic compared to normoxic mice, as noted by decreased pulmonary acceleration time (PAT, FIG. 15E), decreased pulmonary acceleration velocity time index (VTI, FIG. 15F), and increased pulmonary vascular resistance (PVR, FIG. 15G). Cardiac catheterization was performed, which is the gold standard for evaluation and diagnosis of pulmonary hypertension. RVSP (FIG. 15H) was significantly higher in hypoxic mice (FIG. 15I).

### Example 12: Upregulated ACE and RAGE Pulmonary Levels in Pulmonary Hypertension

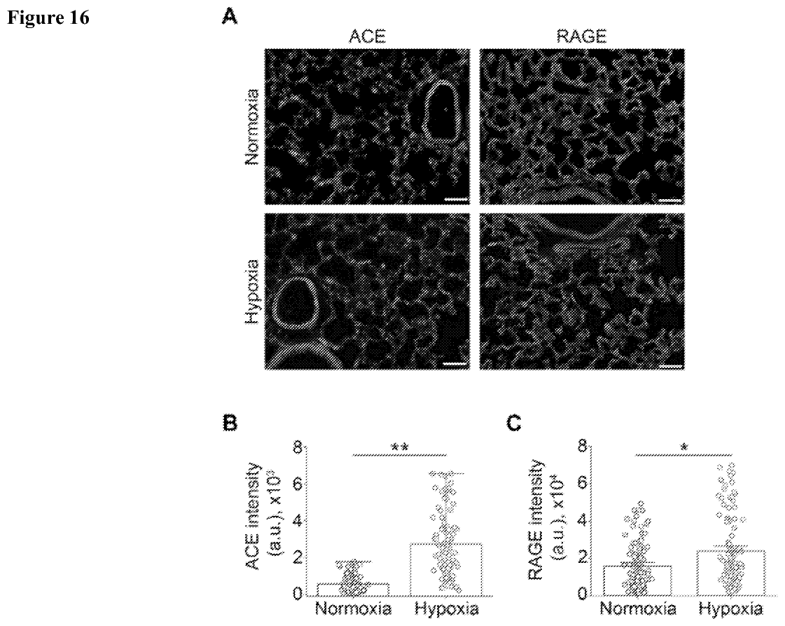

ACE and RAGE were confirmed to be upregulated in the chronic hypoxia-induced pulmonary hypertension mouse model. Immunofluorescence staining was performed to quantify fluorescence intensity of the target proteins in the lung (FIG. 16A). Significantly increased pulmonary levels of ACE (FIG. 16B) and RAGE (FIG. 16C) were found in hypoxic compared to normoxic mice. Interestingly, in diseased lungs, RAGE fluorescence intensity (FIG. 16C) was nearly 9-fold higher than ACE (FIG. 16B). Moreover, RAGE levels in normoxic controls (FIG. 16C) were almost 6-fold higher than ACE levels in the hypoxia-exposed group (FIG. 16B). These findings are consistent with the literature,^{[144]} which demonstrates high basal levels of RAGE in healthy human lungs.

### Example 13: RAGE-Targeted LVFF Nanofibers Localize to Lungs with Pulmonary Hypertension

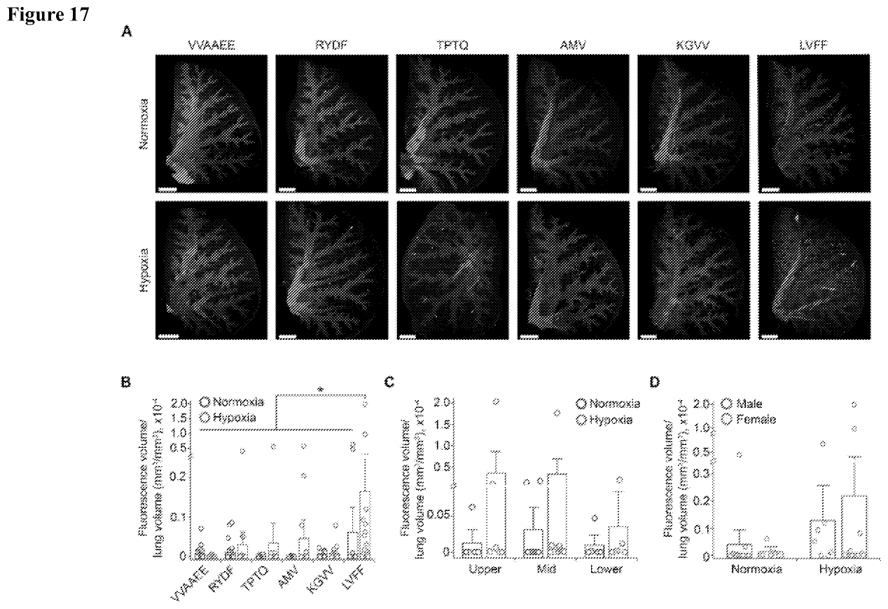

To compare nanofiber lung localization in vivo, normoxic and hypoxic mice were injected with fluorophore-labeled ACE- and RAGE-targeted PA nanofibers via tail vein catheters. Lungs were imaged and quantified with three-dimensional (3D) light sheet fluorescence microscopy (LSFM, FIG. 17A); PA nanofiber levels were measured as fluorescence volume per lung volume (mm³/mm³). LSFM was used because it provides fast, large-scale, high quality 3D images that allow efficient evaluation of nanoparticle distribution within entire organs. This is preferable to other quantitative techniques including, conventional fluorescence microscopy and in vivo imaging systems (IVIS) imaging, which are limited by low resolution, poor sensitivity, inconsistent tissue distribution, and incomplete specimen sampling.

Overall, the RAGE-targeted “LVFF” (SEQ ID NO: 1) nanofiber had significantly greater lung localization compared to all other ACE- and RAGE-targeted nanofibers (FIG. 17B). Amongst hypoxic mice, there was a 3.5-fold difference between the “LVFF” (SEQ ID NO: 1) nanofiber and the next most efficient RAGE-targeted nanofiber, “AMV” (SEQ ID NO: 9). When compared to “TPTQ” (SEQ ID NO: 7), which was the most efficient ACE-targeted nanofiber, the “LVFF” (SEQ ID NO: 1) nanofiber had 5 times more fluorescence volume detected in the diseased lungs (FIG. 17B). This reflects the difference in ACE and RAGE pulmonary levels that we observed on immunofluorescence analysis (FIG. 16).

There was a striking 300-fold increase in nanofiber fluorescence within hypoxic lungs injected with targeted “LVFF” (SEQ ID NO: 1) nanofibers compared to non-targeted VVAAEE (SEQ ID NO: 4) nanofibers (1.7×10⁻⁵±1.0×10⁻⁵ mm³/mm³ vs. 5.2×10⁻⁸±3.3×10⁻⁸ mm³/mm³, respectively; FIG. 17B). No other ACE- or RAGE-targeted nanofiber demonstrated a significant difference compared to the non-targeted VVAAEE (SEQ ID NO: 4) nanofiber in hypoxic lungs (FIG. 17B). The EEAAVV-K-C₁₂ (SEQ ID NO: 5) backbone PA was not evaluated as it was only incorporated in the “TPTQ” (SEQ ID NO: 7) nanofiber, which itself had minimal localization detected on LSFM. Since the remaining nanomaterials, including the “LVFF” (SEQ ID NO: 1) PA, were constituted with the VVAAEE (SEQ ID NO: 4) backbone PA, only this non-targeted nanofiber was included in the analysis.

In normoxic conditions, there was no significant difference in nanofiber localization between any of the targeted nanofibers and the non-targeted VVAAEE (SEQ ID NO: 4) nanofiber (FIG. 17B). Although not significant, normoxic mice that received the “LVFF” (SEQ ID NO: 1) nanofiber showed more lung localization compared to other ACE- and RAGE-targeted nanofibers. This, too, mimics our immunofluorescence findings of high RAGE levels at baseline, which is consistent with the literature.^{[145]}

To verify nanofiber specificity for diseased lungs, lung localization between nanofiber-treated hypoxic mice and normoxic controls were compared. The “LVFF” (SEQ ID NO: 1) nanofiber had significantly greater localization in diseased versus non-diseased lungs (FIG. 17B). Indeed, of all the targeted nanofibers, the “LVFF” (SEQ ID NO: 1) nanofiber was the only one to demonstrate a significant difference in lung localization between hypoxic and normoxic mice. This suggests that targeting by the “LVFF” (SEQ ID NO: 1) nanofiber was specific for molecular markers involved in hypoxia-induced pulmonary hypertension. In both normoxic and hypoxic mice, “LVFF” (SEQ ID NO: 1) nanofiber fluorescence was observed throughout the entire lung (FIG. 17C). There was no difference in fluorescence volumes between males or females (FIG. 17D).

### Example 14: Colocalization of LVFF Nanofiber to RAGE in Lung with Pulmonary Hypertension

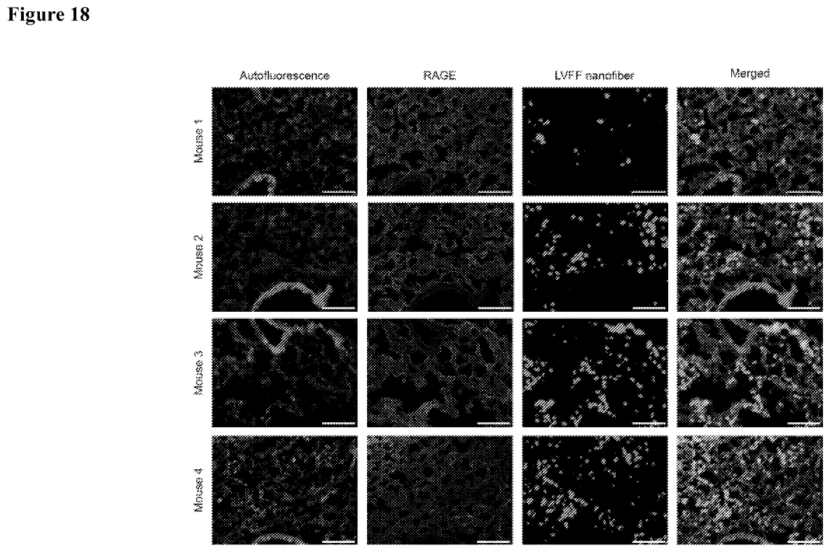

To demonstrate selective targeting of the “LVFF” (SEQ ID NO: 1) nanofiber to RAGE, immunofluorescence analysis was performed of lung tissue from nanofiber-treated hypoxic mice, which was stained for RAGE. Conventional fluorescence microscopy demonstrated colocalization of the “LVFF” (SEQ ID NO: 1) nanofiber to RAGE (FIG. 18), supporting the targeting mechanism of our nanomaterial.

### Example 15: Proportion of Targeted Epitope Incorporated in LVFF Nanofiber Affects Localization

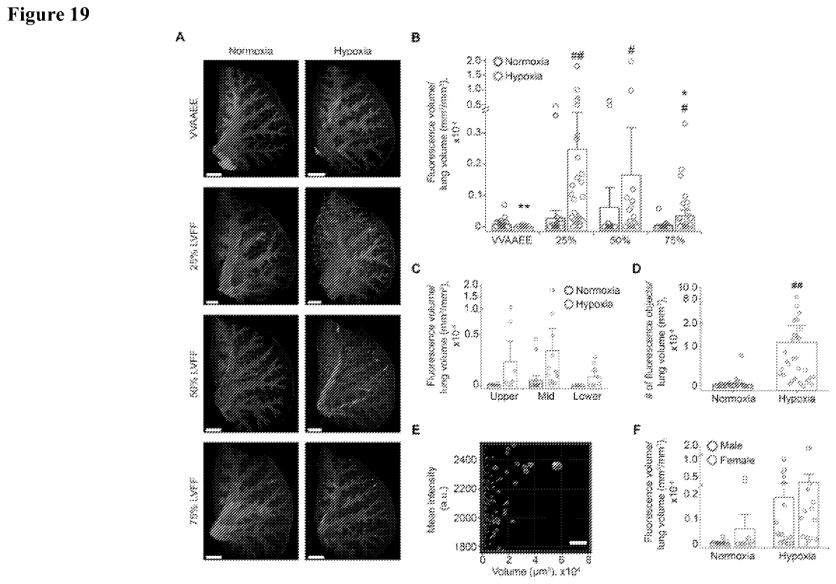

The “LVFF” (SEQ ID NO: 1) nanofiber was chosen for further in vivo analysis given its greater localization to the diseased lung compared to other targeted nanofibers. As prior evidence supports the important role of co-assembly on nanofiber structure and morphology, we sought to determine whether it also affects lung localization following intravascular delivery. Normoxic and hypoxic mice were injected with the “LVFF” (SEQ ID NO: 1) nanofiber at 25%, 50%, and 75% molar ratios of the targeting PA, and nanofiber accumulation within the lung was quantified using 3D LSFM (FIG. 19A). The 100% ratio was not investigated because it did not form fibers (FIG. 14C). Interestingly, “LVFF” (SEQ ID NO: 1) nanofibers containing less targeting epitope had greater localization to hypoxic lungs. The 25 mole % “LVFF” (SEQ ID NO: 1) nanofiber had the highest volumes of fluorescence per lung volume with an increased trend compared to the 50 mole % “LVFF” (SEQ ID NO: 1) nanofiber, and a significant increase compared to the 75 mole % “LVFF” (SEQ ID NO: 1) nanofiber (FIG. 19B). This inverse relationship between binding epitope concentration and lung targeting closely imitates patterns observed in fiber formation, in which “LVFF” (SEQ ID NO: 1) PA co-assembly ratios with 25% and 50% molar concentrations formed strong fibers, while the 75% and 100% co-assembly molar ratios made very poor, if any, fibers (FIG. 14C). In addition, it is possible that higher densities of the targeting epitope sterically hinder binding. Regardless, these data suggest that nanofiber co-assembly affects in vivo bioactivity via its impact on nanostructure formation.

Compared to the non-targeted VVAAEE (SEQ ID NO: 4) nanofiber, the 25 mole % “LVFF” (SEQ ID NO: 1) nanofiber had 480 times more fluorescence in diseased lungs (5.2×10⁻⁸±3.3×10⁻⁸ mm³/mm³ vs. 2.5×10⁻⁵±7.9×10⁻⁶ mm³/mm³, respectively; FIG. 19B). This striking difference further highlights the efficacy of the “LVFF” (SEQ ID NO: 1) nanofiber to localize to the lung. Importantly, at all co-assembly ratios, “LVFF” (SEQ ID NO: 1) nanofibers had significantly more localization to the diseased lung compared to normal lungs (FIG. 19B). Specifically, the 25 mole % “LVFF” (SEQ ID NO: 1) nanofiber demonstrated a 9-fold increase in localization in hypoxic versus normoxic mice.

Biodistribution analysis of the 25 mole % “LVFF” (SEQ ID NO: 1) nanofiber within hypoxic lungs revealed an even distribution throughout upper, middle, and lower lung regions (FIG. 19C). Similarly, an epifluorescence video showing a 3D reconstruction of a hypoxic mouse lung injected with the 25 mole % “LVFF” (SEQ ID NO: 1) nanofiber demonstrated uniform distribution. Since nanoparticle aggregation influences cellular uptake and binding avidity,^{[146]} the number of discrete fluorescent objects identified per lung volume (mm³) was measured to determine if high fluorescence volumes reflected large aggregates or well-dispersed fibers. Quantification revealed a significant increase in the number of distinct fluorescent objects in hypoxic versus normoxic mice (FIG. 19D), thus supporting uniform nanofiber distribution. Mean 25 mole % “LVFF” (SEQ ID NO: 1) nanofiber fluorescence intensity also closely corresponded to fluorescence volume measurements (FIG. 19E). There was no difference in fluorescence levels between male and female mice in either normoxic or hypoxic conditions (FIG. 19F).

### Example 16: Chemical and Physical Structure of LVFF Nanofiber Influences Lung Localization

The charge, physical structure, and stability of the 25 mole % “LVFF” (SEQ ID NO: 1) nanofiber was characterized to better understand its affinity to the lung in the setting of hypoxia-induced pulmonary hypertension. The “LVFF” (SEQ ID NO: 1) epitope consists of a hydrophobic patch abutted by two negatively charged residues at the C-terminus of the peptide sequence. With a net charge of −4 at neutral pH, the “LVFF” (SEQ ID NO: 1) PA was one of the most net negatively charged PAs that was tested (Table 7). This chemical property is likely important for nanofiber localization as RAGE is believed to be a pattern recognition receptor for negative charge and hydrophobicity.^{[131]} Several studies show that negatively charged residues are needed for RAGE binding.^{[131,147,148]} Although the exact binding mechanism is not completely understood, most RAGE ligands display a negative charge in neutral pH environments, further emphasizing the importance of charge in binding. Moreover, negatively charged residues are beneficial for nanoparticles in general. Some evidence suggests that negative charge may improve nanoparticle residence time in physiological environments,^{[149]} while positively charged particles have a propensity to be sequestered by macrophages in the lung.^{[105]} Evidence of cytotoxicity associated with positively charged nanomaterials was previously found,^{[150]} which is consistent with the literature.[^{149]}

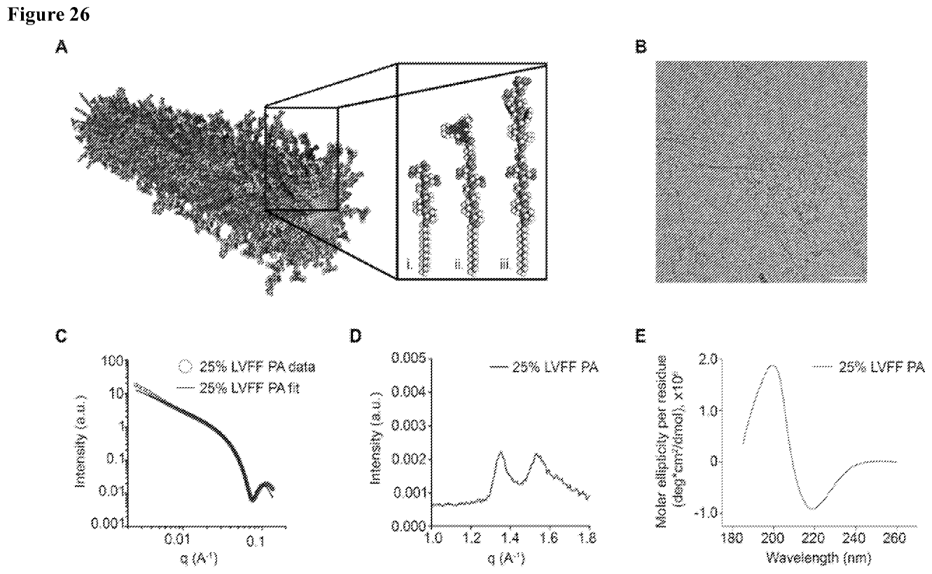

Co-assembled 25 mole % “LVFF” (SEQ ID NO: 1) PA nanofibers (FIG. 26A) were visualized in 10% fetal bovine serum (FBS) with cryogenic TEM (FIG. 26B). Fiber formation was unaffected by the presence of serum proteins, supporting nanofiber stability following intravascular delivery. Nanofiber structure was assessed using small-angle X-ray scattering (SAXS, FIG. 26C) and wide-angle X-ray scattering (WAXS, FIG. 26D). SAXS data were fitted to a polydisperse core-shell cylinder model to determine the average nanofiber diameter, which measured 9.7 nm (FIG. 26C). WAXS analysis showed a peak intensity at q=1.34 A⁻¹, consistent with the spacing in β-sheet structure (FIG. 26D). The second WAXS peak at q=1.55 A⁻¹ likely reflected packing of fibers (FIG. 26D). It is well-recognized that peptides with β-sheet structure have a propensity to self-assemble into multi-dimensional fibrils when immersed in aqueous solution.^{[151]} Similarly, circular dichroism spectroscopy analysis found a negative peak near 220 nm, further supporting a β-sheet structure (FIG. 26E).^{[152]}

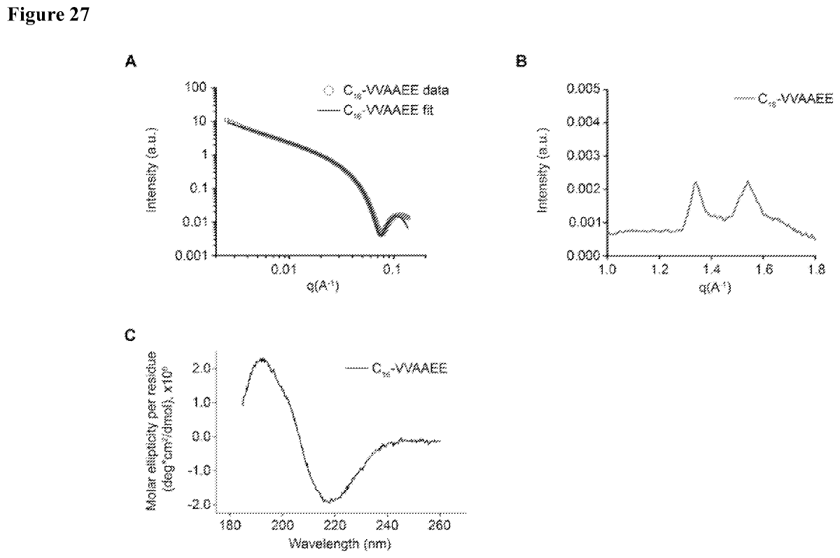

The non-targeted VVAAEE (SEQ ID NO: 4) nanofiber demonstrated β-sheet character by WAXS analysis and was found to have a similar diameter (9.2 nm) when the SAXS pattern was fitted to a polydisperse core-shell cylinder model (FIG. 27A-B). It also showed β-sheet structure at physiological temperature (37° C., FIG. 27C).

### Example 17: LVFF Nanofibers Remain Localized to the Lung for 24 Hours

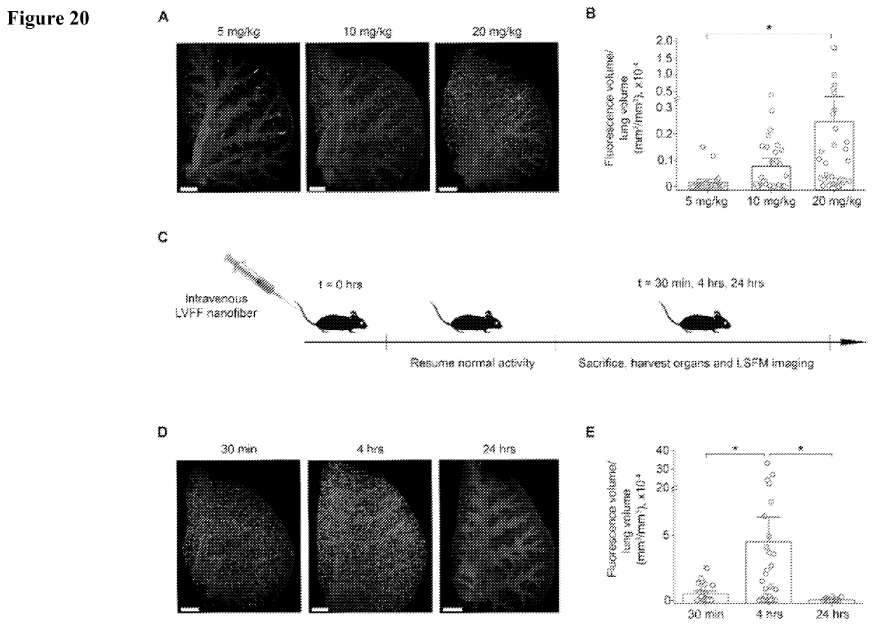

To determine the optimal dose of the 25 mole % “LVFF” (SEQ ID NO: 1) nanofiber, hypoxic mice were given a single intravenous dose of 5, 10, or 20 mg/kg, respectively (FIG. 20A). A dose-dependent increase in lung localization following intravenous delivery was observed, with significantly greater levels in mice injected with the highest dose (FIG. 20B). Even at the lowest dose, the targeted nanofiber had 29-fold more fluorescence volume per lung volume (1.5×10⁻⁶±7.0×10⁻⁷ mm³/mm³) compared to the non-targeted VVAAEE (SEQ ID NO: 4) nanofiber (5.2×10⁻⁸±3.3×10⁻⁸ mm³/mm³) when delivered at 4 times this dose (20 mg/kg).

Mice were sacrificed at 30 minutes, 4 hours and 24 hours post injection to determine duration of localization (FIG. 20C). The 25 mole % “LVFF” (SEQ ID NO: 1) nanofiber (20 mg/kg) began to localize to the lung as early as 30 minutes after injection (FIG. 20D), evidenced by a significant increase in fluorescence levels compared to the non-targeted nanofiber (p=0.008). By 4 hours, fluorescence increased 9-fold compared to levels observed at 30 minutes (FIG. 20E). The targeted nanofiber was retained in the lung for up to 24 hours after injection, at which point 99% had been cleared from the tissue (FIG. 20E).

### Example 18: Minimal LVFF Nanofiber Off-Target Localization

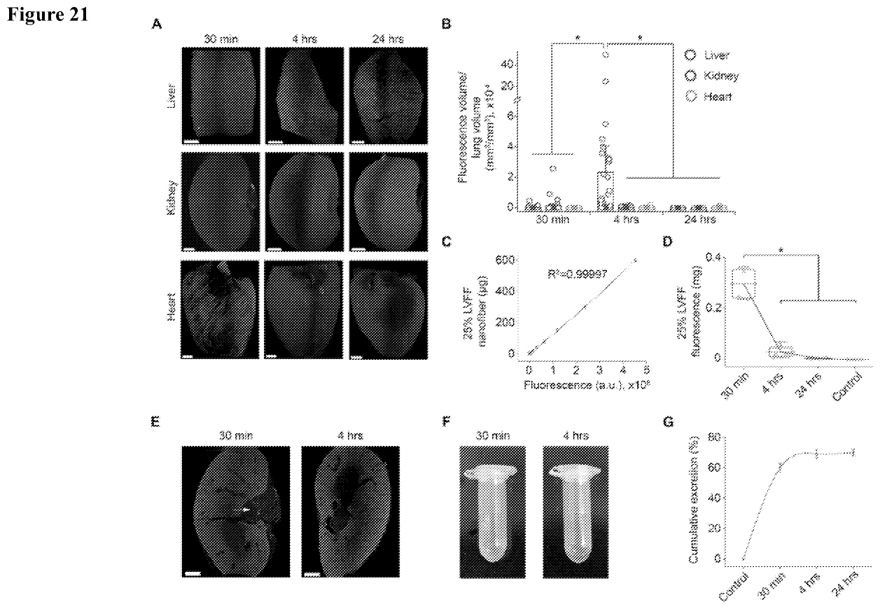

To confirm 25% LVFF nanofiber lung specificity, off-target localization to the liver, kidney, and heart in hypoxic mice was examined at 30 minutes, 4 hours, and 24 hours after injection (FIG. 21A). Similar to prior analysis, quantification of nanofiber distribution was measured as fluorescence volume per tissue volume (mm³/mm³) using 3D LSFM. Overall, the “LVFF” (SEQ ID NO: 1) nanofiber demonstrated greater specificity for the lung at all evaluated time points, with a consistently significant difference in fluorescence volume per tissue volume compared to the kidney and heart (p<0.001, respectively, FIGS. 20E and 21B). Nanofiber localization was significantly higher in the lung versus the liver at 30 minutes and 24 hours (p<0.001, respectively), with a trend towards increased localization at 4 hours (p=0.43). This is likely due to a rise in nanofiber accumulation within the liver 4 hours after injection (FIGS. 20E and 21B). This spike in nanofiber localization to the liver may be associated with the onset of nanoparticle metabolism by hepatic enzymes, particularly since fluorescence levels remained minimal in the heart and kidney during this time point. However, even at 4 hours, fluorescence in the lung was still nearly 2-fold higher than the liver (FIGS. 20E and 21B). At 24 hours, fluorescence was markedly reduced across all organs suggesting nanofiber metabolism and excretion from the body.

Urine samples were collected at 30 minutes, 4 hours, and 24 hours post injection to evaluate renal excretion. Fluorescence intensity of standardized amounts of the 25 mole % “LVFF” (SEQ ID NO: 1) nanofiber (μg), which were dissolved in 50 μL of Hank's Balanced Salt Solution (HBSS), were measured to determine a dose-response curve (FIG. 21C). This revealed a linear relationship between the two variables (R²>0.99), and was used to calculate the amount of nanofiber fluorescence in the urine of the injected mice. Given that mice were awoken immediately after injection, voids that occurred in the time elapsed between injection and time of sacrifice were not captured. Non-injected hypoxic mice served as controls. At 30 minutes, the amount of fluorescence excreted in the urine was significantly higher in treated hypoxic mice compared to non-injected hypoxic controls (FIG. 21D). This corresponds to LSFM findings, which showed an accumulation of nanofibers within the collecting duct of the kidney exclusively at 30 minutes (FIG. 21E). Not surprisingly, urine samples at 30 minutes post injection were bright pink on gross examination (FIG. 21F), closely mimicking the color of the fluorescently labeled nanofiber stock solution. At 4 hours post injection, urine fluorescence measurements demonstrated significantly less fluorescence intensity. By 24 hours, urine fluorescence was similar to levels seen in non-injected hypoxic mice.

The cumulative percentage of fluorescent renal excretion with respect to the total fluorescence injected with the fluorophore-labeled nanofiber was calculated (FIG. 21G). At 30 minutes, fluorescence in the urine of the treated hypoxic mice represented 60% of fluorescence of the injected nanofiber stock solution (i.e., 0.29 mg in the urine vs. 0.49 mg injected). We theorize that rapid renal excretion of excess, unbound nanofiber is likely due to the nanomaterial's small size. Previous evidence shows that molecules smaller than 10 nm are susceptible to rapid renal clearance and nanoparticles less than 5.5 nm are entirely eliminated in the urine.^{[112]} After 24 hours, cumulative excretion plateaued leaving 30% of the fluorescence unaccounted for. We suspect that some may have been excreted in the time interval between injection and organ harvesting, as urine was not collected then and thus, not included in this analysis.

Pulmonary diseases, such as pulmonary hypertension, are challenging to treat because most medications are administered systemically in order to reach the diseased pulmonary system, thus increasing the risk for off-target toxicity and rapid proteolytic destruction. Non-specific biodistribution limits safety and efficacy of current Food and Drug Administration (FDA)-approved pharmacotherapies for pulmonary hypertension. We theorized that targeted drug delivery to the pulmonary vasculature via a PA nanofiber could offer a promising approach to mitigate these drug-related off-target side effects. In this study, we demonstrated the successful design and synthesis of an injectable, RAGE-targeted PA nanofiber delivery platform that effectively localizes to the diseased lung for up to 24 hours in a preclinical model of hypoxia-induced pulmonary hypertension.

RAGE is expressed in pulmonary artery endothelial cells and SMCs within the pulmonary vasculature.^{[153]} Early in disease progression, patients with pulmonary arterial hypertension develop a 6-fold increase in RAGE expression in pulmonary artery SMCs, while upregulation in endothelial cells presents in later stages of disease.^{[154]} Accumulating evidence reveals that RAGE is critical for vascular remodeling processes that are predominantly responsible for poor disease prognosis. Ligand binding induces activation of RAGE to stimulate vasoconstriction, cellular hyperproliferation, and pro-inflammatory processes in the pulmonary vasculature.^{[155,156]} Accordingly, RAGE-mediated cellular responses are predicated on the type of ligand, the binding mechanism to RAGE including changes in structural conformation, and the state of the surrounding extracellular environment. As ligands accumulate at the site of disease, their increased expression stimulates the upregulation of RAGE in a positive feedback system to further promote activation of molecular pathogenic pathways. Once bound, ligands are not modified or degraded, thus persistently worsening disease. Evidence suggests that inhibition of RAGE expression or RAGE ligand binding prevents pulmonary hypertension progression^{[154]} via its modifications to vascular remodeling, which is poorly addressed by available pharmacotherapies. Meloche et al. demonstrated that RAGE inhibition improved pulmonary perfusion and mitigated vascular remodeling in experimental rat models of pulmonary hypertension and reversed pulmonary hypertension cellular phenotype in human pulmonary artery SMCs.^{[154]} Importantly, downstream proteins in RAGE signaling pathways cannot be easily targeted as most of these molecules are ubiquitously expressed throughout the body.

From a clinical perspective, targeting of RAGE for therapeutic gain is promising because it can be applied to a broad spectrum of disease. RAGE is involved in many cardiovascular and inflammatory diseases, including diabetic vasculopathy, atherosclerosis,^{[157]} arthritis,^{[158]} and transplantation.^{[159]} Such a diverse range of clinical pathology presents a unique opportunity for RAGE-targeted nanofibers to be evaluated for efficacy and versatility in numerous disease models. Moreover, since RAGE has an abundance of ligands, there are more binding sequences available to investigate which can be used to develop new therapies for pulmonary hypertension patients. The binding domain of RAGE is highly conserved across many species,^{[160]} enhancing the likelihood of successful translation of a RAGE-targeting therapeutic agent into the clinical arena.

Interestingly, out of the RAGE-targeted nanofibers that were tested in this study, each had a targeted epitope derived from a different RAGE ligand. The “LVFF” (SEQ ID NO: 1) nanofiber, which demonstrated superior efficacy for lung localization in our chronic hypoxia-induced pulmonary hypertension mouse model was selected from the Aβ ligand. Unlike S100 proteins^{[161]} and HMGB1,^{[121]} which were used to construct the RAGE-targeted “AMV” (SEQ ID NO: 9) and “KGVV” (SEQ ID NO: 3) nanofibers, respectively, Aβ is not known to be involved in pulmonary hypertension. Thus, we theorize that an abundance of S100 and HMGB1 ligands in the diseased lungs competitively inhibited “AMV” (SEQ ID NO: 9) and “KGVV” (SEQ ID NO: 3) nanofibers from targeting to RAGE due to similar binding mechanisms. While the exact molecular mechanism of RAGE ligand binding is not completely understood, charge and structural configuration are theorized to be important for binding affinity to RAGE. Thus, a slight variation in binding by Aβ may account for the improved targeting ability of the “LVFF” (SEQ ID NO: 1) nanofiber.

The application of a biocompatible RAGE-targeted nanomaterial that can inhibit RAGE-mediated signaling may reduce vascular remodeling and improve pulmonary hypertension, even in the absence of a therapeutic agent. Self-assembled PA nanofibers that can be easily designed to recognize select proteins over a large surface area are ideal for targeted intervention in pulmonary-specific conditions.^{[162-165]} Their small size allows for longer circulation in vivo, and is advantageous for targeting of the lung as the pulmonary circulation is known to retain small nanoparticles (7 μm or larger).^{[166]} This mechanism is exploited in pulmonary hypertension, as endothelial cell dysfunction is highly involved in disease pathophysiology, and contributes to vascular fenestration.^{[155]} Porous endothelial cell basement membranes that physically entrap small molecules may explain why the non-targeted nanofiber and the other ACE- and RAGE-targeted nanofibers still exhibit some fluorescence signal in the lung. The drastic difference in lung levels between these nanofibers and the RAGE-targeted “LVFF” (SEQ ID NO: 1) nanofiber supports that localization of the “LVFF” (SEQ ID NO: 1) nanofiber was mediated by effective targeting to RAGE.

Due to cumbersome and time-consuming tissue preparation, we were unable to evaluate “LVFF” (SEQ ID NO: 1) nanofiber and RAGE colocalization on LSFM to quantify targeting efficacy. Furthermore, an in vitro analysis to quantitatively characterize nanofiber binding affinity was not performed as our laboratory has found from prior work that in vitro binding does not reflect in vivo binding in which many variables come into play that simply cannot be recapitulated in the in vitro environment. In addition, we have observed that the nanofiber exhibits nonspecific adhesion to plastic surfaces, further introducing challenges with the in vitro assays that limits its application. While future mechanistic studies are necessary to quantify binding constants and determine which cell types the nanofiber is binding to, we are reassured by our in vivo findings that demonstrated similar distribution patterns of RAGE in the lung compared to LSFM images of “LVFF” (SEQ ID NO: 1) nanofiber lung localization following intravascular delivery. Similarly, 2D conventional fluorescence microscopy also showed colocalization of RAGE and “LVFF” (SEQ ID NO: 1) nanofibers in hypoxic mice, although we had insufficient data for quantification analysis. Together, these findings support successful targeting of RAGE in pulmonary hypertension. Safety and off-target toxicity were also not assessed in this study. Although we did not collect blood samples to evaluate hepatic and renal function, previous analysis by our laboratories found no evidence of renal or hepatic toxicity following intravenous PA nanofiber delivery in vivo.^{[115]} Therefore, we do not anticipate off-target toxicity in animals treated with the RAGE-targeted “LVFF” (SEQ ID NO: 1) nanofiber. Anecdotally, mice behaved normally following injections with no evidence of severe adverse effects within 24 hours after treatment.

In summary, our results demonstrate that intravascular RAGE-targeted nanofibers localize to diseased pulmonary tissue in an experimental model of hypoxia-induced pulmonary hypertension. Future studies will need to evaluate the effect of “LVFF” (SEQ ID NO: 1) nanofiber targeting on histological and cardiopulmonary markers of pulmonary hypertension pathophysiology to determine safety and potential therapeutic efficacy. This delivery platform may serve as the foundation for the development of a nanomaterials-based therapy to effectively treat pulmonary hypertension through specific targeting of therapeutic agents to the lung, avoiding systemic side effects.

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### BIBLIOGRAPHY

1. Wolf L, Herr C, Niederstrasser J, Beisswenger C, Bals R. Receptor for advanced glycation endproducts (RAGE) maintains pulmonary structure and regulates the response to cigarette smoke. PLoS One. 2017; 12(7):e0180092.
2. Lee S, Piao C, Kim G, Kim J Y, Choi E, Lee M. Production and application of HMGB1 derived recombinant RAGE-antagonist peptide for anti-inflammatory therapy in acute lung injury. Eur J Pharm Sci. 2018; 114:275-284.
3. Blondonnet R, Audard J, Belville C, et al. RAGE inhibition reduces acute lung injury in mice. Sci Rep. 2017; 7(1):7208.
4. Yilin Z, Yandong N, Faguang J. Role of angiotensin-converting enzyme (ACE) and ACE2 in a rat model of smoke inhalation induced acute respiratory distress syndrome. Burns. 2015; 41(7):1468-1477.
5. Li W, Qiu X, Wang J, et al. The therapeutic efficacy of glutamine for rats with smoking inhalation injury. Int Immunopharmacol. 2013; 16(2):248-253.
6. Katahira J, Murakami K, Schmalstieg F C, et al. Role of anti-L-selectin antibody in burn and smoke inhalation injury in sheep. Am J Physiol Lung Cell Mol Physiol. 2002; 283(5):L1043-1050.
7. Dunn J L M, Kartchner L B, Stepp W H, et al. Blocking CXCL1-dependent neutrophil recruitment prevents immune damage and reduces pulmonary bacterial infection after inhalation injury. Am J Physiol Lung Cell Mol Physiol. 2018; 314(5):L822-L834.
8. Ding H, Lv Q, Wu S, et al. Intratracheal Instillation of Perfluorohexane Modulates the Pulmonary Immune Microenvironment by Attenuating Early Inflammatory Factors in Patients With Smoke Inhalation Injury: A Randomized Controlled Clinical Trial. J Burn Care Res. 2017; 38(4):251-259.
9. Wang X, Zhang J, Li X, et al. Sustained improvement of gas exchange and lung mechanics by vaporized perfluorocarbon inhalation in piglet acute lung injury model. Clin Respir J. 2014; 8(2):160-166.
10. Saunders F D, Westphal M, Enkhbaatar P, et al. Molecular biological effects of selective neuronal nitric oxide synthase inhibition in ovine lung injury. Am J Physiol Lung Cell Mol Physiol. 2010; 298(3):L427-436.
11. Soejima K, Traber L D, Schmalstieg F C, et al. Role of nitric oxide in vascular permeability after combined burns and smoke inhalation injury. Am J Respir Crit Care Med. 2001; 163(3 Pt 1):745-752.
12. Guo G H, Sun W. [Goal-targeted therapy of inhalation injury with intratracheal delivery of drugs]. Zhonghua Shao Shang Za Zhi. 2018; 34(7):445-449.
13. Pandareesh M D, Anand T. Attenuation of smoke induced neuronal and physiological changes by bacoside rich extract in Wistar rats via down regulation of HO-1 and iNOS. Neurotoxicology. 2014; 40:33-42.
14. Lange M, Connelly R, Traber D L, et al. Combined neuronal and inducible nitric oxide synthase inhibition in ovine acute lung injury. Crit Care Med. 2009; 37(1):223-229.
15. Lange M, Szabo C, Enkhbaatar P, et al. Beneficial pulmonary effects of a metalloporphyrinic peroxynitrite decomposition catalyst in burn and smoke inhalation injury. Am J Physiol Lung Cell Mol Physiol. 2011; 300(2):L167-175.
16. Hamahata A, Enkhbaatar P, Lange M, et al. Administration of a peroxynitrite decomposition catalyst into the bronchial artery attenuates pulmonary dysfunction after smoke inhalation and burn injury in sheep. Shock. 2012; 38(5):543-548.
17. I to H, Malgerud E, Asmussen S, Lopez E, Salzman A L, Enkhbaatar P. R-100 improves pulmonary function and systemic fluid balance in sheep with combined smoke-inhalation injury and Pseudomonas aeruginosa sepsis. J Transl Med. 2017; 15(1):266.
18. Powell C R, Dillon K M, Matson J B. A review of hydrogen sulfide (H₂S) donors: Chemistry and potential therapeutic applications. Biochem Pharmacol. 2018; 149:110-123.
19. Li T, Zhao B, Wang C, et al. Regulatory effects of hydrogen sulfide on IL-6, IL-8 and IL-10 levels in the plasma and pulmonary tissue of rats with acute lung injury. Exp Biol Med (Maywood). 2008; 233(9):1081-1087.
20. Han Z H, Jiang Y I, Duan Y Y, Wang X Y, Huang Y, Fang T Z. Protective effects of hydrogen sulfide inhalation on oxidative stress in rats with cotton smoke inhalation-induced lung injury. Exp Ther Med. 2015; 10(1):164-168.
21. Carvalho F O, Silva E R, Nunes P S, et al. Effects of the solid lipid nanoparticle of carvacrol on rodents with lung injury from smoke inhalation. Naunyn Schmiedebergs Arch Pharmacol. 2019.
22. Morgan C E, Dombrowski A W, Rubert Perez C M, et al. Tissue-Factor Targeted Peptide Amphiphile Nanofibers as an Injectable Therapy To Control Hemorrhage. ACS Nano. 2016; 10(1):899-909.
23. Bahnson E S, Kassam H A, Moyer T J, et al. Targeted Nitric Oxide Delivery by Supramolecular Nanofibers for the Prevention of Restenosis After Arterial Injury. Antioxid Redox Signal. 2016; 24(8):401-418.
24. Shue E H, Schecter S C, Gong W, et al. Antenatal maternally-administered phosphodiesterase type 5 inhibitors normalize eNOS expression in the fetal lamb model of congenital diaphragmatic hernia. J Pediatr Surg. 2014; 49(1):39-45; discussion 45.
25. Bialkowski A, Moenkemeyer F, Patel N. Intravenous sildenafil in the management of pulmonary hypertension associated with congenital diaphragmatic hernia. Eur J Pediatr Surg. 2015; 25(2):171-176.
26. Noon S, Friedlich P, Wong P, Garingo A, Seri I. Cardiovascular effects of sildenafil in neonates and infants with congenital diaphragmatic hernia and pulmonary hypertension. Neonatology. 2007; 91(2):92-100.
27. Barnett C F, Machado R F. Sildenafil in the treatment of pulmonary hypertension. Vasc Health Risk Manag. 2006; 2(4):411-422.
28. Ghasemian E, Vatanara A, Rouholamini Najafabadi A, Rouini M R, Gilani K, Darabi M. Preparation, characterization and optimization of sildenafil citrate loaded PLGA nanoparticles by statistical factorial design. Daru. 2013; 21(1):68.
29. Ghofrani H A, Morrell N W, Hoeper M M, et al. Imatinib in pulmonary arterial hypertension patients with inadequate response to established therapy. Am J Respir Crit Care Med. 2010; 182(9):1171-1177.
30. Marslin G, Revina A M, Khandelwal V K, et al. Delivery as nanoparticles reduces imatinib mesylate-induced cardiotoxicity and improves anticancer activity. Int J Nanomedicine. 2015; 10:3163-3170.
31. Gosemann J H, Friedmacher F, Hofmann A, et al. Prenatal treatment with rosiglitazone attenuates vascular remodeling and pulmonary monocyte influx in experimental congenital diaphragmatic hernia. PLoS One. 2018; 13(11):e0206975.
32. Liu Y, Tian X Y, Huang Y, Wang N. Rosiglitazone Attenuated Endothelin-1-Induced Vasoconstriction of Pulmonary Arteries in the Rat Model of Pulmonary Arterial Hypertension via Differential Regulation of ET-1 Receptors. PPAR Res. 2014; 2014:374075.
33. Rashid J, Alobaida A, Al-Hilal T A, et al. Repurposing rosiglitazone, a PPAR-gamma agonist and oral antidiabetic, as an inhaled formulation, for the treatment of PAH. J Control Release. 2018; 280:113-123.
34. Nishimura T, Faul J L, Berry G J, et al. Simvastatin attenuates smooth muscle neointimal proliferation and pulmonary hypertension in rats. Am J Respir Crit Care Med. 2002; 166(10):1403-1408.
35. Nishimura T, Vaszar L T, Faul J L, et al. Simvastatin rescues rats from fatal pulmonary hypertension by inducing apoptosis of neointimal smooth muscle cells. Circulation. 2003; 108(13):1640-1645.
36. Ichimura K, Matoba T, Koga J I, et al. Nanoparticle-Mediated Targeting of Pitavastatin to Small Pulmonary Arteries and Leukocytes by Intravenous Administration Attenuates the Progression of Monocrotaline-Induced Established Pulmonary Arterial Hypertension in Rats. Int Heart 1 2018; 59(6):1432-1444.
37. Chen L, Nakano K, Kimura S, et al. Nanoparticle-mediated delivery of pitavastatin into lungs ameliorates the development and induces regression of monocrotaline-induced pulmonary artery hypertension. Hypertension. 2011; 57(2):343-350.
38. Morgan C E, Dombrowski A W, Rubert Perez C M, et al. Tissue-Factor Targeted Peptide Amphiphile Nanofibers as an Injectable Therapy To Control Hemorrhage. ACS Nano. 2016; 10(1):899-909.
39. O'Callaghan D S, Savale L, Jais X, et al. Evidence for the use of combination targeted therapeutic approaches for the management of pulmonary arterial hypertension. Respir Med. 2010; 104 Suppl 1:S74-80.
40. Humbert M, Guignabert C, Bonnet S, et al. Pathology and pathobiology of pulmonary hypertension: state of the art and research perspectives. The European respiratory journal. 2018.
41. Dai Z, Li M, Wharton J, Zhu M M, Zhao Y Y. Prolyl-4 Hydroxylase 2 (PHD2) Deficiency in Endothelial Cells and Hematopoietic Cells Induces Obliterative Vascular Remodeling and Severe Pulmonary Arterial Hypertension in Mice and Humans Through Hypoxia-Inducible Factor-2alpha. Circulation. 2016; 133(24):2447-2458.
43. Hartgerink J D, Beniash E, Stupp S I. Self-assembly and mineralization of peptide-amphiphilen nanofibers. Science. 2001; 294(5547):1684-1688.
44. Bahnson E S, Kassam H A, Moyer T J, et al. Targeted Nitric Oxide Delivery by Supramolecular Nanofibers for the Prevention of Restenosis After Arterial Injury. Antioxid Redox Signal. 2016; 24(8):401-418.
45. Morgan C E, Dombrowski A W, Rubert Perez C M, et al. Tissue-Factor Targeted Peptide Amphiphile Nanofibers as an Injectable Therapy To Control Hemorrhage. ACS Nano. 2016; 10(1):899-909.
46. Moyer T J, Kassam H A, Bahnson E S, et al. Shape-Dependent Targeting of Injured Blood Vessels by Peptide Amphiphile Supramolecular Nanostructures. Small. 2015; 11(23):2750-2755.
47. Nakamura K, Sakaguchi M, Matsubara H, et al. Crucial role of RAGE in inappropriate increase of smooth muscle cells from patients with pulmonary arterial hypertension. PLoS One. 2018; 13(9):e0203046.
48. Jia D, He Y, Zhu Q, et al. RAGE-mediated extracellular matrix proteins accumulation exacerbates HySu-induced pulmonary hypertension. Cardiovasc Res. 2017; 113(6):586-597.
49. Meloche J, Courchesne A, Barrier M, et al. Critical role for the advanced glycation end-products receptor in pulmonary arterial hypertension etiology. J Am Heart Assoc. 2013; 2(1):e005157.
50. Lettieri C J, Nathan S D, Barnett S D, Ahmad S, Shorr A F. Prevalence and outcomes of pulmonary arterial hypertension in advanced idiopathic pulmonary fibrosis. Chest. 2006; 129(3):746-752.
51. de Jesus Perez V A. Molecular pathogenesis and current pathology of pulmonary hypertension. Heart Fail Rev. 2016; 21(3):239-257.
52. Dos Santos Fernandes C J C, Humbert M, Souza R. Challenging the concept of adding more drugs in pulmonary arterial hypertension. The European respiratory journal. 2017; 50(3).
53. George M G, Schieb L J, Ayala C, Talwalkar A, Levant S. Pulmonary hypertension surveillance: United States, 2001 to 2010. Chest. 2014; 146(2):476-495.
54. Segura-Ibarra V, Wu S, Hassan N, et al. Nanotherapeutics for Treatment of Pulmonary Arterial Hypertension. Front Physiol. 2018; 9:890.
55. Voelkel N F, Tuder R M. Hypoxia-induced pulmonary vascular remodeling: a model for what human disease? J Clin Invest. 2000; 106(6):733-738.
56. Shah M, Phillips M R, Quintana M, Stupp G, McLean S E. Echocardiography allows for analysis of pulmonary arterial flow in mice with congenital diaphragmatic hernia. J Surg Res. 2018; 221:35-42.
57. S. S. Kadri, A. C. Miller, S. Hohmann, S. Bonne, C. Nielsen, C. Wells, C. Gruver, S. A. Quraishi, J. Sun, R. Cai, P. E. Morris, B. D. Freeman, J. H. Holmes, B. A. Cairns, A. F. Suffredini, U.S.C. Illness, I. Injury Trials Group: Smoke Inhalation-associated Acute Lung Injury, Risk Factors for In-Hospital Mortality in Smoke Inhalation-Associated Acute Lung Injury: Data From 68 United States Hospitals, Chest 150(6) (2016) 1260-1268.
58. P. Enkhbaatar, B. A. Pruitt, Jr., O. Suman, R. Mlcak, S. E. Wolf, H. Sakurai, D. N. Herndon, Pathophysiology, research challenges, and clinical management of smoke inhalation injury, Lancet 388(10052) (2016) 1437-1446.
59. S. W. Jones, F. N. Williams, B. A. Cairns, R. Cartotto, Inhalation Injury: Pathophysiology, Diagnosis, and Treatment, Clin Plast Surg 44(3) (2017) 505-511.
60. M. Doroudian, R. MacLoughlin, F. Poynton, A. Prina-Mello, S. C. Donnelly, Nanotechnology based therapeutics for lung disease, Thorax 74(10) (2019) 965-976.
61. M. He, J. Zhu, N. Yu, H. Kong, X. Zeng, W. Xie, H. Xu, The Superior Antitumor Effect of Self-Assembled Paclitaxel Nanofilaments for Lung Cancer Cells, Curr Drug Deliv 16(2) (2019) 171-178.
62. R. Iyer, C. C. Hsia, K. T. Nguyen, Nano-Therapeutics for the Lung: State-of-the-Art and Future Perspectives, Curr Pharm Des 21(36) (2015) 5233-44.
63. C. Schleh, B. Rothen-Rutishauser, W. G. Kreyling, The influence of pulmonary surfactant on nanoparticulate drug delivery systems, Eur J Pharm Biopharm 77(3) (2011) 350-2.
64. J. D. Hartgerink, E. Beniash, S. I. Stupp, Self-assembly and mineralization of peptide-amphiphile nanofibers, Science 294(5547) (2001) 1684-8.
65. C. E. Morgan, A. W. Dombrowski, C. M. Rubert Perez, E. S. Bahnson, N. D. Tsihlis, W. Jiang, Q. Jiang, J. M. Vercammen, V. S. Prakash, T. A. Pritts, S. I. Stupp, M. R. Kibbe, Tissue-Factor Targeted Peptide Amphiphile Nanofibers as an Injectable Therapy To Control Hemorrhage, ACS Nano 10(1) (2016) 899-909.
66. N. A. Mansukhani, E. B. Peters, M. M. So, M. S. Albaghdadi, Z. Wang, M. R. Karver, T. D. Clemons, J. P. Laux, N. D. Tsihlis, S. I. Stupp, M. R. Kibbe, Peptide Amphiphile Supramolecular Nanostructures as a Targeted Therapy for Atherosclerosis, Macromol Biosci 19(6) (2019) e1900066.
67. E. B. Peters, N. D. Tsihlis, M. R. Karver, S. M. Chin, B. Musetti, B. T. Ledford, E. M. Bahnson, S. I. Stupp, M. R. Kibbe, Atheroma Niche-Responsive Nanocarriers for Immunotherapeutic Delivery, Adv Healthc Mater 8(3) (2019) e1801545.
68. E. S. Bahnson, H. A. Kassam, T. J. Moyer, W. Jiang, C. E. Morgan, J. M. Vercammen, Q. Jiang, M. E. Flynn, S. I. Stupp, M. R. Kibbe, Targeted Nitric Oxide Delivery by Supramolecular Nanofibers for the Prevention of Restenosis After Arterial Injury, Antioxid Redox Signal 24(8) (2016) 401-18.
69. Z. Yilin, N. Yandong, J. Faguang, Role of angiotensin-converting enzyme (ACE) and ACE2 in a rat model of smoke inhalation induced acute respiratory distress syndrome, Burns 41(7) (2015) 1468-77.
70. N. Demling, C. Ehrhardt, M. Kasper, M. Laue, L. Knels, E. P. Rieber, Promotion of cell adherence and spreading: a novel function of RAGE, the highly selective differentiation marker of human alveolar epithelial type I cells, Cell Tissue Res 323(3) (2006) 475-88.
71. S. Lee, C. Piao, G. Kim, J. Y. Kim, E. Choi, M. Lee, Production and application of HMGB1 derived recombinant RAGE-antagonist peptide for anti-inflammatory therapy in acute lung injury, Eur J Pharm Sci 114 (2018) 275-284.
72. D. J. Toft, T. J. Moyer, S. M. Standley, Y. Ruff, A. Ugolkov, S. I. Stupp, V. L. Cryns, Coassembled cytotoxic and pegylated peptide amphiphiles form filamentous nanostructures with potent antitumor activity in models of breast cancer, ACS Nano 6(9) (2012) 7956-65.
73. J. Ilaysky, P. R. Jemian, Irena: tool suite for modeling and analysis of small-angle scattering, Journal of Applied Crystallography 42 (2009) 347-353.
74. A. I. Mercel, D. C. Gillis, K. Sun, B. R. Dandurand, J. M. Weiss, N. D. Tsihlis, R. Maile, M. R. Kibbe, A Comparative Study of a Pre-Clinical Survival Model of Smoke Inhalation Injury in Mice and Rats, Am J Physiol Lung Cell Mol Physiol (2020).
75. H. Wu, Y. Liu, M. Guo, J. Xie, X. Jiang, A virtual screening method for inhibitory peptides of Angiotensin I-converting enzyme, J Food Sci 79(9) (2014) C1635-42.
76. L. Z. Yalan Liu, Mingrong Guo, Hongxi Wu, Jingli Xie,nd Dongzhi Weil, Virtual screening for angiotensin I-converting enzyme inhibitory peptides from Phascolosoma esculenta, Bioresour. Bioprocess. 1(17) (2014).
77. X. C. Mingrong Guoa, Yanling Wua, Lujia Zhanga, Weixue Huangb, Ying Yuanc, Ming Fanga, Jingli Xiea, Dongzhi Wei, Angiotensin I-converting enzyme inhibitory peptides from Sipuncula (Phascolosoma esculenta): Purification, identification, molecular docking and antihypertensive effects on spontaneously hypertensive rats, Process Biochemistry 63 (2017) 84-95.
78. H. Ni, L. Li, G. Liu, S. Q. Hu, Inhibition mechanism and model of an angiotensin I-converting enzyme (ACE)-inhibitory hexapeptide from yeast (Saccharomyces cerevisiae), PLoS One 7(5) (2012) e37077.
79. C. A. Downs, N. M. Johnson, G. Tsaprailis, M. N. Helms, RAGE-induced changes in the proteome of alveolar epithelial cells, J Proteomics 177 (2018) 11-20.
80. E. Gospodarska, A. Kupniewska-Kozak, G. Goch, M. Dadlez, Binding studies of truncated variants of the Abeta peptide to the V-domain of the RAGE receptor reveal Abeta residues responsible for binding, Biochim Biophys Acta 1814(5) (2011) 592-609.
81. J. Xue, M. Manigrasso, M. Scalabrin, V. Rai, S. Reverdatto, D. S. Burz, D. Fabris, A. M. Schmidt, A. Shekhtman, Change in the Molecular Dimension of a RAGE-Ligand Complex Triggers RAGE Signaling, Structure 24(9) (2016) 1509-22.
82. H. Cui, A. G. Cheetham, E. T. Pashuck, S. I. Stupp, Amino acid sequence in constitutionally isomeric tetrapeptide amphiphiles dictates architecture of one-dimensional nanostructures, J Am Chem Soc 136(35) (2014) 12461-8.
83. V. I. Dodero, Z. B. Quirolo, M. A. Sequeira, Biomolecular studies by circular dichroism, Front Biosci (Landmark Ed) 16 (2011) 61-73.
84. R. A. Petros, J. M. DeSimone, Strategies in the design of nanoparticles for therapeutic applications, Nat Rev Drug Discov 9(8) (2010) 615-27.
85. S. Azarmi, W. H. Roa, R. Lobenberg, Targeted delivery of nanoparticles for the treatment of lung diseases, Adv Drug Deliv Rev 60(8) (2008) 863-75.
86. J. Nicholls, M. Peiris, Good ACE, bad ACE do battle in lung injury, SARS, Nat Med 11(8) (2005) 821-2.
87. Y. Imai, K. Kuba, S. Rao, Y. Huan, F. Guo, B. Guan, P. Yang, R. Sarao, T. Wada, H. Leong-Poi, M. A. Crackower, A. Fukamizu, C. C. Hui, L. Hein, S. Uhlig, A. S. Slutsky, C. Jiang, J. M. Penninger, Angiotensin-converting enzyme 2 protects from severe acute lung failure, Nature 436(7047) (2005) 112-6.
88. K. Kuba, Y. Imai, J. M. Penninger, Angiotensin-converting enzyme 2 in lung diseases, Curr Opin Pharmacol 6(3) (2006) 271-6.
89. A. J. Omlor, J. Nguyen, R. Bals, Q. T. Dinh, Nanotechnology in respiratory medicine, Respir Res 16 (2015) 64.
90. S. Hassan, G. Prakash, A. Ozturk, S. Saghazadeh, M. F. Sohail, J. Seo, M. Dockmeci, Y. S. Zhang, A. Khademhosseini, Evolution and Clinical Translation of Drug Delivery Nanomaterials, Nano Today 15 (2017) 91-106.
91. D. Saputra, J. H. Yoon, H. Park, Y. Heo, H. Yang, E. J. Lee, S. Lee, C. W. Song, K. Lee, Inhalation of carbon black nanoparticles aggravates pulmonary inflammation in mice, Toxicol Res 30(2) (2014) 83-90.
92. Y. Zhang, Y. Huang, S. Li, Polymeric micelles: nanocarriers for cancer-targeted drug delivery, AAPS PharmSciTech 15(4) (2014) 862-71.
93. Y. Akasaka, H. Ueda, K. Takayama, Y. Machida, T. Nagai, Preparation and evaluation of bovine serum albumin nanospheres coated with monoclonal antibodies, Drug Des Deliv 3(1) (1988) 85-97.
94. S. Muro, V. R. Muzykantov, Targeting of antioxidant and anti-thrombotic drugs to endothelial cell adhesion molecules, Curr Pharm Des 11(18) (2005) 2383-401.
95. F. O. Carvalho, E. R. Silva, P. S. Nunes, F. A. Felipe, K. P. P. Ramos, L. A. S. Ferreira, V. N. B. Lima, S. Shanmugam, A. S. Oliveira, S. S. Guterres, E. A. Camargo, T. V. Cravalho Olivera, R. L. C. de Albuquerque Junior, W. de Lucca Junior, L. J. Quintans-Junior, A. A. S. Araujo, Effects of the solid lipid nanoparticle of carvacrol on rodents with lung injury from smoke inhalation, Naunyn Schmiedebergs Arch Pharmacol 393(3) (2020) 445-455.
96. A. Mercel, N. D. Tsihlis, R. Maile, M. R. Kibbe, Emerging therapies for smoke inhalation injury: a review, J Transl Med 18(1) (2020) 141.
97. J. L. M. Dunn, L. B. Kartchner, W. H. Stepp, L. I. Glenn, M. M. Malfitano, S. W. Jones, C. M. Doerschuk, R. Maile, B. A. Cairns, Blocking CXCL1-dependent neutrophil recruitment prevents immune damage and reduces pulmonary bacterial infection after inhalation injury, Am J Physiol Lung Cell Mol Physiol 314(5) (2018) L822-L834.
98. M. Humbert, C. Guignabert, S. Bonnet, P. Dorfmüller, J. R. Klinger, M. R. Nicolls, A. J. Olschewski, S. S. Pullamsetti, R. T. Schermuly, K. R. Stenmark, M. Rabinovitch, Eur. Resp. 1 2019, 53 (1), 1801887. DOI 10.1183/13993003.01887-2018.
99. H. W. Farber, D. P. Miller, A. D. Poms, D. B. Badesch, A. E. Frost, E. M.-L. Rouzic, A. J. Romero, W. W. Benton, C. G. Elliott, M. D. McGoon, R. L. Benza, Chest 2015, 148 (4), 1043-1054. DOI 10.1378/chest.15-0300.
100. K. K. Mubarak, Respir. Med. 2010, 104 (1), 9-21. DOI 10.1016/j.rmed.2009.07.015.
101. V. Segura-Ibarra, S. Wu, N. Hassan, J. A. Moran-Guerrero, M. Ferrari, A. Guha, H. Karmouty-Quintana, E. Blanco, Front Physiol. 2018, 9, 890. DOI 10.3389/fphys.2018.00890.
102. P. J. Leary, S. Kang, T. M. Kolb, B. A. Maron, D. D. Ralph, Y. Rao, R. J. Tedford, R. T. Zamanian, Int. J. Cardiol. 2017, 240, 386-391. DOI https://doi.org/10.1016/j.ijcard.2017.04.016.
103. J. D. Duarte, R. L. Hanson, R. F. Machado, Future Cardiol.l 2013, 9 (3), 335-49. DOI 10.2217/fca.13.6.
104. A. Macchia, R. Marchioli, R. Marfisi, M. Scarano, G. Levantesi, L. Tavazzi, G. Tognoni, Am. Heart J. 2007, 153 (6), 1037-47. DOI 10.1016/j.ahj.2007.02.037.
105. E. Blanco, H. Shen, M. Ferrari, Nat. Biotechnol. 2015, 33 (9), 941-51. DOI 10.1038/nbt.3330.
106. V. Gupta, N. Gupta, I. H. Shaik, R. Mehvar, I. F. McMurtry, M. Oka, E. Nozik-Grayck, M. Komatsu, F. Ahsan, J. Controlled Release 2013, 167 (2), 189-199. DOI https://doi.org/10.1016/j.jconrel.2013.01.011.
107. J. M. McLendon, S. R. Joshi, J. Sparks, M. Matar, J. G. Fewell, K. Abe, M. Oka, I. F. McMurtry, W. T. Gerthoffer, J. Controlled Release 2015, 210, 67-75. DOI https://doi.org/10.1016/j.jconrel.2015.05.261.
108. B. Li, W. He, L. Ye, Y. Zhu, Y. Tian, L. Chen, J. Yang, M. Miao, Y. Shi, H. S. Azevedo, Z. Ma, K. Hao, Hypertension 2019, 73 (3), 703-711. DOI doi:10.1161/HYPERTENSIONAHA.118.11932.

109. K. Ichimura, T. Matoba, J.-i. Koga, K. Nakano, D. Funamoto, H. Tsutsui, K. Egashira, Int. Heart J. 2018, 59 (6), 1432-1444. DOI 10.1536/ihj.17-683.
110. V. Segura-Ibarra, J. Amione-Guerra, A. S. Cruz-Solbes, F. E. Cara, D. A. Iruegas-Nunez, S. Wu, K. A. Youker, A. Bhimaraj, G. Torre-Amione, M. Ferrari, H. Karmouty-Quintana, A. Guha, E. Blanco, Int. J. Pharm. 2017, 524 (1), 257-267. DOI https://doi.org/10.1016/j.ijpharm.2017.03.069.
111. T. Ishihara, E. Hayashi, S. Yamamoto, C. Kobayashi, Y. Tamura, R. Sawazaki, F. Tamura, K. Tahara, T. Kasahara, T. Ishihara, M. Takenaga, K. Fukuda, T. Mizushima, J. Controlled Release 2015, 197, 97-104. DOI https://doi.org/10.1016/j.jconrel.2014.10.029.
112. H. S. Choi, W. Liu, P. Misra, E. Tanaka, J. P. Zimmer, B. Itty Ipe, M. G. Bawendi, J. V. Frangioni, Nat. Biotechnol. 2007, 25 (10), 1165-70. DOI 10.1038/nbt1340.
113. J. D. Hartgerink, E. Beniash, S. I. Stupp, Science 2001, 294 (5547), 1684-8. DOI 10.1126/science.1063187.
114. C. E. Morgan, A. W. Dombrowski, C. M. Rubert Perez, E. S. Bahnson, N. D. Tsihlis, W. Jiang, Q. Jiang, J. M. Vercammen, V. S. Prakash, T. A. Pritts, S. I. Stupp, M. R. Kibbe, ACS Nano 2016, 10 (1), 899-909. DOI 10.1021/acsnano.5b06025.
115. E. S. Bahnson, H. A. Kassam, T. J. Moyer, W. Jiang, C. E. Morgan, J. M. Vercammen, Q. Jiang, M. E. Flynn, S. I. Stupp, M. R. Kibbe, Antioxid. Redox Signal 2016, 24 (8), 401-18. DOI 10.1089/ars.2015.6363.
116. T. J. Moyer, H. A. Kassam, E. S. Bahnson, C. E. Morgan, F. Tantakitti, T. L. Chew, M. R. Kibbe, S. I. Stupp, Small 2015, 11 (23), 2750-5. DOI 10.1002/sm11.201403429.
117. M. K. Klein, H. A. Kassam, R. H. Lee, W. Bergmeier, E. B. Peters, D. C. Gillis, B. R. Dandurand, J. R. Rouan, M. R. Karver, M. D. Struble, T. D. Clemons, L. C. Palmer, B. Gavin, T. A. Pritts, N. D. Tsihlis, S. I. Stupp, M. R. Kibbe, ACS Nano 2020. DOI 10.1021/acsnano.9b09243.
118. C. Orte, J. M. Polak, S. G. Haworth, M. H. Yacoub, N. W. Morrell, J. Pathol. 2000, 192 (3), 379-384. DOI 10.1002/1096-9896(2000)9999:9999<::AID-PATH715>3.0.CO;2-Q.
119. N. W. Morrell, E. N. Atochina, K. G. Morris, S. M. Danilov, K. R. Stenmark, J. Clin. Invest. 1995, 96 (4), 1823-33. DOI 10.1172/jci118228.
120. E. A. Oczypok, T. N. Perkins, T. D. Oury, Paediatric Respir. Rev. 2017, 23, 40-49. DOI 10.1016/j.prrv.2017.03.012.
121. D. Jia, Y. He, Q. Zhu, H. Liu, C. Zuo, G. Chen, Y. Yu, A. Lu, Cardiovasc. Res. 2017, 113 (6), 586-597. DOI 10.1093/cvr/cvx051.
122. M. Guo, X. Chen, Y. Wu, L. Zhang, W. Huang, Y. Yuan, M. Fang, J. Xie, D. Wei, Process Biochem. 2017, 63, 84-95. DOI https://doi.org/10.1016/j.procbio.2017.08.009.
123. Y. Liu, L. Zhang, M. Guo, H. Wu, J. Xie, D. Wei, Bioresources and Bioprocessing 2014, 1 (1), 17. DOI 10.1186/s40643-014-0017-5.
124. H. Ikram, A. H. Maslowski, M. G. Nicholls, E. A. Espiner, F. T. Hull, Br. Heart 1982, 48 (6), 541. DOI 10.1136/hrt.48.6.541.
125. M. U. Braun, P. Szalai, R. H. Strasser, M. M. Borst, Cardiovasc. Res. 2003, 59 (3), 658-67. DOI 10.1016/s0008-6363(03)00470-x.
126. K. Okada, M. L. Bernstein, W. Zhang, D. P. Schuster, M. D. Botney, Am. J. Respir. Crit. Care Med. 1998, 158 (3), 939-50. DOI 10.1164/ajrccm.158.3.9710007.
127. S. Rich, J. Martinez, W. Lam, K. M. Rosen, Br. Heart J. 1982, 48 (3), 272-277. DOI 10.1136/hrt.48.3.272.
128. C. V. Leier, D. Bambach, S. Nelson, J. B. Hermiller, P. Huss, R. D. Magorien, D. V. Unverferth, Circulation 1983, 67 (1), 155-161. DOI 10.1161/01.CIR.67.1.155.
129. H. Ni, L. Li, G. Liu, S.-Q. Hu, PLoS One 2012, 7 (5), e37077-e37077. DOI 10.1371/journal.pone.0037077.
130. N. He, L. Lin, G. Sha-Sha, L. Hai-Hang, J. Rui, H. Song-Qing, Curr. Anal. Chem. 2012, 8 (1), 180-185. DOI https://dx.doi.org/10.2174/15734111279842224.
131. G. Fritz, Trends Biochem. Sci. 2011, 36 (12), 625-32. DOI 10.1016/j.tibs.2011.08.008.
132. J. Xue, M. Manigrasso, M. Scalabrin, V. Rai, S. Reverdatto, D. S. Burz, D. Fabris, A. M. Schmidt, A. Shekhtman, Structure 2016, 24 (9), 1509-1522. DOI 10.1016/j.str.2016.06.021.
133. M. Koch, S. Chitayat, B. M. Dattilo, A. Schiefner, J. Diez, W. J. Chazin, G. Fritz, Structure 2010, 18 (10), 1342-52. DOI 10.1016/j.str.2010.05.017.
134. S. Banerjee, A. Friggeri, G. Liu, E. Abraham, J. Leukoc. Biol. 2010, 88 (5), 973-979. DOI 10.1189/jlb.0510262.
135. H. J. Huttunen, C. Fages, J. Kuja-Panula, A. J. Ridley, H. Rauvala, Cancer Res. 2002, 62 (16), 4805.
136. S. Lee, C. Piao, G. Kim, J. Y. Kim, E. Choi, M. Lee, Eur. J. Pharm. Sci. 2018, 114, 275-284. DOI 10.1016/j.ejps.2017.12.019.
137. R. J. Deane, Future Med. Chem. 2012, 4 (7), 915-925. DOI 10.4155/fmc.12.51.
138. E. Gospodarska, A. Kupniewska-Kozak, G. Goch, M. Dadlez, Biochim. Biophys. Acta 2011, 1814 (5), 592-609. DOI 10.1016/j.bbapap.2011.02.011.
139. N. A. Mansukhani, E. B. Peters, M. M. So, M. S. Albaghdadi, Z. Wang, M. R. Karver, T. D. Clemons, J. P. Laux, N. D. Tsihlis, S. I. Stupp, M. R. Kibbe, Macromol. Biosci. 2019, 19 (6), e1900066. DOI 10.1002/mabi.201900066.
140. E. B. Peters, N. D. Tsihlis, M. R. Karver, S. M. Chin, B. Musetti, B. T. Ledford, E. M. Bahnson, S. I. Stupp, M. R. Kibbe, Adv. Healthc. Mater. 2019, 8 (3), e1801545. DOI 10.1002/adhm.201801545.
141. M. M. So, N. A. Mansukhani, E. B. Peters, M. S. Albaghdadi, Z. Wang, C. M. R. Perez, M. R. Kibbe, S. I. Stupp, Adv. Biosyst. 2018, 2 (3). DOI 10.1002/adbi.201700123.
142. R. R. Vanderpool, A. R. Kim, R. Molthen, N. C. Chesler, J. Appl. Physiol. (1985) 2011, 110 (1), 188-98. DOI 10.1152/japplphysiol.00533.2010.
143. L. Yu, C. A. Hales, J. Vasc. Res. 2011, 48 (6), 465-75. DOI 10.1159/000327005.
144. J. Brett, A. M. Schmidt, S. D. Yan, Y. S. Zou, E. Weidman, D. Pinsky, R. Nowygrod, M. Neeper, C. Przysiecki, A. Shaw, et al., Am. J. Pathol. 1993, 143 (6), 1699-712.
145. N. Demling, C. Ehrhardt, M. Kasper, M. Laue, L. Knels, E. P. Rieber, Cell Tissue Res. 2006, 323 (3), 475-88. DOI 10.1007/s00441-005-0069-0.
146. S. Behzadi, V. Serpooshan, W. Tao, M. A. Hamaly, M. Y. Alkawareek, E. C. Dreaden, D. Brown, A. M. Alkilany, O. C. Farokhzad, M. Mahmoudi, Chem. Soc. Rev. 2017, 46 (14), 4218-4244. DOI 10.1039/c6cs00636a.
147. M. O. Chaney, W. B. Stine, T. A. Kokjohn, Y.-M. Kuo, C. Esh, A. Rahman, D. C. Luehrs, A. M. Schmidt, D. Stern, S. D. Yan, A. E. Roher, Biochimica et Biophysica Acta (BBA)—Mol. Basis Disease 2005, 1741 (1), 199-205. DOI https://doi.org/10.1016/j.bbadis.2005.03.014.
148. J. Xue, V. Rai, D. Singer, S. Chabierski, J. Xie, S. Reverdatto, D. S. Burz, A. M. Schmidt, R. Hoffmann, A. Shekhtman, Structure 2011, 19 (5), 722-32. DOI 10.1016/j.str.2011.02.013.
149. E. Frohlich, Int. J. Nanomedicine 2012, 7, 5577-91. DOI 10.2147/IJN.S36111.
150. C. J. Newcomb, S. Sur, J. H. Ortony, O. S. Lee, J. B. Matson, J. Boekhoven, J. M. Yu, G. C. Schatz, S. I. Stupp, Nat. Commun. 2014, 5, 3321. DOI 10.1038/ncomms4321.
151. J. W. Steed, P. A. Gale, Supramol. Chem.: Mol. Nanomater. Wiley: Hoboken, N.J., 2012.
152. V. I. Dodero, Z. B. Quirolo, M. A. Sequeira, Front Biosci. (Landmark Ed) 2011, 16, 61-73. DOI 10.2741/3676.
153. B. Moser, A. Megerle, C. Bekos, S. Janik, T. Szerafin, P. Birner, A.-I. Schiefer, M. Mildner, I. Lang, N. Skoro-Sajer, R. Sadushi-Kolici, S. Taghavi, W. Klepetko, H. J. Ankersmit, PLoS One 2014, 9 (9), e106440-e106440. DOI 10.1371/journal.pone.0106440.
154. J. Meloche, A. Courchesne, M. Barrier, S. Carter, M. Bisserier, R. Paulin, J. F. Lauzon-Joset, S. Breuils-Bonnet, E. Tremblay, S. Biardel, C. Racine, C. Courture, P. Bonnet, S. M. Majka, Y. Deshaies, F. Picard, S. Provencher, S. Bonnet, J. Am. Heart Assoc. 2013, 2 (1), e005157. DOI 10.1161/JAHA.112.005157.
155. V. V. McLaughlin, M. D. McGoon, Circulation 2006, 114 (13), 1417-31. DOI 10.1161/circulationaha.104.503540.
156. R. Stenmark Kurt, A. Fagan Karen, G. Frid Maria, Circulation Res. 2006, 99 (7), 675-691. DOI 10.1161/01.RES.0000243584.45145.3f.
157. D. G. S. Farmer, S. Kennedy, Pharmacol. Ther. 2009, 124 (2), 185-194. DOI https://doi.org/10.1016/j.pharmthera.2009.06.013.
158. M. A. Hofmann, S. Drury, B. I. Hudson, M. R. Gleason, W. Qu, Y. Lu, E. Lalla, S. Chitnis, J. Monteiro, M. H. Stickland, L. G. Bucciarelli, B. Moser, G. Moxley, S. Itescu, P. J. Grant, P. K. Gregersen, D. M. Stern, A. M. Schmidt, Genes Immun. 2002, 3 (3), 123-35. DOI 10.1038/sj.gene.6363861.
159. B. Moser, M. J. Szabolcs, H. J. Ankersmit, Y. Lu, W. Qu, A. Weinberg, K. C. Herold, A. M. Schmidt, Am. J. Transplant. 2007, 7 (2), 293-302. DOI 10.1111/j.1600-6143.2006.01617.x.
160. S. Bongarzone, V. Savickas, F. Luzi, A. D. Gee, J. Med. Chem. 2017, 60 (17), 7213-7232. DOI 10.1021/acs.jmedchem.7b00058.
161. K. Nakamura, M. Sakaguchi, H. Matsubara, S. Akagi, T. Sarashina, K. Ejiri, K. Akazawa, M. Kondo, K. Nakagawa, M. Yoshida, T. Miyoshi, T. Ogo, T. Oto, S. Toyooka, Y. Higashimoto, K. Fukami, H. Ito, PLoS One 2018, 13 (9), e0203046. DOI 10.1371/journal.pone.0203046.
162. M. Hughes, H. Xu, P. W. J. M. Frederix, A. M. Smith, N. T. Hunt, T. Tuttle, I. A. Kinloch, R. V. Ulijn, Soft Matter 2011, 7 (21), 10032-10038. DOI 10.1039/C1SM05981E.
163. H. S. Jang, J. H. Lee, Y. S. Park, Y. O. Kim, J. Park, T. Y. Yang, K. Jin, J. Lee, S. Park, J. M. You, K. W. Jeong, A. Shin, I. S. Oh, M. K. Kwon, Y. I. Kim, H. H. Cho, H. N. Han, Y. Kim, Y. H. Chang, S. R. Paik, K. T. Nam, Y. S. Lee, Nat. Commun. 2014, 5, 3665. DOI 10.1038/ncomms4665.
164. J. Lee, I. R. Choe, N. K. Kim, W. J. Kim, H. S. Jang, Y. S. Lee, K. T. Nam, ACS Nano 2016, 10 (9), 8263-70. DOI 10.1021/acsnano.6b00646.
165. K. S. Moon, E. Lee, Y. B. Lim, M. Lee, Chem. Commun. (Camb) 2008, (34), 4001-3. DOI 10.1039/b806559d.
166. S. Azarmi, W. H. Roa, R. Lobenberg, Adv. Drug Deliv. Rev. 2008, 60 (8), 863-75. DOI 10.1016/j.addr.2007.11.006.
167. D. J. Toft, T. J. Moyer, S. M. Standley, Y. Ruff, A. Ugolkov, S. I. Stupp, V. L. Cryns, ACS Nano 2012, 6 (9), 7956-65. DOI 10.1021/nn302503s.
168. J. Ilaysky, P. Jemian, J. Appl. Crystallogr.—J APPL CRYST 2009, 42, 347-353. DOI 10.1107/S0021889809002222.
169. M. Shah, M. R. Phillips, M. Quintana, G. Stupp, S. E. McLean, J. Sur. Res. 2018, 221, 35-42. DOI 10.1016/j.jss.2017.06.080.
170. A. E. Abbas, F. D. Fortuin, N. B. Schiller, C. P. Appleton, C. A. Moreno, S. J. Lester, J. Am. Coll. Cardiol. 2003, 41 (6), 1021-7. DOI 10.1016/s0735-1097(02)02973-x.
171. S. O. Granstam, E. Björklund, G. Wikström, M. W. Roos, Cardiovasc. Ultrasound 2013, 11, 7. DOI 10.1186/1476-7120-11-7.

1. A peptide amphiphile comprising: (a) a hydrophobic non-peptidic segment; (b) a β-sheet-forming peptide segment; (c) a charged peptide segment; (d) a targeting moiety, wherein the targeting moiety localizes to pulmonary tissue; wherein the hydrophobic non-peptidic segment is covalently attached to the N-terminus or C-terminus of the β-sheet-forming peptide segment; wherein the β-sheet-forming peptide segment is covalently attached to the targeting moiety; and wherein the charged peptide segment is covalently attached to the targeting moiety.

2. The peptide amphiphile of claim 1, wherein said targeting moiety comprises a peptide capable of localizing to an epitope of receptor for advanced glycation end-products (RAGE).

3. The peptide amphiphile of claim 2, wherein said peptide comprises a sequence with at least 80% identity to a sequence selected from SEQ ID NOs: 1-3 and 9.

4. The peptide amphiphile of claim 1, wherein said targeting moiety comprises a peptide capable of localizing to an epitope of angiotensin-converting enzyme (ACE).

5. The peptide amphiphile of claim 4, wherein said peptide comprises a sequence selected from SEQ ID NOs: 6-8.

6. The peptide amphiphile of claim 1, further comprising a therapeutic agent.

7. The peptide amphiphile of claim 6, wherein the therapeutic agent is attached via a covalent bond or a hydrophobic/hydrophilic interaction.

8. The peptide amphiphile of claim 6, wherein the therapeutic agent is a glutamine, a selectin or leukocyte adhesion molecule inhibitor, a CXCL-1 inhibitor, a perfluorohexane, an inducible nitric oxide synthase (iNOS) inhibitor, a neuronal NOS inhibitor, a peroxynitrite decomposition catalyst, a hydrogen sulfide (H₂S) via a hydrogen sulfide donor, a carvacrol, a peptide comprising SEQ ID NO: 18 or 19, N-acetylcysteine, ascorbic acid, a nitric oxide, a phosphodiesterase type 5 (PDE5) inhibitor, a tyrosine kinase inhibitor, a thiazolidinedione, a statin, or a modulator of LFA-1, ICAM-1, or reactive oxygen species.

9-16. (canceled)

17. The peptide amphiphile of claim 1, wherein the C-terminus of the β-sheet-forming peptide segment is covalently attached to the N-terminus of the charged peptide segment; and wherein the C-terminus of the charged peptide segment is covalently attached to the N-terminus of the targeting moiety.

18. A self-assembled nanomaterial comprising:
a plurality of peptide amphiphiles of claim 1,
wherein the targeting moiety localizes to receptor for advanced glycation end products (RAGE) or angiotensin-converting enzyme (ACE); and
wherein the hydrophobic non-peptidic segment is covalently attached to the N-terminus of the β-sheet-forming peptide segment.

19-22. (canceled)

23. The self-assembled nanomaterial of claim 18, further comprising a therapeutic agent.

24. The self-assembled nanomaterial of claim 23, wherein the therapeutic agent is encapsulated in a hydrophobic core of the self-assembled nanofiber.

25. The self-assembled nanomaterial of claim 23, wherein the therapeutic agent is a glutamine, a selectin or leukocyte adhesion molecule inhibitor, a CXCL-1 inhibitor, a perfluorohexane, an inducible nitric oxide synthase (iNOS) inhibitor, a neuronal NOS inhibitor, a peroxynitrite decomposition catalyst, a hydrogen sulfide (H₂S) via a hydrogen sulfide donor, a carvacrol, a peptide comprising SEQ ID NO: 18 or 19, N-acetylcysteine, ascorbic acid, nitric oxide, a phosphodiesterase type 5 inhibitor, a tyrosine kinase inhibitor, a thiazolidinedione, a statin, or a modulator of LFA-1, ICAM-1, or reactive oxygen species.

26-33. (canceled)

34. The self-assembled nanomaterial of claim 18, wherein the nanomaterial is a nanofiber.

35. (canceled)

36. The self-assembled nanomaterial of claim 18, wherein the C-terminus of the β-sheet-forming peptide segment is covalently attached to the N-terminus of the charged peptide segment; and wherein the C-terminus of the charged peptide segment is covalently attached to the N-terminus of the targeting moiety.

37. A method of treating a pulmonary injury or condition in a subject comprising, administering to the subject a composition comprising:
at least one peptide amphiphile of claim 1,
wherein the targeting moiety localizes to receptor for advanced glycation end products (RAGE) or angiotensin-converting enzyme (ACE); and
wherein the hydrophobic non-peptidic segment is covalently attached to the N-terminus of the β-sheet-forming peptide segment.

38. (canceled)

39-42. (canceled)

43. A method of treating a pulmonary injury or condition in a subject comprising, administering to the subject a composition comprising a self-assembled nanomaterial of claim 18.

44-48. (canceled)

49. A method of delivering a therapeutic agent to pulmonary tissue in a subject comprising, administering to the subject a composition comprising a self-assembled nanomaterial of claim 18.

50. A method of making a peptide amphiphile (PA) based nanomaterial which targets receptor for advanced glycation end products (RAGE) or angiotensin-converting enzyme (ACE) comprising:
synthesizing targeting PA molecules via solid phase peptide synthesis comprising contacting a RAGE-targeting peptide with a diluent PA backbone;
purifying the PA molecules;
dissolving targeting PA molecules and with a diluent PA in a molar ratio in a solvent;
removing the solvent; and
forming the nanomaterial via self-assembly by resuspending the mixture of PA molecules in liquid at physiological pH.

51-57. (canceled)

58. The method of claim 50, wherein the RAGE or ACE-targeting peptide is connected to the diluent PA backbone by a covalent bond in the resulting targeting PA molecule.
